SEQUENCE LISTING (64 sequences)

SEQ ID NO: 1 - protein, 375 aa, synthetic construct
MSLMVISMACVGFFLLQGAWTHEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVNGTFQADFPLGPATHGGTYTCFGSLHDSPYEWSDPSDPLLVSVTGNSSSSSSSPTEPSSKTGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 2 - DNA, 423 bp, synthetic construct
  source 1..423: mol_type = other DNA
atgggttgggtgtggaccttgctattcctgatggcagctgcccaaagtatccaagcacagatccagttggtgcagtctggacctgaggtgaagaagcctggagagacagtcaagatctcctgcaaggcttctgggtacaccttcacaaagtatggaatgaactgggtgaagcaggctccaggaaagggtttaaagtggatgggctggataaacaccaacactggagagtcaacatatgctgaagacttcaagggacggtttgccttctctttggaaacctctgccaacactgcctttttgcagatcaacaacctcaaaaatgaggacacggctgcatatttctgtgcaagatggggcccatacggtagtagcctttactatggtatggactactggggtcaaggaacctcagtcaccgtctct

SEQ ID NO: 3 - protein, 141 aa, synthetic construct
MGWVWTLLFLMAAAQSIQAQIQLVQSGPEVKKPGETVKISCKASGYTFTKYGMNWVKQAPGKGLKWMGWINTNTGESTYAEDFKGRFAFSLETSANTAFLQINNLKNEDTAAYFCARWGPYGSSLYYGMDYWGQGTSVTVS

SEQ ID NO: 4 - DNA, 315 bp, synthetic construct
  source 1..315: mol_type = other DNA
gacatccagatgacacaatcttcatcctccttttctatatctctaggagacagagtcaccattacttgcagggcaagtgaggacttatataatcgattagcctggtatcagcagaaaccaggaaatgctcctaggctcttaatatctggtgcaaccagtttggaaactggggttccttcaagattcagtggcagtggatctgggaaggatttcactctcagcattcccagtgttcagactgaagatgttggtacttactactgtcaacagtatcggtatactccgtggacgttcggaggaggcaccacgctgaat

SEQ ID NO: 5 - protein, 105 aa, synthetic construct
DIQMTQSSSSFSISLGDRVTITCRASEDLYNRLAWYQQKPGNAPRLLISGATSLETGVPSRFSGSGSGKDFTLSIPSVQTEDVGTYYCQQYRYTPWTFGGGTTLN

SEQ ID NO: 6 - DNA, 405 bp, synthetic construct
  source 1..405: mol_type = other DNA
atgggatggagctgtatcatgttctttctggtagcaacagctacaggtgtgcactcccaggtccagctgcagcagtctggggctgagctggtgaggcctggggtctcagtgaagatttcctgcaagggttctggctacacattcactgattatactatgcactgggtgaagcagagtcatgcaaagagtctagagtggattggagttattagtccttactatggtgatgctagctacaaccagaagttcaagggcaaggccacaatgactgtagacaaatcctccagcacagcctatatggaacttgccagactgacatctgaagattctgccatctattactgtgcaagagggttactacgtgggtttgcttactggggccaagggactctggtcactgtctct

SEQ ID NO: 7 - protein, 135 aa, synthetic construct
MGWSCIMFFLVATATGVHSQVQLQQSGAELVRPGVSVKISCKGSGYTFTDYTMHWVKQSHAKSLEWIGVISPYYGDASYNQKFKGKATMTVDKSSSTAYMELARLTSEDSAIYYCARGLLRGFAYWGQGTLVTVS

SEQ ID NO: 8 - DNA, 390 bp, synthetic construct
  source 1..390: mol_type = other DNA
tgggatttgagggagtcacagactcaggtcttcatgtttcttatgttcagggtattgggtgcctgtgcagacattgtgatgacacagtctccatcctccaaggatatgtcagtaggacagaaggtcactatgaggtgcaagtccagtaagagccttttaaatagtagcaatcaaaagaaatatttggcctggtaccagcagaaaccaggacagtctcctaaacttctggtatactttgcatccattagggaatctggggtccctgatcgcttcataggcagtggatctgggacagatttcactcttaccatcagcagtgtgcaggttgaagacctggcagattacttctgtcagcaacattatagcactccgtggacgttcggtggaggc

SEQ ID NO: 9 - protein, 130 aa, synthetic construct
WDLRESQTQVFMFLMFRVLGACADIVMTQSPSSKDMSVGQKVTMRCKSSKSLLNSSNQKKYLAWYQQKPGQSPKLLVYFASIRESGVPDRFIGSGSGTDFTLTISSVQVEDLADYFCQQHYSTPWTFGGG

SEQ ID NO: 10 - DNA, 414 bp, synthetic construct
  source 1..414: mol_type = other DNA
atgggatggagctgtatcatgttcttcctagtggcaacagctataggtgtccactcccaggttcagcttcagcagtctgggggtgagctggtgaagcctggggcctcagtgaagatgtcctgcaaggcttttggctacaccttcacttcctttccaatagagtggatgaagcagaatcatgggaagagcctagagtggattggaaattttcatccttataatgatgatactaagtacaatgaaaaattcaacggcaaggccaaattgactgcagaaacatcctctaaaacagtctatttggagctcagccgattaacatctgatgactgcagtgtttattactgtgcaagagggagtgattattcgtttggtttggactactggggtcaaggaacctcagtcaccgtctcctca

SEQ ID NO: 11 - protein, 138 aa, synthetic construct
MGWSCIMFFLVATAIGVHSQVQLQQSGGELVKPGASVKMSCKAFGYTFTSFPIEWMKQNHGKSLEWIGNFHPYNDDTKYNEKFNGKAKLTAETSSKTVYLELSRLTSDDCSVYYCARGSDYSFGLDYWGQGTSVTVSS

SEQ ID NO: 12 - DNA, 396 bp, synthetic construct
  source 1..396: mol_type = other DNA
gtattcagggagacagacacactcatgctatgggtggtgctgctctgggttccaggttccacaggtggcattgtgctgacccaatctccagcttctttggttgtgtctctagggcagagggccaccatatcttgcagagccagtgaaagtgttgatagttatggctatagttttatgcactggtaccagcagaaaccaggacagccacccaaactcctcatttatcgtgcatccaacctaaaatctgggatccctgccaggttcagtggcagtgggtgtaggacagacttcaccctcaccattaatcctgtggaggctgatgatgttgcaacctattactgtcagcaaagtaatgaggatccttggacgttcggtggaggcaccaaagctggaaat

SEQ ID NO: 13 - protein, 132 aa, synthetic construct
VFRETDTLMLWVVLLWVPGSTGGIVLTQSPASLVVSLGQRATISCRASESVDSYGYSFMHWYQQKPGQPPKLLIYRASNLKSGIPARFSGSGCRTDFTLTINPVEADDVATYYCQQSNEDPWTFGGGTKAGN

SEQ ID NO: 14 - DNA, 417 bp, synthetic construct
  source 1..417: mol_type = other DNA
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagcaacaatggtcgcgctaggtataatccatctctcaaaagtcgaatctctatcactcgagacacattcaagaaccagttcttcctgcagttgaattctgtgactactgaggacacagccacatattactgtgcaagagaggcctcgcatgatggttccttctggtacttcgatgtctggggcgcagggaccacggtcaccgtctct

SEQ ID NO: 15 - protein, 139 aa, synthetic construct
MRVLILLWLFTAFPGILSDVQLQESGPGLVKPSQSLSLTCTVTGYSITSDYAWNWIRQFPGNKLEWMGYISNNGRARYNPSLKSRISITRDTFKNQFFLQLNSVTTEDTATYYCAREASHDGSFWYFDVWGAGTTVTVS

SEQ ID NO: 16 - DNA, 333 bp, synthetic construct
  source 1..333: mol_type = other DNA
gacagtgtgttgacccaatctccagcttgtttggttgtgtgtatagggcagagggccaccatatcttgcagagccagtgaaagtgttgatagttatggctatagttttatgcattggtaccagcagaaaccaggacagccacccaaactcctcatttatcgtgcatccaacctagaatgtgggatccctgccaggttcagtggcagggggtctaggacggacttcaccctcaccattactcctgtggagggtgatgatgttgcaacctattactgtcagcaaagtaatgaggatcctcggacgttcggtggaggcaccaagctggaaatcaaa

SEQ ID NO: 17 - protein, 111 aa, synthetic construct
DSVLTQSPACLVVCIGQRATISCRASESVDSYGYSFMHWYQQKPGQPPKLLIYRASNLECGIPARFSGRGSRTDFTLTITPVEGDDVATYYCQQSNEDPRTFGGGTKLEIK

SEQ ID NO: 18 - DNA, 414 bp, synthetic construct
  source 1..414: mol_type = other DNA
atgggatggagctgtatcatgttcttcctagtggcaacagctataggtgtccactcccaggttcagcttcagcagtctggggctgagctggtgaagcctggggcctcagtgaagatgtcctgcaaggcttttggctacaccttcacttcctttccaatagagtggatgaagcagaatcatgggaagagcctagagtggattggaaattttcatccttctaatgatgatactaagtacaatgaaaaattcaacggcaaggccaaattgactgcagaaacatcctctaaaacagtctatttggagctcagccgattaacatctgatgactctgctgtttattactgtgcaagagggagtgattattcctttgctttggactactggggtcaaggaacctcagtcaccgtctcctca

SEQ ID NO: 19 - protein, 138 aa, synthetic construct
MGWSCIMFFLVATAIGVHSQVQLQQSGAELVKPGASVKMSCKAFGYTFTSFPIEWMKQNHGKSLEWIGNFHPSNDDTKYNEKFNGKAKLTAETSSKTVYLELSRLTSDDSAVYYCARGSDYSFALDYWGQGTSVTVSS

SEQ ID NO: 20 - DNA, 393 bp, synthetic construct
  source 1..393: mol_type = other DNA
atggaactagtcgacatggttcttatgttgctgctgctatgggttccaggttccacaggtggcattgtgctgacccaatctccagcttctttggctgtgtctctagggcagagggccaccatatcctgcagagccagtgaaagtgttgatagttatggctatagttttatgcactggtaccagcagaaaccaggacagccacccaaactcctcatctatcgtgcatccaacctaaaatctgggatccctgccaggttcagtggcagtgggtctaggacagacttcaccctcaccattaatcctgtggaggctgatgatgttgcaacctattactgtcagcaaagtaatgaggatccttggacgttcggtggaggcaccaagctggaaatcaaa

SEQ ID NO: 21 - protein, 131 aa, synthetic construct
MELVDMVLMLLLLWVPGSTGGIVLTQSPASLAVSLGQRATISCRASESVDSYGYSFMHWYQQKPGQPPKLLIYRASNLKSGIPARFSGSGSRTDFTLTINPVEADDVATYYCQQSNEDPWTFGGGTKLEIK

SEQ ID NO: 22 - DNA, 426 bp, synthetic construct
  source 1..426: mol_type = other DNA
atgggttgggtgtggaccttgctattcctgatggcagctgcccaaagtatccaagcacagatccagttggtgcagtctggacctgagctgaagaagcctggagagacagtcaagatctcctgcaaggcttctggatataccttcacaaactatggaatgaactgggtgaagcaggctccaggaaagggtttaaagtggatgggctggataaacaccaacactggagagacaacatatgctgaagaggtcaagggacggtttgccttctctttggaaacctctgccagcactgcctatttgcagatcaacaacctcaaaaatgaggacacggctacatatttctgtgcaagatggggcccatacggtagtagcctttatttttctatggactactggggtcaaggaacctcagtcaccgtctcctca

SEQ ID NO: 23 - protein, 142 aa, synthetic construct
MGWVWTLLFLMAAAQSIQAQIQLVQSGPELKKPGETVKISCKASGYTFTNYGMNWVKQAPGKGLKWMGWINTNTGETTYAEEVKGRFAFSLETSASTAYLQINNLKNEDTATYFCARWGPYGSSLYFSMDYWGQGTSVTVSS

SEQ ID NO: 24 - DNA, 324 bp, synthetic construct
  source 1..324: mol_type = other DNA
gatatccagatgacacaatcttcatcctccttttctgtatctctaggagacagactcaccattacttgcaaggcaagtgaggacatatataatcggttagcctggtatcaacagaaaccaggaaatgctcctaggctcttaatatatggtgcaaccagtttggaaagtggggttccttcaagattcagtggcagtggatctggaaaggattacactctcagcattcccagttttcagagagaagatggtggtagcaacttatgtcaacagtatcggaatagagcgtggacgttcggaggagggaccaagctggaaataaaacgg

SEQ ID NO: 25 - protein, 108 aa, synthetic construct
DIQMTQSSSSFSVSLGDRLTITCKASEDIYNRLAWYQQKPGNAPRLLIYGATSLESGVPSRFSGSGSGKDYTLSIPSFQREDGGSNLCQQYRNRAWTFGGGTKLEIKR

SEQ ID NO: 26 - DNA, 405 bp, synthetic construct
  source 1..405: mol_type = other DNA
atggcttgggtgtggaccttgctattcctgatggcagctgcccaaagtatccaagcacagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgggccaacatatgccgatgacttcaagggacggtttgccttctctttggaaacctctgccaccactgcctatttgcagatcaacaacctcaaaaatgaggacacggctacatatttctgtagtagagatgtcgacctctactttgactactggggccaaggcaccactctcacagtctct

SEQ ID NO: 27 - protein, 135 aa, synthetic construct
MAWVWTLLFLMAAAQSIQAQIQLVQSGPELKKPGETVKISCKASGYTFTDYSMHWVKQAPGKGLKWMGWINTETGGPTYADDFKGRFAFSLETSATTAYLQINNLKNEDTATYFCSRDVDLYFDYWGQGTTLTVS

SEQ ID NO: 28 - DNA, 321 bp, synthetic construct
  source 1..321: mol_type = other DNA
gacatccagatgactcagtctccagcctccctatctgcatctgtgggagaaactgtcaccatcacatgtcgaacaagtgagaatatttacagttatttagcatggtatcagcagaaacagggaaaatctcctcagctcctggtctataatgcaaaaaccctggtagaaggtgtgccatcgaggttcagtggcagtggatcaggcacacagttttctgtgaagatcaacagcctgcagcctgaagattttgggaattattactgtcaacatcattatgggattccgttcacgttcggaggagggaccaaactagaaataaaa

SEQ ID NO: 29 - protein, 107 aa, synthetic construct
DIQMTQSPASLSASVGETVTITCRTSENIYSYLAWYQQKQGKSPQLLVYNAKTLVEGVPSRFSGSGSGTQFSVKINSLQPEDFGNYYCQHHYGIPFTFGGGTKLEIK

SEQ ID NO: 30 - DNA, 423 bp, synthetic construct
  source 1..423: mol_type = other DNA
atgggttgggtgtggaccttgctattcctgatggcagctgcccaaagtatccaagcacagatccagttggtgcagtctggacctgaggtgaagaagcctggagagacagtcaagatctcctgcaaggcttctgggtataccttcacaaagtatggaatgaactgggtgaagcaggctccaggaaagggtttaaagtggatgggctggataaacaccaacactggagagccaacatatgctgaagagttcaagggacggtttgccttctctttggaaacctctgccagcactgcctttttgcagatcaacaacctcaaaaatgaggacacggctgcatatttctgtgcaagatggggcccatacggtagtagcctttactatgctatggactactggggtcaaggaacctcagtcaccgtctct

SEQ ID NO: 31 - protein, 141 aa, synthetic construct
MGWVWTLLFLMAAAQSIQAQIQLVQSGPEVKKPGETVKISCKASGYTFTKYGMNWVKQAPGKGLKWMGWINTNTGEPTYAEEFKGRFAFSLETSASTAFLQINNLKNEDTAAYFCARWGPYGSSLYYAMDYWGQGTSVTVS

SEQ ID NO: 32 - DNA, 324 bp, synthetic construct
  source 1..324: mol_type = other DNA
gacatccagatgacacaatcttcatcctccttttctgtatctgtaggagacagagtcaccattacttgcagggcaagtgaggacatatataatcggttagcctggtatcagcagaaaccaggaaatgctcctaggctcttaatatctggtgcaaccagtttggaaactggggttccttcaagattcagtggcagtggatctgggaaggattacactctcagcattcccagtgttcagagagaagatggaggtagcaacttatgtcagccatcacggagtagaccgtgcacgttcggaggaggcaccaagctgaaatcaaagcga

SEQ ID NO: 33 - protein, 108 aa, synthetic construct
DIQMTQSSSSFSVSVGDRVTITCRASEDIYNRLAWYQQKPGNAPRLLISGATSLETGVPSRFSGSGSGKDYTLSIPSVQREDGGSNLCQPSRSRPCTFGGGTKLKSKR

SEQ ID NO: 34 - DNA, 28 bp, synthetic construct
  source 1..28: mol_type = other DNA
ggtctatttgggaaaccttcactctcag

SEQ ID NO: 35 - DNA, 18 bp, synthetic construct
  source 1..18: mol_type = other DNA
tgtccaggccccctgcag

SEQ ID NO: 36 - DNA, 23 bp, synthetic construct
  source 1..23: mol_type = other DNA
ggaaactcttcaagtagttcatc

SEQ ID NO: 37 - DNA, 18 bp, synthetic construct
  source 1..18: mol_type = other DNA
tgtgaccacgatcaccag

SEQ ID NO: 38 - DNA, 23 bp, synthetic construct
  source 1..23: mol_type = other DNA
gcccagcgtcgatggaacattcc

SEQ ID NO: 39 - DNA, 19 bp, synthetic construct
  source 1..19: mol_type = other DNA
actgcagggagcctaggtt

SEQ ID NO: 40 - DNA, 25 bp, synthetic construct
  source 1..25: mol_type = other DNA
cacatgcttcagctctctccatgac

SEQ ID NO: 41 - DNA, 18 bp, synthetic construct
  source 1..18: mol_type = other DNA
taggtccctccgtgggtg

SEQ ID NO: 42 - DNA, 23 bp, synthetic construct
  source 1..23: mol_type = other DNA
gctcatggtcgtcagcatggcgt

SEQ ID NO: 43 - DNA, 21 bp, synthetic construct
  source 1..21: mol_type = other DNA
gacatagatctaatccggcgc

SEQ ID NO: 44 - DNA, 23 bp, synthetic construct
  source 1..23: mol_type = other DNA
gggggcctggccacatgagggtg

SEQ ID NO: 45 - DNA, 24 bp, synthetic construct
  source 1..24: mol_type = other DNA
tgcagcaagaagaacccaacacac

SEQ ID NO: 46 - DNA, 23 bp, synthetic construct
  source 1..23: mol_type = other DNA
cctggtgatcatggtcacaggtc

SEQ ID NO: 47 - DNA, 18 bp, synthetic construct
  source 1..18: mol_type = other DNA
gggttgctgggtgctgac

SEQ ID NO: 48 - DNA, 24 bp, synthetic construct
  source 1..24: mol_type = other DNA
ggacatgtgagtcttctgtgtcgc

SEQ ID NO: 49 - DNA, 18 bp, synthetic construct
  source 1..18: mol_type = other DNA
tcctcgaggcaccacagc

SEQ ID NO: 50 - DNA, 23 bp, synthetic construct
  source 1..23: mol_type = other DNA
tgtcgctctcatcttgggtttac

SEQ ID NO: 51 - DNA, 18 bp, synthetic construct
  source 1..18: mol_type = other DNA
cagaagagtcacatgtcc

SEQ ID NO: 52 - DNA, 23 bp, synthetic construct
  source 1..23: mol_type = other DNA
cagatgtcggagttcacacccac

SEQ ID NO: 53 - DNA, 18 bp, synthetic construct
  source 1..18: mol_type = other DNA
taggtccctgcgtgtgca

SEQ ID NO: 54 - DNA, 21 bp, synthetic construct
  source 1..21: mol_type = other DNA
acccagcaactccctggtgat

SEQ ID NO: 55 - DNA, 18 bp, synthetic construct
  source 1..18: mol_type = other DNA
gctgaccactcaatgggg

SEQ ID NO: 56 - DNA, 23 bp, synthetic construct
  source 1..23: mol_type = other DNA
ggaggtgacatttgcacagttgg

SEQ ID NO: 57 - DNA, 23 bp, synthetic construct
  source 1..23: mol_type = other DNA
ggaggtgacatttgcacagttgg

SEQ ID NO: 58 - DNA, 23 bp, synthetic construct
  source 1..23: mol_type = other DNA
taccaccatgttcatggaacttc

SEQ ID NO: 59 - DNA, 18 bp, synthetic construct
  source 1..18: mol_type = other DNA
tctgttggaggtgtcttg

SEQ ID NO: 60 - DNA, 50 bp, synthetic construct
  source 1..50: mol_type = other DNA
tgtcgtgaggaattgatccttcgaactagtatgtcgctcatggtcatcag

SEQ ID NO: 61 - DNA, 50 bp, synthetic construct
  source 1..50: mol_type = other DNA
tgtaagtcattggtcttaaaggtacctgaggtcagattccagctgctggt

SEQ ID NO: 62 - DNA, 20 bp, synthetic construct
  source 1..20: mol_type = other DNA
gcactaccagagctaactca

SEQ ID NO: 63 - DNA, 20 bp, synthetic construct
  source 1..20: mol_type = other DNA
gatgttcgggttgcgcgtag

SEQ ID NO: 64 - DNA, 20 bp, synthetic construct
  source 1..20: mol_type = other DNA
gatgttcgggttgcgcgtag

(19) United States
(12) Patent Application Publication
(10) Pub. No.: US 2025/0019435 A1
(43) Pub. Date: Jan. 16, 2025

(54) METHODS, COMPOSITIONS, AND KITS FOR MODIFYING IMMUNE CELL ACTIVITY VIA KIR2DL5

(71) Applicant: Albert Einstein College of Medicine, Bronx, NY (US)

(72) Inventors: Xingxing ZANG, New York, NY (US); Xiaoxin REN, Bronx, NY (US); Yao WEI, New York, NY (US)

(21) Appl. No.: 18/708,555

(22) PCT Filed: Nov. 7, 2022
(86) PCT No.: PCT/US2022/079401

§ 371 Date: May 8, 2024

Related U.S. Application Data
(60) Provisional application No. 63/263,710, filed on Nov. 8, 2021.

(51) Int. Cl. C07K 16/28; A61K 39/00; A61P 35/00

(52) U.S. Cl. CPC C07K 16/2803; A61P 35/00; A61K 2039/505; C07K 2317/76; C07K 2317/92

(57) ABSTRACT

Provided herein are methods of modifying immune cell activity by altering KJR2DL5 expression and/or activity. The methods, which include administering to a subject one or more agents that decrease KJR2DL5 expression and/or activity, increase immune cell function, and treat diseases such as cancer and infectious diseases.

### CROSS-REFERENCE TO RELATED APPLICATION

This application claims priority to U.S. Provisional Application No. 63/263,710, filed on Nov. 8, 2021, the disclosure of which is hereby incorporated by reference herein in its entirety.

### SEQUENCE LISTING

This application contains an ST.26 compliant Sequence Listing, which was submitted in xml format via Patent Center and is hereby incorporated by reference in its entirety. The .xml copy, created on Oct. 28, 2022 is named 129807.8027.W000 Sequence Listing.xml and is 64 KB in size.

### BACKGROUND

Human killer-cell immunoglobulin-like receptors (KIRs) regulate Natural killer (NK) cells' activity by recognizing self-HLA class I molecules (Wagtmann 1995; Vilches 2002). KIR2DL5 is a member of the KIR family, but its biological functions are largely unknown (Winter 1998; Frazier 2013; Vilches 2000; Cisneros 2016; Gomez-Lozano 2002; Du 2008).

Poliovirus receptor (PVR, also known as CD155) is a member of the nectin/nectin-like family, which mediates cell adhesion, invasion and migration, and proliferation (Verschueren 2020; Husain 2019; Shilts 2022; Takai 2008; Kucan Brlic 2019). PVR overexpression induces tumor cell immune escape and is associated with a poor prognosis and enhanced tumor progression (Triki 2019; Carlsten 2007; Castriconi 2004; Masson 2001). Besides its tumor-intrinsic roles, PVR participates in multiple immunoregulatory events through interaction with the stimulatory receptor DNAX accessory molecule 1 (DNAM-1, also known as CD226) and the inhibitory receptors T cell immunoreceptor with Ig and ITIM domains (TIGIT) and CD96 (Bottino 2003; Yu 2009; Chan 2014). Certain immunotherapies targeting the TIGIT/PVR axis as a potential cancer therapy are in clinical trials (Bendell 2020; Niu 2022; Cohen 2021; Wainberg 2021; Rodriguez-Abreu 2020; Ge 2021). Alternative approaches targeting other PVR pathways could contribute to improved outcomes. Accordingly, the present technology provides therapeutic strategies targeting the KIR2DL5/PVR pathway in the tumor microenvironment (TME) to satisfy an urgent need in the field.

### SUMMARY

Provided herein in certain embodiments are methods of modifying immune cell activity by altering KIR2DL5 expression and/or activity.

In one aspect, the present disclosure provides a method of increasing immune cell function in a subject comprising administering to the subject one or more agents that decrease KIR2DL5 expression and/or activity.

In another aspect, the present disclosure provides a method of treating an infectious disease in a subject in need thereof comprising administering to the subject one or more agents that decrease KIR2DL5 expression and/or activity.

In yet another aspect, the present disclosure provides a method of treating cancer in a subject in need thereof comprising administering one or more agents that decrease KIR2DL5 expression and/or activity.

In some embodiments, the one or more agents prevent or reduce KIR2DL5 binding to poliovirus receptor (PVR). In certain of these embodiments, the one or more agents binds KIR2DL5 at or near its binding site for PVR. In certain of these embodiments, the one or more agents bind PVR at or near its binding site for KIR2DL5.

In some embodiments, binding of the one or more agents to PVR does not block PVR binding to TIGIT, DNAM-1, and CD96.

In some embodiments, the one or more agents is selected from a peptide, polypeptide, or small molecule. In certain of these embodiments, the polypeptide is an antibody or a fusion protein comprising said antibody. In some embodiments, the antibody is a monoclonal antibody.

In yet another embodiment, the antibody is an antagonist antibody. In some embodiments, the antibody is a chimeric antibody, a human antibody, or a humanized antibody.

In some embodiments, the antibody or fusion protein comprising said antibody comprises a high chain variable region (VH) comprising an amino acid sequence encoded by SEQ ID NO:2, SEQ ID NO:6, SEQ ID NO:10, SEQ ID NO:14, SEQ ID NO:18, SEQ ID NO:22, SEQ ID NO:26, or SEQ ID NO:30.

In some embodiments, the antibody or fusion protein comprising said antibody comprises a VH region comprising an amino acid sequence encoded by a nucleotide sequence that is at least 80% identical to SEQ ID NO:2, SEQ ID NO:6, SEQ ID NO:10, SEQ ID NO:14, SEQ ID NO:18, SEQ ID NO:22, SEQ ID NO:26, or SEQ ID NO:30.

In some embodiments, the antibody or fusion protein comprising said antibody comprises a VH region comprising an amino acid of SEQ ID NO:3, SEQ ID NO:7, SEQ ID NO:11, SEQ ID NO:15, SEQ ID NO:19, SEQ ID NO:23, SEQ ID NO:27, or SEQ ID NO:31.

In some embodiments, the antibody or fusion protein comprising said antibody comprises a VH region comprising an amino acid sequence that is at least 80% identical to SEQ ID NO:3, SEQ ID NO:7, SEQ ID NO:11, SEQ ID NO:15, SEQ ID NO:19, SEQ ID NO:23, SEQ ID NO:27, or SEQ ID NO:31.

In another embodiment, the antibody or fusion protein comprising said antibody comprises a light chain variable region (LH) comprising an amino acid sequence encoded by SEQ ID NO:4, SEQ ID NO:8, SEQ ID NO:12, SEQ ID NO:16, SEQ ID NO:20, SEQ ID NO:24, SEQ ID NO:28, or SEQ ID NO:32.

In some embodiments, the antibody or fusion protein comprising said antibody comprises a LH region comprising an amino acid sequence encoded by a nucleotide sequence that is at least 80% identical to SEQ ID NO:4, SEQ ID NO:8, SEQ ID NO:12, SEQ ID NO:16, SEQ ID NO:20, SEQ ID NO:24, SEQ ID NO:28, or SEQ ID NO:32.

In some embodiments, the antibody or fusion protein comprising said antibody comprises a LH region comprising an amino acid of SEQ ID NO:5, SEQ ID NO:9, SEQ ID NO:13, SEQ ID NO:17, SEQ ID NO:21, SEQ ID NO:25, SEQ ID NO:29, or SEQ ID NO:33.

In some embodiments, the antibody or fusion protein comprising said antibody comprises a LH region comprising an amino acid sequence that is at least 80% identical to SEQ ID NO:5, SEQ ID NO:9, SEQ ID NO:13, SEQ ID NO:17, SEQ ID NO:21, SEQ ID NO:25, SEQ ID NO:29, or SEQ ID NO:33.

In some embodiments, the infectious disease is caused by a pathogen. In certain of these embodiments, the pathogen is selected from a virus, bacterium, prion, fungus, parasite, or combination thereof. In some embodiments, the virus is selected the group consisting of human immunodeficiency viruses, influenza viruses, papillomaviruses, coronaviruses, hepatitis viruses, and herpesviruses. In some embodiments, the bacterium is Mycobacterium tuberculosis. In some embodiments, the fungus is Pneumocystis jirovecii (PJP).

In some embodiments, the cancer is selected from the group consisting of chronic lymphocytic leukemia (CLL), acute leukemia, acute lymphoid leukemia (ALL), B-cell acute lymphoid leukemia (B-ALL), T-cell lymphoma, B-cell lymphoma, T-cell acute lymphoid leukemia (T-ALL), chronic myelogenous leukemia (CML), B-cell prolymphocytic leukemia, T-cell lymphoma, Hodgkin's disease, B-cell non-Hodgkin's lymphoma, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, mucosa-associated lymphoid tissue (MALT) lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

In yet another embodiment, the cancer is selected from the group consisting of colon cancer, rectal cancer, renal-cell carcinoma, liver cancer, lung cancer, kidney cancer, gastric cancer, gallbladder cancer, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers, combinations of the cancers, and metastatic lesions of the cancers.

In some embodiments, the cancer is a human hematologic malignancy. In certain of these embodiments, the human hematologic malignancy is selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt's lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some embodiments, the cancer is selected from the group consisting of bladder cancer, kidney cancer, breast cancer, lung cancer, liver cancer, brain cancer, prostate cancer, colon cancer, esophageal cancer, pancreatic cancer, uterine cancer, and stomach cancer.

In some embodiments, the cancer is a metastatic cancer.

In some embodiments, the method further comprises administering the subject to one or more additional cancer therapies selected from chemotherapy, radiation therapy, immunotherapy, surgery, and a combination thereof.

In another aspect, the present disclosure provides a method of decreasing immune cell function in a subject comprising administering to the subject one or more agents that increase KIR2DL5 expression and/or activity.

In some aspects, the present disclosure provides a method of treating an autoimmune disease in a subject comprising administering to the subject one or more agents that increase KIR2DL5 expression and/or activity to the subject.

In yet another aspect, the present disclosure provides a method of decreasing transplant rejection in a subject comprising administering to the subject one or more agents that increase KIR2DL5 expression and/or activity to the subject.

In some embodiments, the one or more agents is selected from the group consisting of a peptide, polypeptide, and a small molecule. In certain of these embodiments, the polypeptide is a fusion protein or an antibody. In some embodiments, the antibody is a monoclonal antibody.

In some embodiments, the antibody is an agonist antibody. In some embodiments, the antibody increases activity of KIR2DL5. In some embodiments, the antibody is a chimeric antibody, a human antibody, or a humanized antibody.

In some embodiments, the autoimmune disease is selected from the group consisting of acute disseminated encephalomyelitis (ADEM), alopecia areata, antiphospholipid syndrome, autoimmune cardiomyopathy, autoimmune hemolytic anemia, autoimmune hepatitis, autoimmune inner ear disease, autoimmune lipoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Behçet's disease, celiac disease, Chagas disease, cold agglutinin disease, Crohn's disease, dermatomyositis, diabetes mellitus type 1, eosinophilic fasciitis, gastrointestinal pemphigoid, Goodpasture's syndrome, Grave's syndrome, Guillain-Barré syndrome, Hashimoto's encephalopathy, Hashimoto's thyroiditis, lupus erythematosus, Miller-Fisher syndrome, mixed connective tissue disease, myasthenia gravis, pemphigus vulgaris, pernicious anemia, polymyositis, psoriasis, psoriatic arthritis, relapsing polychondritis, rheumatoid arthritis, rheumatic fever, Sjögren's syndrome, temporal arteritis, transverse myelitis, ulcerative colitis, undifferentiated connective tissue disease, vasculitis, Wegener's granulomatosis, and adult rheumatoid arthritis.

In some embodiments, the transplant is a stem cell transplant, bone marrow transplant, or combination thereof. In certain of these embodiments, the transplant is selected from the group consisting of a kidney transplant, a lung transplant, a heart transplant, a pancreas transplant, a cornea transplant, or a liver transplant.

### BRIEF DESCRIPTION OF THE DRAWINGS

FIG. 1: Flow cytometric analysis of PVR binding to KIR2DL5/3T3 or KIR2DL4/3T3 at increasing concentrations of PVR-Ig.

FIG. 2: Flow cytometric analysis of PVR binding on 3T3 cells expressing wild-type KIR2DL5 and indicated variants.

FIG. 3: PVR/3T3 cells were preincubated with CD226-His, CD96-His, TIGIT-His and TMIGD2-His (negative control) tag protein at the indicated concentrations and then stained by KIR2DL5-human Ig fusion protein.

FIG. 4A-4C: A. FACS analysis of PBMC shows that KIR2DL5 is expressed on the cell surface of human innate immune cells (NK cells, γδ T cells) and adaptive immune cells (CD8+ T cells, CD4+ T cells). B. Percentage of KIR2DL5 positive cells on the indicated NK cell subsets from eight different donors. C. Percentage of KIR2DL5+ CD8 T cells on the indicated CD8 T cell subsets from eight different donors.

FIG. 5A-5D. KIR2DL5 inhibits NK cell function and mediates PVR⁺ tumor immune resistance. (A-C) Redirected cytotoxicity of primary KIR2DL5⁺ NK cells against P815. A. The lysis of P815 cells (n=4). B. Degranulation (CD107a) and cytokine production (IFN-γ and TNF-α) of primary KIR2DL5⁺ NK cells (n=4). CD56 served as negative control. C. Cytokine and chemokine production in the co-culture supernatant of primary KIR2DL5⁺ NK cells with the indicated antibody-coated P815 (n=5). D. Effects of anti-KIR2DL5 blocking mAb F8B30 or control mIgG1 on the lysis of scrambled control or PVR^{KO} A427 or Jurkat cells by primary KIR2DL5+ NK cells at indicated E:T ratios.

FIG. 6A-6F: A-B. A427 subcutaneous tumor model. NSG mice were engrafted s.c. with A427 on day 0, followed by randomization on day 3 and i.t. treatment with KIR2DL5+ NK cells together with mIgG1 or F8B30 every three days for twice. Growth of A427 tumors (A) and Kaplan-Meier survival curves of mice (B). n=8 tumors per group. C-D. A427 xenograft lung tumor model. NSG mice were engrafted i.v. with A427 on day 0, followed by randomization on day 1 and i.v. treatment with KIR2DL5+ NK cells together with mIgG1 or F8B30 every three days for twice. Tumor growth in the lung monitored by means of bioluminescence imaging (C) and Kaplan-Meier survival curves of mice (D). n=5 mice per group. E-F. Jurkat xenograft tumor model. NSG mice were engrafted i.v. with Jurkat on day 0, followed by randomization on day 4 and i.v. treatment with KIR2DL5+ NK cells together with mIgG1 or F8B30 every three days for twice. Tumor growth in vivo monitored by means of bioluminescence imaging (E) and Kaplan-Meier survival curves of mice (F). n=6 mice per group.

FIG. 7A-7B: A. Expression and phosphorylation of Vav1, ERK1/2, p90RSK, and NF-κB in primary KIR2DL5+ NK cells after crosslinking with indicated mAbs at the indicated timepoints. B. Quantification of immunoblotting.

FIG. 8A-8D: Generation and characterization of anti-KIR2DL5-specific mAbs. (A) The specificity of anti-KIR2DL5 mAb clone F8B30. 3T3 cells transduced with indicated KIR family members were stained with 5 μg/mL of F8B30 (open) or mIgG1 (shaded). (B) 3T3 cells transduced with DO-deleted (KIR2DL5 dD0) or D2-deleted KIR2DL5 (KIR2DL5 dD2) were stained with 5 μg/mL of clone F8B30 or commercial clone UP-R1. (C) Anti-KIR2DL5 mAb clone F8B30 recognized different KIR2DL5A and 5B alleles. 3T3 cells transduced with indicated alleles were stained with 5 μg/mL of F8B30 or UP-R1 (open) or mIgG1 (shaded). (D) Anti-KIR2DL5 mAb clone F8B30 recognized different KIR2DL5 DO domain variants. 3T3 cells transduced with indicated DO variants were stained with 0.025 μg/mL of F8B30 or UP-R1 (open) or mIgG1 (shaded). In A-D, data are representative of 2 independent experiments.

FIG. 9A-9F: KIR2DL5 was expressed on human innate and adaptive immune cells. (A) KIR2DL5 expression on human PBMCs. The frequencies of KIR2DL5⁺ cells in the indicated subsets (n=17 for NK and CD8⁺ T; n=15 for CD4⁺ T and γδ T). Data are represented as mean±SEM. FIG. 4A (see above) shows Flow cytometric analysis of KIR2DL5 expression on the indicated subsets from 1 donor. (B) The distribution of KIR2DL5⁺ CD8⁺ T cells on the indicated cell subsets based on CD45RA and CCR7 expression. FIG. 4C (see above) is a summary of KIR2DL5⁺ CD8⁺ T cell distribution (n=8). Data are represented as mean±SEM. (C) KIR2DL5 expression on CD56^{bright}CD16⁻ and CD56^{dim}CD16⁺ NK subsets. The frequencies of KIR2DL5⁺ cells on the indicated NK cell subsets are shown on FIG. 4B (n=8) (see above). (D) KIR2DL5 expression on CD56^{dim}CD57⁻ and CD56^{dim}CD57⁺ NK subsets. The frequencies of KIR2DL5⁺ cells on the indicated NK cell subsets (n=8) are shown on the right. (E) Flow cytometric analysis of coexpression pattern of KIR2DL5 with DNAM-1, TIGIT, and CD96 on primary resting or IL-2+IL-15-activated NK cells. (F) The coexpression pattern of KIR2DL5 with other receptors on NK cells from human PBMCs. The t-SNE plots were generated based on spectral flow cytometric data (n=3). In E, data are representative of 3 independent experiments with 3 different donors. P values were determined by 1-way ANOVA (A and B) or 2-tailed paired t test (C and D).

FIG. 10A-E: Allelic polymorphism affected PVR binding of KIR2DL5. (A) Flow cytometric analysis of PVR-Ig or CD112-Ig (open) or control hIg (shaded) binding to KIR2DL5/3T3. (B) Flow cytometric analysis of PVR-Ig binding to KIR2DL5/3T3 in the presence of increasing concentrations of F8B30. (C) KIR2DL5 bound to different sites of PVR from other receptors. PVR-Ig protein was preincubated with indicated His-tagged protein and then stained KIR2DL5/3T3 cells. FIG. 3 (see above) shows that PVR/3T3 cells were preincubated with DNAM-1-His, CD96-His, TIGIT-His, or TMIGD2-His (negative control) tag protein at the indicated concentrations and then stained by KIR2DL5-Ig fusion protein. (D) Flow cytometric analysis of PVR-Ig (open) or control hIg (shaded) binding on 3T3 cells expressing WT, DO-deleted, or D2-deleted KIR2DL5. Parental 3T3 cells were used as a negative control. (E) Flow cytometric analysis of PVR-Ig or control hIg (shaded) binding on 3T3 cells expressing different KIR2DL5 alleles. In A-E, data are representative of 2 independent experiments.

FIG. 11A-11B: KIR2DL5 inhibited NK cell function and mediated PVR⁺ tumor immune resistance. (A) PVR-KIR2DL5-mediated inhibitory synapse formation. Left: Representative imaging of cell conjugates acquired upon sorted KIR2DL5⁺ primary NK contact with control-YFP/Raji (top) or PVR-YFP/Raji (bottom), followed by staining with anti-KIR2DL5 mAbs and phalloidin. Scale bars: 10 m. Right: Intensity quantification of F-actin, YFP, and KIR2DL5 at the immunological synapses (IS) and the cell surface away from synapses (Non-IS) from KIR2DL5⁺ NK cell-Control Raji (n=25) and KIR2DL5⁺ NK-PVR/Raji (n=35) conjugates. (B) Lysis of scrambled control or PVR^{KO} A427 (top) or Jurkat (bottom) cells by sorted KIR2DL5⁺ primary NK cells in the presence of F8B30 or mIgG1 at indicated E/T ratios. In B, data are mean for duplicate measurements and representative of 3 independent experiments with 3 different donors. *P<0.05, **P<0.01, ***P<0.001, ****P<0.0001, by 2-tailed paired Student's t test (A), or multiple unpaired t test (B).

FIG. 12A-12E: KIR2DL5 ITIM and ITSM mediated NK cell inhibition and suppressed downstream signaling. (A) Tyrosine (Y) in ITIM and ITSM of KIR2DL5 was mutated to phenylalanine (F). The KIR2DL5⁻ primary NK cells were transduced with WT KIR2DL5 or the indicated mutants, and then examined for protein expression with F8B30 (open) or mIgG1 (shaded). NC, negative control. Data are representative of 2 independent experiments. (B and C) Transduced primary NK cells were treated with (+) or without (−) pervanadate (VO₄) for 5 minutes. Cell lysates were immunoprecipitated with anti-KIR2DL5 antibodies. Phospho-tyrosine (4G10), SHP-1, SHP-2, and total KIR2DL5 were detected by immunoblots (B). Quantification of p-Tyr, SHP-1, and SHP-2 association with WT or mutant KIR2DL5 in VO₄-treated NK cells (C). WCL, whole-cell lysates. (D) Representative imaging of cell conjugates acquired upon the indicated transduced primary NK and PVR/Raji cell contact followed by staining with anti-KIR2DL5 mAb and DAPI. Scale bars: 10 m. (E) Lysis of scramble control or PVR^{KO} A427 (top) and Jurkat (bottom) by WT or mutant KIR2DL5-transduced primary NK cells at the indicated E/T ratios. Data are mean for duplicate measurements and representative of 3 independent experiments with 3 different donors. In A, and B, data are representative of 3 independent experiments. *P<0.05, **P<0.01, ***P<0.001, ****P<0.0001, by 1-way ANOVA (C), or paired Student's t test (D).

FIG. 13: KIR2DL5⁺ immune cells infiltrated in various PVR⁺ human cancers. Representative images of the coexpression of KIR2DL5 and CD45 mRNA detected by RNAScope (left) and PVR protein expression detected by IHC (right) in the indicated cancer types. The gates in top right of the RNAScope images showed coexpression of KIR2DL5 (green) and CD45 (red) mRNA in indicated human cancers. Scale bars: 50 m for RNAScope images and 200 m for IHC images.

FIG. 14A-14H: KIR2DL5 blockade promoted NK-based antitumor immunity. (A and B) KIR2DL5 blockade enhanced NK cell function in vitro. Sorted KIR2DL5⁺ primary NK cells preincubated with mIgG1 or F8B30 were cocultured with A427 (A) or Jurkat (B) tumor cells at E/T of 2:1 and 5:1, respectively. Tumor cell lysis and the degranulation (CD107a) and cytokine production (IFN-γ and TNF-γ) of NK cells from different donors (n=6 for A427, n=4 for Jurkat) are shown. (C) Lysis of A427 and K562 cells by sorted primary KIR2DL5⁺ NK cells in the presence of indicated mAbs at indicated E/T ratios. Data are mean for duplicate measurements and representative of 3 independent experiments with 3 different donors. (D) Subcutaneous A427 tumor mode with sorted primary KIR2DL5⁺ NK cells. (D) Schematic of experimental design. Growth of A427 tumors. n=8 tumors per group (FIG. 6A, see above). Kaplan-Meier survival curves of mice (FIG. 6B, see above). (E-F) A427 lung metastasis model with sorted primary KIR2DL5⁺ NK cells. (E) Schematic of experimental design. (F and FIG. 6C, see above) Tumor growth was monitored by means of bioluminescence imaging. n=5 mice per group. Kaplan-Meier survival curves of mice (FIG. 6D, see above). (G-H) Jurkat metastasis model with sorted primary KIR2DL5⁺ NK cells. (G) Schematic of experimental design. (L and FIG. 6E, see above) Tumor growth was monitored by means of bioluminescence imaging. n=6 mice per group. Kaplan-Meier survival curves of mice (FIG. 6F, see above). In D, E, and G, data are representative of 2 independent experiments. P values were determined by 2-tailed paired Student's t test (A and B).

FIG. 15A-15E: Characterization of anti-KIR2DL5 specific monoclonal antibodies. (Related to FIGS. 8A-8D). (A) The protein sequence of KIR2DL5. The extracellular domain of KIR2DL5 was composed of tandem DO-D2 domains. The cytoplasmic tail of KIR2DL5 contained an immunoreceptor tyrosine-based inhibition motif (ITIM) and immunoreceptor tyrosine-based switch motif (ITSM). Domains were predicted and annotated based on UniProtKB (Q8N109). (B) The specificity of anti-KIR2DL5 mAbs. 3T3 cells transduced with indicated KIR family members were stained with 5 μg/ml of indicated anti-KIR2DL5 mAbs (open) or mIgG1 (shaded). (C, D) The affinity of anti-KIR2DL5 mAbs. C: Kinetic binding curves for F8B30 and B19C11. D: Data were acquired from kinetic binding curves detected by the Octet Red96 BLI instrument for indicated clones. (E) 3T3 cells transduced with DO-deleted (KIR2DL5 dD0) or D2-deleted KIR2DL5 (KIR2DL5 dD2) were stained with 5 μg/ml of indicated anti-KIR2DL5 mAbs. In B-E, data are representative of three independent experiments.

FIG. 16A-16B: Allelic polymorphism affected mAb recognition of KIR2DL5. (Related to FIGS. 8A-8D). (A) The binding of anti-KIR2DL5 mAbs (5 μg/ml, open) and mIgG1 (shaded) to the 3T3 cells expressing different KIR2DL5A and KIR2DL5B alleles. (B) The binding of anti-KIR2DL5 mAbs (5 μg/ml, open) and mIgG1 (shaded) to the 3T3 cells expressing different KIR2DL5 DO variants. In A and B, data are representative of two independent experiments.

FIG. 17A-17B: KIR2DL5 was predominantly expressed on mature NK cells. (Related to FIGS. 9A-9F). (A) Gating strategy for immune cell subsets in human PBMCs. (a) The major lymphocytes was gated based on the FCS-A and SSC-A; (b) Doublets were excluded based on plotted in SSC-H/SSC-A. (c) Live CD19⁻ cells were gated from single cells based on CD19 and Live/Dead blue staining; (d) CD3⁻ CD56⁺ cells were defined as NK cells; (e) From CD3⁺ CD56⁻ cells, γδ T were defined based on TCRγ/δ staining; (f) CD3⁺ TCRγ/δ⁻ cells were then divided into CD4⁺ T and CD8⁺ T subsets. (B) KIR2DL5A expression in human normal hematopoietic cells. Hierarchical differentiation tree was generated from BloodSpot database (https://servers.binf.ku.dk/bloodspot/?gene=KIR2DL5A&dataset=DMAP).

FIG. 18A-18C: Characterization of KIR2DL5 as a binding partner for PVR. (Related to FIGS. 10A-10F). (A) Flow cytometric analysis of PVR binding to KIR2DL5/3T3 or KIR2DL4/3T3 at increasing concentrations of PVR-Ig. (B) KIR2DL5-PVR interaction by intercellular conjugate assay. Left: Prelabeled KIR2DL5/3T3 and PVR/3T3 cells were co-incubated and then analyzed by flow cytometry. KIR3DL3/3T3+HHLA2/3T3 and KIR3DL3/3T3+PVR/3T3 co-incubation were used as a positive and negative control, respectively. Right: Summary of the intercellular conjugation of indicated groups. Data are mean±SEM from three independent experiments. P values by a one-way ANOVA. (C) Intercellular conjugate assay between KIR2DL5/3T3 and PVR/3T3 in the presence of indicated anti-KIR2LD5 mAbs. KIR3DL3/3T3+PVR/3T3 co-incubation was used as a negative control. In A and C, data are representative of three independent experiments.

FIG. 19A-19J: KIR2DL5 mediated PVR⁺ tumor immune resistance to NK cell cytotoxicity. (Related to FIGS. 5A-5D, and 11A-11B). (A) KIR2DL5⁺ primary NK cells were sorted from human PBMCs and cultured in vitro. The expression of KIR2DL5 was confirmed with F8B30 (open) or mIgG1 (shaded) by flow cytometry. (B) Expression of other immune receptors on KIR2DL5⁺ primary NK cells in A. Data are represented as means±SEM of six different donors. (C) Primary NK cells were transduced with empty vector (control NK) or KIR2DL5 (KIR2DL5/NK) and examined for KIR2DL5 expression with F8B30 (open) or mIgG1 (shaded). (D,E) The expression of activating or inhibitory ligands on A427 (D) and Jurkat (E). Cells was stained by the indicated markers (open) and isotype control (shaded). (F-H) Scrambled control and PVR^{KO} A427 (F), Jurkat (G) and K562 (H) cell lines were generated and examined for PVR expression with anti-PVR mAb (open) or isotype control (shaded). (I) Lysis of scrambled control or PVR^{KO} K562 cells by KIR2DL5⁺ primary NK cells or control KIR2DL5-NK cells at indicated E:T ratios. Data are mean for duplicate measurements and representative of three independent experiments with three different donors. (J) Control Raji and PVR/Raji cell lines were generated and examined for PVR expression with anti-PVR mAb (open) or isotype control (shaded). In A, CH and J, data are representative of three independent experiments. P values by a multiple unpaired t-test (I). **P<0.01, ****P<0.0001; ns, not significant.

FIG. 20A-20B: ERK1/2/p90RSK pathway was involved in KIR2DL5 downstream signaling. (Related to FIGS. 7A-7B, and 12A-12E). (A, B) A human phospho-kinase array of KIR2DL5⁺ primary NK cells after crosslinking with anti-CD16 and mIgG1 (CD16 alone), or anti-KIR2DL5 mAb F8B30 (CD16+KIR2DL5) for 2 minutes. A: Kinase spots with significantly different densities between two groups are indicated. B: Relative quantification of the phosphorylation level of indicated kinases. Data are representative of two independent experiments.

FIG. 21A-21C: KIR2DL5 was upregulated in solid and hematopoietic tumors. (Related to FIG. 13). (A) The mRNA expression of KIR2DL5, TIGIT, CD96, and DNAM-1 in human tumors versus corresponding normal tissues by analyzing indicated Gene Expression Omnibus (GEO) databases. (Breast cancer, n=43 versus 7; ATLL (adult T cell leukemia/lymphoma, n=12 versus 10; MCC (merkel cell carcinoma), n=27 versus 64 samples of human tumor versus normal tissues). Data are mean±SEM. (B) Analysis of KIR2DL5A mRNA expression in primary human AML (acute myeloid leukemia) across cytogenetic subtypes in comparison with normal hematopoietic cells. Hierarchical differentiation tree was generated from BloodSpot database (https:/servers.binf.ku.dk/bloodspot/?gene=KIR2DL5A&dataset=MERGED_AML). (C) Representative RNAScope images of positive staining for KIR2DL5 with KIR2DL5⁺ primary NK mixed with A427 cell slide and negative staining with KIR2DL5⁻ human PBMCs slide. Scale bar, 10 μm.

FIG. 22A-22E: KIR2DL5 blockade promoted NK-based anti-tumor immunity. (Related to FIGS. 6A-6F, and 14A-14H). (A) Tumor lysis and NK degranulation after co-culturing IL-2+ IL-15 stimulated primary NK cells with A427 cells in the presence of anti-TIGIT mAb or isotype control. (B) KIR2DL5⁺ primary NK cell degranulation after co-culturing with A427 or K562 cells in the presence of indicated mAbs at indicated E:T=2:1. (C-E) A427 subcutaneous tumor model in NSG-hIL15 mice. (C) Schematic of experimental design. NSG-hIL-15 mice were engrafted s.c. with A427 (3×10⁶/mouse) on day 0, followed by randomization on day 5 and i.t. treatment with KIR2DL5⁺ primary NK cells together with mIgG1 or F8B30 every three days for twice. (D) Growth of A427 tumors. n=6 tumors per group. (E) Tumor mass and images of each group at day 23. In A and B, data are means±SEM of three independent experiments with three or four different donors. P values by a two-tailed unpaired Student's t-test (A, E), one-way ANOVA (B), two-way ANOVA (D). s.c., subcutaneously; i.t., intratumorally. ns, not significant.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The natural killer cell protein KIR2DL5 is a type I transmembrane molecule containing an N-terminal signal peptide, an ectodomain composed of tandem D0-D2 domains, a transmembrane region, and a cytoplasmic tail with an immunoreceptor tyrosine-based inhibition motif (ITIM) and immunoreceptor tyrosine-based switch motif (ITSM). The amino acid sequence of KIR2DL5 is set forth in SEQ ID NO:1.

SEQ ID NO:1: MSLMVISMACVGFFLLQGAWTHEGGQDKPLLSAWPSAVVPRGGHVTLLCRSRLGFTIF SLYKEDGVPVPELYNKIFWKSILMGPVTPAHAGTYRCRGSHPRSPIEWSAPSNPLVIVVT GLFGKPSLSAQPGPTVRTGENVTLSCSSRSSFDMYHLSREGRAHEPRLPAVPSVNGTFQA DFPLGPATHGGTYTCFGSLHDSPYEWSDPSDPLLVSVTGNSSSSSSSPTEPSSKTGIRRHL HILIGTSVAIILFIILFFFLLHCCCSNKKNAAVMDQEPAGDRTVNREDSDDQDPQEVTYAQ LDHCVFTQTKITSPSQRPKTPPTDTTMYMELPNAKPRSLSPAHKHHSQALRGSSRETTAL SQNRVASSHVPAAGI (1-21: signal peptide; 22-239: extracellular domain; 42-102: DO; 137-200: D2; 240-259: transmembrane; 260-374: cytoplasmic tail; 296-301: ITIM; 326-331: ITSM; see also NP_065396.1).

KIR2DL5 has NCBI Accession Number and Ensembl Gene Number of NG_005994.1 (NM_020535.3, NP_065396.1) and ENSG00000274143.1, respectively. The NCBI Accession number(s) and Ensembl Gene Number(s) provided herein were accessed on Oct. 28, 2022.

KIR2DL5 was recently identified as a binding partner for poliovirus receptor (PVR) via a high-throughput in vitro screen of IgG superfamily (IgSF) (Husain 2019; Wojtowicz 2020). However, the biology of the KIR2DL5-PVR pathway is largely unknown (Beziat 2017).

As disclosed herein, KIR2DL5 is an inhibitory receptor of the immune system. Specifically, it was found that (1) KIR2DL5 and the PVR receptors TIGIT, DNAM-1, and CD96 can simultaneously bind to nonidentical sites on PVR; (2) KIR2DL5 is expressed on the surface of human adaptive immune cells and innate immune cells; (3) KIR2DL5 inhibits NK cell function and mediates PVR+ tumor immune resistance; and (5) KIR2DL5 blockade promotes anti-tumor immunity. Based on these results, methods and compositions are provided herein for increasing immune cell activity by inhibiting KIR2DL5 expression and/or activity, and for decreasing immune cell activity by increasing KIR2DL5 expression and/or activity.

KIR2DL5 is polymorphic and is represented by 2DL5A*001 and 2DL5A*005 (Cisneros 2016). While most KIR2DL5B alleles are epigenetically silent because of a distinctive substitution in a promoter RUNX binding site, 2DL5B*003 and 2DL5B*00602 alleles with intact RUNX binding sites are predicted to be transcribed and expressed on the cell surface (Du 2008). These 2 alleles have an identical DO domain to KIR2DL5A*001 through which F8B30 recognized KIR2DL5, contains only 4 polymorphic sites: T46S, R52H, G97S, and P112S (IPD-KIR Database, Release 2.9.0) (Robinson 2010).

### Definitions

The terms “treat,” “treating,” and “treatment” as used herein with regard to a condition refer to alleviating the condition partially or entirely; slowing the progression or development of the condition; eliminating, reducing, or slowing the development of one or more symptoms associated with the condition; or increasing progression-free or overall survival of the condition.

The terms “prevent,” “preventing,” and “prevention” as used herein with regard to a condition refers to averting the onset of the condition or decreasing the likelihood of occurrence or recurrence of the condition, including in a subject that may be predisposed to the condition but has not yet been diagnosed as having the condition.

The term “infectious disease” may refer to any disease caused by an infectious organism such as a virus, bacteria, parasite, and/or fungus.

The term “antibody” as used herein refers to an immunoglobulin molecule or an immunologically active portion thereof that binds to a specific antigen, e.g., a cancer cell antigen, viral antigen, or microbial antigen. In those embodiments where the targeting moiety is an antibody and the antibody is a full-length immunoglobulin molecule, the antibody comprises two heavy chains and two light chains, with each heavy and light chain containing three complementary determining regions (CDRs). In those embodiments where the targeting moiety is an antibody and the antibody is an immunologically active portion of an immunoglobulin molecule, the antibody may be, for example, a Fab, Fab′, Fv, F(ab′)2, disulfide-linked Fv, scFv, single domain antibody (dAb), diabody, triabody, tetrabody, or linear antibody. Antibodies used as targeting moieties may be, for example, natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen.

A “subject” as used herein refers to a mammalian subject, preferably a human.

Based on the results disclosed herein showing that KIR2DL5 inhibits human immune cell function, provided herein in certain embodiments are methods of increasing human immune cell function in a subject by decreasing KIR2DL5 expression and/or activity. Since increased immune cell function results in increased identification and removal of pathogens, methods are further provided for treating infectious disease in a subject by decreasing KIR2DL5 expression and/or activity. Similarly, since increased immune cell function may result in increased cancer cell killing, methods are provided for treating cancer in a subject by decreasing KIR2DL5 expression and/or activity.

In certain embodiments of the methods provided herein, KIR2DL5 activity is decreased in a subject by administering one or more agents that prevent or reduce KIR2DL5 binding to PVR. In certain of these embodiments, the agents bind KIR2DL5 and prevent or reduce its binding interaction with PVR, for example by binding KIR2DL5 at or near its binding site for PVR. In other embodiments, the agents bind PVR and prevent or reduce its binding interaction with KIR2DL5, for example by binding PVR at or near its binding site for KIR2DL5. In certain embodiments, agents that decrease KIR2DL5 activity by binding PVR and preventing or reducing KIR2DL5/PVR binding also block binding of PVR to one or more of its other known receptors, including TIGIT, DNAM-1, and CD96. In other embodiments, the agents prevent or reduce binding between KIR2DL5 and PVR while allowing PVR to bind one or more of its other known receptors. In certain embodiments of the methods provided herein, the agents that prevent or reduce KIR2DL5 binding to PVR are peptides, polypeptides, or small molecules. Suitable polypeptides include, but are not limited to, antibodies that specifically bind KIR2DL5 or PVR, truncated forms of KIR2DL5 or PVR (e.g., extracellular domain of KIR2DL5 or PVR or a portion thereof), and fusion polypeptides comprising antibodies or truncated forms of KIR2DL5 or PVR.

In certain embodiments of the methods provided herein, KIR2DL5 activity and/or expression is decreased in a subject by administering one or more agents that inhibit one or more pathways that upregulate KIR2DL5 expression. This inhibition may occur at any step in the pathway, for example by inhibiting the interaction between a surface receptor and its ligand, inhibiting the interaction between two or more intracellular proteins, blocking a KIR2DL5 promoter region, or the like.

In certain embodiments of the methods provided herein, KIR2DL5 activity and/or expression is decreased in a subject by altering a nucleotide sequence in the KIR2DL5 gene or one or more of its corresponding regulatory domains, e.g., promoters or enhancers. For example, in certain embodiments one or more nucleotide substitutions, insertions, or deletions may be introduced into the KIR2DL5 gene or its corresponding regulatory domains using a CRISPR/Cas (e.g., CRISPR/Cas9) system.

In some embodiments, methods are provided for treating an infectious disease in a subject in need thereof by decreasing KIR2DL5 expression and/or activity as disclosed herein. In certain of these embodiments, the infectious disease is caused by a pathogen. The pathogen can be one or more of a virus, bacterium, prion, fungus, and parasite. In some embodiments, the virus is selected from the group consisting of human immunodeficiency viruses, influenza viruses, papillomaviruses, coronaviruses, hepatitis viruses, or herpesviruses. In some embodiments, the bacterium is Mycobacterium tuberculosis. In certain embodiments, the fungus is Pneumocystis jirovecii (PJP).

In some embodiments, methods are provided for treating cancer in a subject in need thereof by decreasing KIR2DL5 expression and/or activity as disclosed herein. In certain of these embodiments, the cancer is selected from the group consisting of chronic lymphocytic leukemia (CLL), acute leukemia, acute lymphoid leukemia (ALL), B-cell acute lymphoid leukemia (B-ALL), T-cell lymphoma, B-cell lymphoma, T-cell acute lymphoid leukemia (T-ALL), chronic myelogenous leukemia (CML), B-cell prolymphocytic leukemia, T-cell lymphoma, Hodgkin's disease, B-cell non-Hodgkin's lymphoma. blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenström macroglobulinemia, or preleukemia. In other embodiments, the cancer the cancer is selected from the group consisting of colon cancer, rectal cancer, renal-cell carcinoma, liver cancer, lung cancer, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers, combinations of the cancers, and metastatic lesions of the cancers. In other embodiments, the cancer is a human hematologic malignancy.

In certain embodiments, the human hematologic malignancy is selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In certain embodiments of the methods provided herein, the agents that decrease KIR2DL5 activity and/or expression are antibodies, immunogenic fragments thereof, or antibody fragments thereof that specifically bind KIR2DL5, or fusion proteins comprising such antibodies. In certain of these embodiments, the antibodies are monoclonal antibodies. In certain embodiments, the antibodies are chimeric antibodies, humanized antibodies, or fully human antibodies.

In certain embodiments of the methods provided herein wherein the agents that decrease KIR2DL5 activity and/or expression are KIR2DL5 antibodies, immunogenic fragments thereof, or antibody fragments thereof, which comprise a variable heavy (VH) chain sequence comprising one or more of the CDR sequences of antibody B2A18 disclosed herein, i.e., residues 45-54, 69-85, and 116-131 of SEQ ID NO:3. In certain of these embodiments, the VH chain comprises, consists of, or consists essentially of the amino acid of SEQ ID NO:3 or an amino acid sequence at least 80%, 85%, 90%, 95%, 96%, 97%, 98%, or 99% identical to SEQ ID NO:3. In certain embodiments, the VH chain is encoded by the nucleotide sequence of SEQ ID NO:2 or a nucleotide sequence at least 80%, 85%, 90%, 95%, 96%, 97%, 98%, or 99% identical to SEQ ID NO:2.

In certain embodiments of the methods provided herein wherein the agents that decrease KIR2DL5 activity and/or expression are KIR2DL5 antibodies, immunogenic fragments thereof, or antibody fragments thereof, which comprise a variable light (VL) chain sequence comprising one or more of the CDR sequences of antibody B2A18 disclosed herein, i.e., residues 24-32, 50-56, and 89-97 of SEQ ID NO:5. In certain of these embodiments, the VL chain comprises, consists of, or consists essentially of the amino acid of SEQ ID NO:5 or an amino acid sequence at least 80%, 85%, 90%, 95%, 96%, 97%, 98%, or 99% identical to SEQ ID NO:5. In certain embodiments, the VL chain is encoded by the nucleotide sequence of SEQ ID NO:4 or a nucleotide sequence at least 80%, 85%, 90%, 95%, 96%, 97%, 98%, or 99% identical to SEQ ID NO:4.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:3 or an amino acid sequence at least 80%, 85%, 90%, 95%, 96%, 97%, 98%, or 99% identical to SEQ ID NO:3 and a VL chain comprising, consisting of, or consisting essentially of the amino acid of SEQ ID NO:5 or an amino acid sequence at least 80%, 85%, 90%, 95%, 96%, 97%, 98%, or 99% identical to SEQ ID NO:5.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody B7B23 disclosed herein, i.e., residues 45-54, 69-85, and 116-125 of SEQ ID NO:7. In certain of these embodiments, the VH chain comprises, consists of, or consists essentially of the amino acid of SEQ ID NO:7 or an amino acid sequence at least 80%, 85%, 90%, 95%, 96%, 97%, 98%, or 99% identical to SEQ ID NO:7. In certain embodiments, the VH chain is encoded by the nucleotide sequence of SEQ ID NO:6 or a nucleotide sequence at least 80%, 85%, 90%, 95%, 96%, 97%, 98%, or 99% identical to SEQ ID NO:6.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody B7B23 disclosed herein, i.e., residues 47-63, 79-85, and 118-126 of SEQ ID NO:9. In certain of these embodiments, the VL chain comprises, consists of, or consists essentially of the amino acid of SEQ ID NO:9 or an amino acid sequence at least 80%, 85%, 90%, 95%, 96%, 97%, 98%, or 99% identical to SEQ ID NO:9. In certain embodiments, the VL chain is encoded by the nucleotide sequence of SEQ ID NO:8 or a nucleotide sequence at least 80%, 85%, 90%, 95%, 96%, 97%, 98%, or 99% identical to SEQ ID NO:8.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:7 or an amino acid sequence at least 80%, 85%, 90%, 95%, 96%, 97%, 98%, or 99% identical to SEQ ID NO:7 and a VL chain comprising, consisting of, or consisting essentially of the amino acid of SEQ ID NO:9 or an amino acid sequence at least 80%, 85%, 90%, 95%, 96%, 97%, 98%, or 99% identical to SEQ ID NO:9.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody B11B4 disclosed herein, i.e., residues 45-54, 69-87, and 116-127 of SEQ ID NO:11. In certain of these embodiments, the VH chain comprises, consists of, or consists essentially of the amino acid of SEQ ID NO:11 or an amino acid sequence at least 80%, 85%, 90%, 95%, 96%, 97%, 98%, or 99% identical to SEQ ID NO:11. In certain embodiments, the VH chain is encoded by the nucleotide sequence of SEQ ID NO:10 or a nucleotide sequence at least 80%, 85%, 90%, 95%, 96%, 97%, 98%, or 99% identical to SEQ ID NO:10.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody B11B4 disclosed herein, i.e., residues 46-60, 76-82, and 115-123 of SEQ ID NO:13. In certain of these embodiments, the VL chain comprises, consists of, or consists essentially of the amino acid of SEQ ID NO:13 or an amino acid sequence at least 80%, 85%, 90%, 95%, 96%, 97%, 98%, or 99% identical to SEQ ID NO:13. In certain embodiments, the VL chain is encoded by the nucleotide sequence of SEQ ID NO:12 or a nucleotide sequence at least 80%, 85%, 90%, 95%, 96%, 97%, 98%, or 99% identical to SEQ ID NO:12.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:11 or an amino acid sequence at least 80%, 85%, 90%, 95%, 96%, 97%, 98%, or 99% identical to SEQ ID NO:11 and a VL chain comprising, consisting of, or consisting essentially of the amino acid of SEQ ID NO:13 or an amino acid sequence at least 80%, 85%, 90%, 95%, 96%, 97%, 98%, or 99% identical to SEQ ID NO:13.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody B19C11 disclosed herein, i.e., residues 44-54, 69-84, and 115-129 of SEQ ID NO:15. In certain of these embodiments, the VH chain comprises, consists of, or consists essentially of the amino acid of SEQ ID NO:15 or an amino acid sequence at least 80%, 85%, 90%, 95%, 96%, 97%, 98%, or 99% identical to SEQ ID NO:15. In certain embodiments, the VH chain is encoded by the nucleotide sequence of SEQ ID NO:14 or a nucleotide sequence at least 80%, 85%, 90%, 95%, 96%, 97%, 98%, or 99% identical to SEQ ID NO:14.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody B19C11 disclosed herein, i.e., residues 24-38, 54-60, and 93-101 of SEQ ID NO:17. In certain of these embodiments, the VL chain comprises, consists of, or consists essentially of the amino acid of SEQ ID NO:17 or an amino acid sequence at least 80%, 85%, 90%, 95%, 96%, 97%, 98%, or 99% identical to SEQ ID NO:17. In certain embodiments, the VL chain is encoded by the nucleotide sequence of SEQ ID NO:16 or a nucleotide sequence at least 80%, 85%, 90%, 95%, 96%, 97%, 98%, or 99% identical to SEQ ID NO:16.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:15 or an amino acid sequence at least 80%, 85%, 90%, 95%, 96%, 97%, 98%, or 99% identical to SEQ ID NO:15 and a VL chain comprising, consisting of, or consisting essentially of the amino acid of SEQ ID NO:17 or an amino acid sequence at least 80%, 85%, 90%, 95%, 96%, 97%, 98%, or 99% identical to SEQ ID NO:17.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody B33C12 disclosed herein, i.e., residues 45-54, 69-87, and 116-127 of SEQ ID NO:19. In certain of these embodiments, the VH chain comprises, consists of, or consists essentially of the amino acid of SEQ ID NO:19 or an amino acid sequence at least 80%, 85%, 90%, 95%, 96%, 97%, 98%, or 99% identical to SEQ ID NO:19. In certain embodiments, the VH chain is encoded by the nucleotide sequence of SEQ ID NO:18 or a nucleotide sequence at least 80%, 85%, 90%, 95%, 96%, 97%, 98%, or 99% identical to SEQ ID NO:18.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody B33C12 disclosed herein, i.e., residues 44-58, 74-80, and 113-121 of SEQ ID NO:21. In certain of these embodiments, the VL chain comprises, consists of, or consists essentially of the amino acid of SEQ ID NO:21 or an amino acid sequence at least 80%, 85%, 90%, 95%, 96%, 97%, 98%, or 99% identical to SEQ ID NO:21. In certain embodiments, the VL chain is encoded by the nucleotide sequence of SEQ ID NO:20 or a nucleotide sequence at least 80%, 85%, 90%, 95%, 96%, 97%, 98%, or 99% identical to SEQ ID NO:20.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:19 or an amino acid sequence at least 80%, 85%, 90%, 95%, 96%, 97%, 98%, or 99% identical to SEQ ID NO:19 and a VL chain comprising, consisting of, or consisting essentially of the amino acid of SEQ ID NO:21 or an amino acid sequence at least 80%, 85%, 90%, 95%, 96%, 97%, 98%, or 99% identical to SEQ ID NO:21.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody E12B11 disclosed herein, i.e., residues 45-54, 69-85, and 116-131 of SEQ ID NO:23. In certain of these embodiments, the VH chain comprises, consists of, or consists essentially of the amino acid of SEQ ID NO:23 or an amino acid sequence at least 80%, 85%, 90%, 95%, 96%, 97%, 98%, or 99% identical to SEQ ID NO:23. In certain embodiments, the VH chain is encoded by the nucleotide sequence of SEQ ID NO:22 or a nucleotide sequence at least 80%, 85%, 90%, 95%, 96%, 97%, 98%, or 99% identical to SEQ ID NO:22.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody E12B11 disclosed herein, i.e., residues 24-34, 50-56, and 89-97 of SEQ ID NO:25. In certain of these embodiments, the VL chain comprises, consists of, or consists essentially of the amino acid of SEQ ID NO:25 or an amino acid sequence at least 80%, 85%, 90%, 95%, 96%, 97%, 98%, or 99% identical to SEQ ID NO:25. In certain embodiments, the VL chain is encoded by the nucleotide sequence of SEQ ID NO:24 or a nucleotide sequence at least 80%, 85%, 90%, 95%, 96%, 97%, 98%, or 99% identical to SEQ ID NO:24.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:23 or an amino acid sequence at least 80%, 85%, 90%, 95%, 96%, 97%, 98%, or 99% identical to SEQ ID NO:23 and a VL chain comprising, consisting of, or consisting essentially of the amino acid of SEQ ID NO:25 or an amino acid sequence at least 80%, 85%, 90%, 95%, 96%, 97%, 98%, or 99% identical to SEQ ID NO:25.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody F8B30 disclosed herein, i.e., residues 45-54, 69-85, and 116-125 of SEQ ID NO:27. In certain of these embodiments, the VH chain comprises, consists of, or consists essentially of the amino acid of SEQ ID NO:27 or an amino acid sequence at least 80%, 85%, 90%, 95%, 96%, 97%, 98%, or 99% identical to SEQ ID NO:27. In certain embodiments, the VH chain is encoded by the nucleotide sequence of SEQ ID NO:26 or a nucleotide sequence at least 80%, 85%, 90%, 95%, 96%, 97%, 98%, or 99% identical to SEQ ID NO:26.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody F8B30 disclosed herein, i.e., residues 24-34, 50-56, and 89-97 of SEQ ID NO:29. In certain of these embodiments, the VL chain comprises, consists of, or consists essentially of the amino acid of SEQ ID NO:29 or an amino acid sequence at least 80%, 85%, 90%, 95%, 96%, 97%, 98%, or 99% identical to SEQ ID NO:29. In certain embodiments, the VL chain is encoded by the nucleotide sequence of SEQ ID NO:28 or a nucleotide sequence at least 80%, 85%, 90%, 95%, 96%, 97%, 98%, or 99% identical to SEQ ID NO:28.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:27 or an amino acid sequence at least 80%, 85%, 90%, 95%, 96%, 97%, 98%, or 99% identical to SEQ ID NO:27 and a VL chain comprising, consisting of, or consisting essentially of the amino acid of SEQ ID NO:29 or an amino acid sequence at least 80%, 85%, 90%, 95%, 96%, 97%, 98%, or 99% identical to SEQ ID NO:29.

In certain embodiments of the methods provided herein wherein the agents that decrease KIR2DL5 activity and/or expression are KIR2DL5 antibodies, immunogenic fragments thereof, or antibody fragments thereof, which comprise a VH chain sequence comprising one or more of the CDR sequences of antibody G11B22 disclosed herein, i.e., residues 45-54, 69-85, and 116-131 of SEQ ID NO:31. In certain of these embodiments, the VH chain comprises, consists of, or consists essentially of the amino acid of SEQ ID NO:31 or an amino acid sequence at least 80%, 85%, 90%, 95%, 96%, 97%, 98%, or 99% identical to SEQ ID NO:31. In certain embodiments, the VH chain is encoded by the nucleotide sequence of SEQ ID NO:30 or a nucleotide sequence at least 80%, 85%, 90%, 95%, 96%, 97%, 98%, or 99% identical to SEQ ID NO:30.

In certain embodiments of the methods provided herein wherein the agents that decrease KIR2DL5 activity and/or expression are KIR2DL5 antibodies, immunogenic fragments thereof, or antibody fragments thereof, which comprise a VL chain sequence comprising one or more of the CDR sequences of antibody G11B22 disclosed herein, i.e., residues 24-34, 50-56, and 89-97 of SEQ ID NO:33. In certain of these embodiments, the VL chain comprises, consists of, or consists essentially of the amino acid of SEQ ID NO:33 or an amino acid sequence at least 80%, 85%, 90%, 95%, 96%, 97%, 98%, or 99% identical to SEQ ID NO:33. In certain embodiments, the VL chain is encoded by the nucleotide sequence of SEQ ID NO:32 or a nucleotide sequence at least 80%, 85%, 90%, 95%, 96%, 97%, 98%, or 99% identical to SEQ ID NO:32.

In certain embodiments of the methods provided herein wherein the agents that decrease KIR2DL5 activity and/or expression are KIR2DL5 antibodies, immunogenic fragments thereof, or antibody fragments thereof, which comprise a VH chain comprising, consisting of, or consisting essentially of the amino acid of SEQ ID NO:31 or an amino acid sequence at least 80%, 85%, 90%, 95%, 96%, 97%, 98%, or 99% identical to SEQ ID NO:31 and a VL chain comprising, consisting of, or consisting essentially of the amino acid of SEQ ID NO:33 or an amino acid sequence at least 80%, 85%, 90%, 95%, 96%, 97%, 98%, or 99% identical to SEQ ID NO:33.

Based on the results disclosed herein showing that KIR2DL5 inhibits human immune cell function, further provided herein in certain embodiments are methods of decreasing human immune cell function in a subject by increasing KIR2DL5 expression and/or activity. Since immune cell hyperactivity may be associated with autoimmune disease, methods are further provided for treating an autoimmune disease in a subject by increasing KIR2DL5 expression and/or activity. Similarly, decreasing immune cell activity may be helpful in the context of transplantation, where it is desirable to suppress the immune system to prevent transplant rejection. Accordingly, also provided are methods for decreasing transplant rejection by increasing KIR2DL5 expression and/or activity.

In certain embodiments of the methods provided herein, KIR2DL5 activity and/or expression is increased in a subject by administering one or more agents that are KIR2DL5 or PVR agonists. In certain embodiments, the agents increase the binding affinity of KIR2DL5 for PVR. In certain embodiments, the agents mimic KIR2DL5 by binding and activating PVR.

In certain embodiments of the methods provided herein, KIR2DL5 activity and/or expression is increased by administering one or more agents that increase the activity of one or more pathways that upregulate KIR2DL5 expression. This increased activity may occur at any step in the pathway, for example by activating a surface receptor, increasing the interaction between two or more intracellular proteins, activating a KIR2DL5 promoter region, or the like.

Suitable agents for increasing KIR2DL5 activity and/or expression included, but are not limited to, peptides, polypeptides (e.g., antibodies or fusion proteins), or small molecules.

In some embodiments, methods are provided for treating an autoimmune disorder in a subject in need thereof by increasing KIR2DL5 activity and/or expression as disclosed herein. In certain of these embodiments, the autoimmune disease or disorder is selected from the group consisting of acute disseminated encephalomyelitis (ADEM), alopecia areata, antiphospholipid syndrome, autoimmune cardiomyopathy, autoimmune hemolytic anemia, autoimmune hepatitis, autoimmune inner ear disease, autoimmune lipoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Behçet's disease, celiac disease, Chagas disease, cold agglutinin disease, Crohn's disease, dermatomyositis, diabetes mellitus type 1, eosinophilic fasciitis, gastrointestinal pemphigoid, Goodpasture's syndrome, Grave's syndrome, Guillain-Barré syndrome, Hashimoto's encephalopathy, Hashimoto's thyroiditis, lupus erythematosus, Miller-Fisher syndrome, mixed connective tissue disease, myasthenia gravis, pemphigus vulgaris, pernicious anemia, polymyositis, psoriasis, psoriatic arthritis, relapsing polychondritis, rheumatoid arthritis, rheumatic fever, Sjögren's syndrome, temporal arteritis, transverse myelitis, ulcerative colitis, undifferentiated connective tissue disease, vasculitis, and Wegener's granulomatosis. In certain embodiments, the condition is adult rheumatoid arthritis.

In some embodiments, methods are provided for decreasing transplant rejection in a subject in need thereof by increasing KIR2DL5 activity and/or expression. In certain of these embodiments, the transplant is selected from a stem cell transplant or a bone marrow transplant. In some embodiments, the transplant is selected from the group consisting of a kidney transplant, a lung transplant, a heart transplant, a pancreas transplant, a cornea transplant, or a liver transplant. In some embodiments, KIR2DL5 activity and/or expression is increased in advance of transplantation, i.e., one or more hours, days, or weeks prior to transplantation; at the time of transplantation; and/or after transplantation.

Provided herein in certain embodiments are agents for use in the disclosed methods, namely agents that either decrease or increase KIR2DL5 activity and/or expression. These agents may be small molecules, peptides, or polypeptides, including antibodies and fusion proteins, as disclosed herein. Also provided herein are formulations, including pharmaceutical formulations, comprising such agents, as well as kits comprising any of the disclosed agents or formulations.

The foregoing and the following working examples are merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### EXAMPLES

### Example 1: Characterization of KIR2DL5 Binding

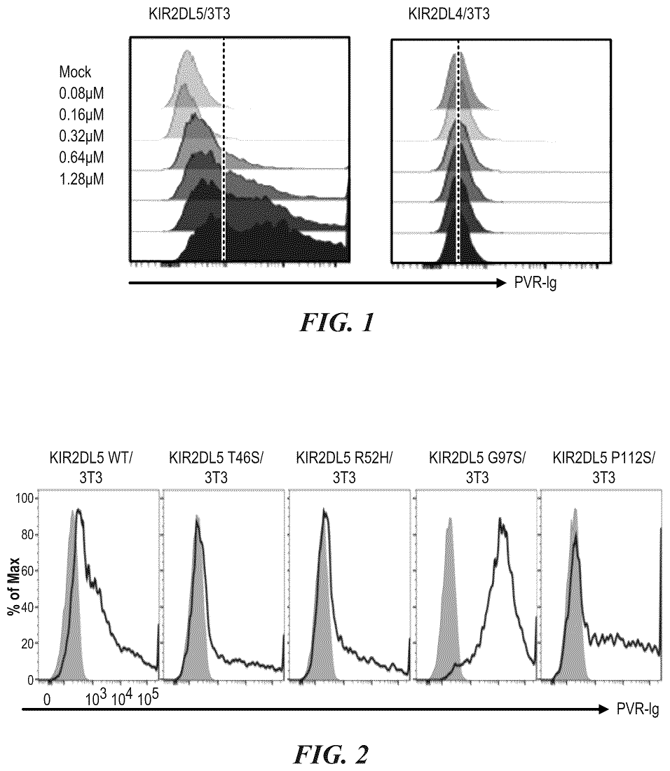

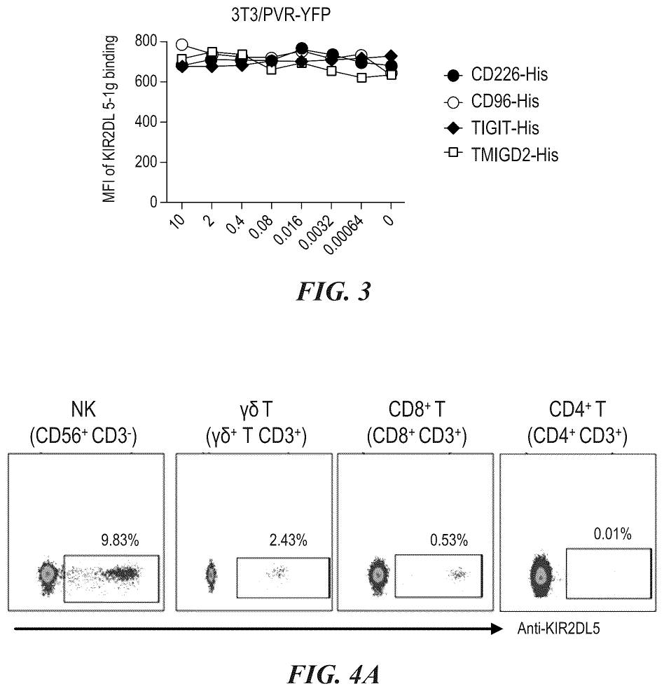

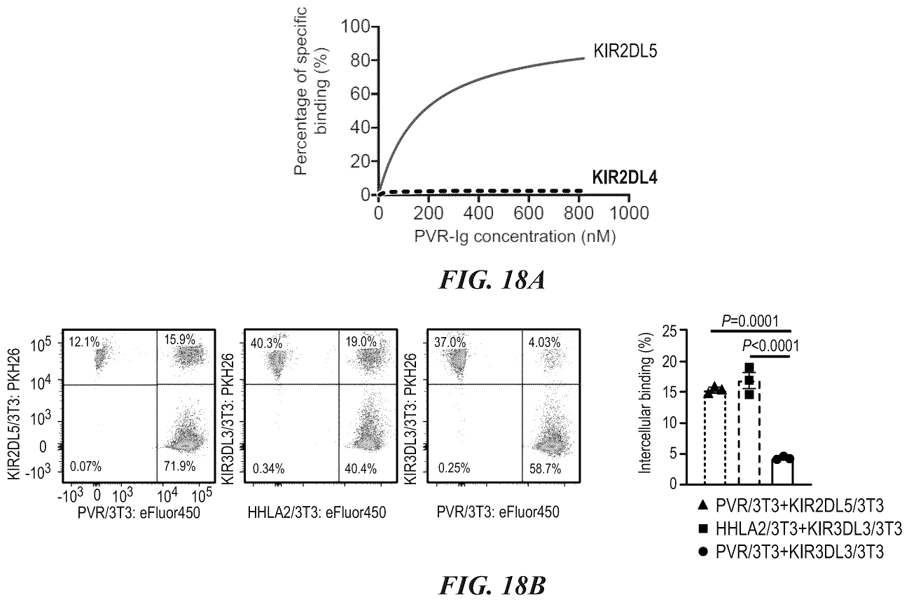

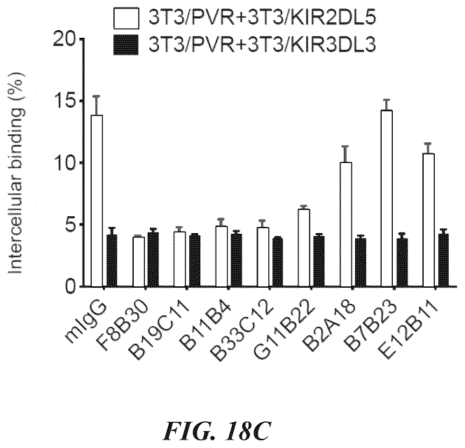

In this example, it was determined that PVR-Ig protein binds KIR2DL5 expressed on 3T3 cells in a dose-dependent manner, but does not bind KIR2DL4, a close homolog of KIR2DL5 (FIGS. 1 and 18A). PVR-Ig protein bound not only to wild-type KIR2DL5, but also to four KIR2DL5 DO variants: T46S, R52H, G97S, and P112S (FIG. 2). TIGIT, DNAM-1, and CD96 are known receptors for PVR and share a common binding site on PVR4. It was determined that KIR2DL5 and these three PVR receptors simultaneously bind non-identical sites on PVR (FIG. 3).

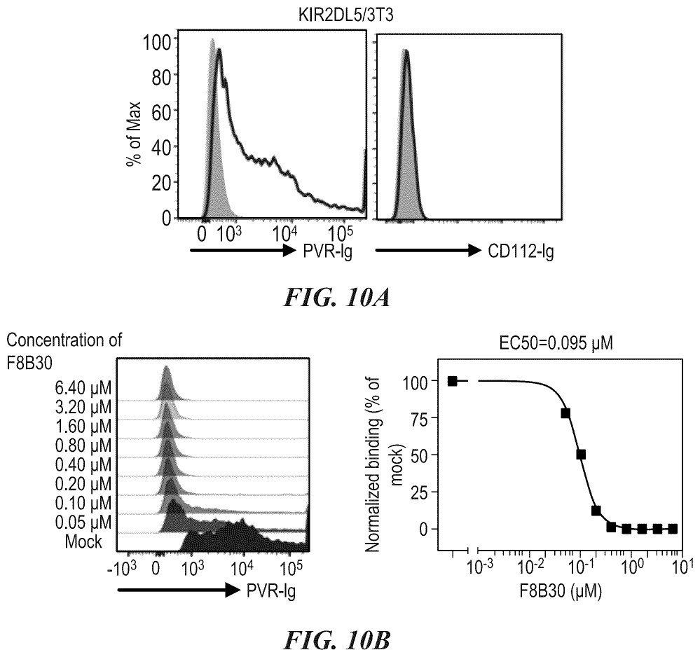

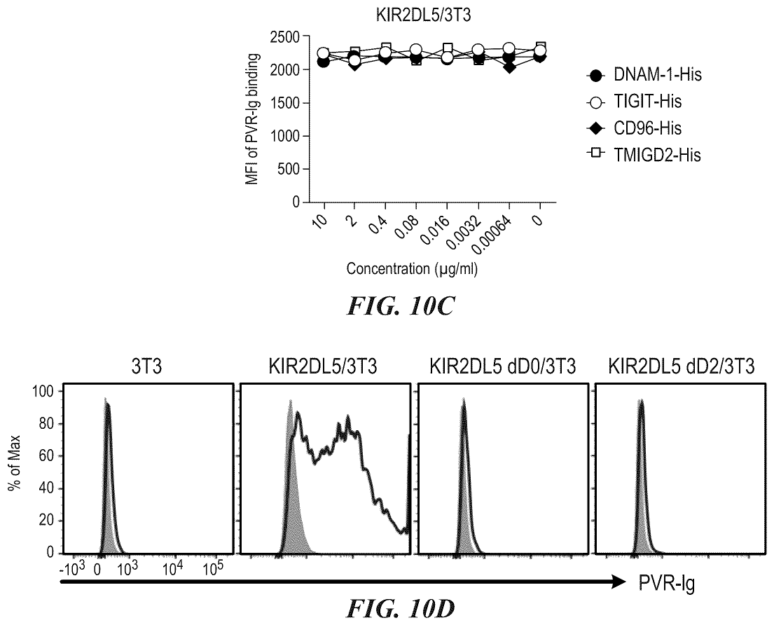

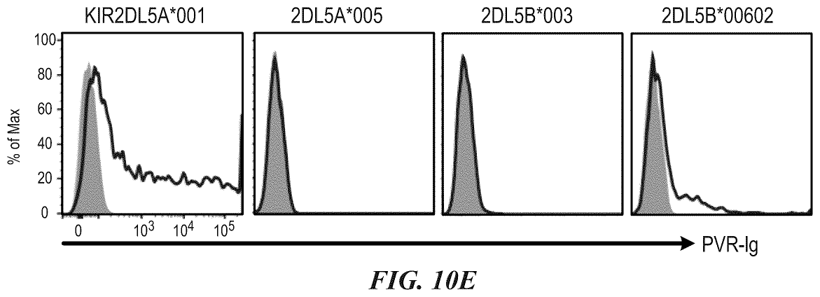

Specifically, a cell-based binding assay was performed by incubating PVR-Ig fusion protein with KIR2DL5- or KIR2DL4-expressing 3T3 cells. Conversely, KIR2DL5 was selectively bound by PVR, but not by CD112 (also known as nectin-2), another ligand for TIGIT and DNAM-1 in the nectin/nectin-like family (FIG. 10A). Furthermore, anti-KIR2DL5 mAb F8B30 blocked KIR2DL5-PVR interaction (EC₅₀=0.095 μM) (FIG. 10B). The specificity of KIR2DL5 binding to PVR was also evidenced by an intercellular interaction assay, in which 3T3 cells expressing PVR interacted with 3T3 cells expressing KIR2DL5, but not with cells expressing KIR3DL3 (FIG. 18B), a newly identified inhibitory receptor of HHLA2 (Zang, 2022; Wei 2021; Bhatt 2021). FIGS. 18B and 18C show that (i) KIR3DL3/3T3 cells interacted with HHLA2/3T3 but not with PVR/3T3 cells; and (ii) the interaction between KIR2DL5/3T3 and PVR/3T3 was blocked anti-KIR2DL5 mAbs of the present technology.

In competition studies, DNAM-1, TIGIT, and CD96 receptors did not block the interaction of PVR with KIR2DL5 (FIG. 10C), indicating that KIR2DL5 bound to PVR through a nonidentical site compared with other PVR receptors.

The deletion of either DO or D2 alone abrogated binding to PVR (FIG. 10D), suggesting that both DO and D2 domains contribute to the KIR2DL5-PVR interaction. Compared with the solid binding for 2DL5A*001, PVR weakly bound to cell surface-expressed 2DL5B*00602 but not 2DL5A*005 or 2DL5B*003 (FIG. 10E). As shown in FIG. 2, a serine substitution for glycine-97 in the DO domain (G97S) significantly enhanced the PVR-Ig binding to KIR2DL5, whereas the other DO variants showed a minor effect on PVR-KIR2DL5 binding.

### Example 2: Generation of KIR2DL5 Antibodies

A KIR2DL5 DO-Ig fusion protein was generated by fusing the KIR2DL5 DO coding region (H22-A128) to a human IgG1 Fc tag, and a KIR2DL5 DO-D2-Ig fusion protein was generated by fusing the KIR2DL5 D0D2 coding region (H22-H240) to a human IgG1 Fc tag using previously reported methods (Zhao 2013). The fusion proteins were expressed in a S2 system and then purified. Mice were immunized with KIR2DL5 DO(H22-A128)-Ig fusion protein and hybridomas were generated by standard techniques from splenocytes fused to NSO myeloma cells.

The VH and VL sequences of eight mAb clones are set forth in Table 1 (CDRs underlined, FRs italicized. Binding affinity as determined by biolayer interferometry for these eight clones is set forth in Table 2.

TABLE 1
B2A18

| | |
|---|---|
| VH chain | atgggttgggtgtggaccttgctattcctgatggcagctgcccaaagtatccaagcacagatccagttggtgcagtctgga |
| DNA | cctgaggtgaagaagcctggagagacagtcaagatctcctgcaaggcttctgggtacaccttcacaaagtatggaatga |
| | actgggtgaagcaggctccaggaaagggtttaaagtggatgggctggataaacaccaacactggagagtcaacatatg |
| | ctgaagacttcaagggacggtttgccttctctttggaaacctctgccaacactgcctttttgcagatcaacaacctcaaaaa |
| | tgaggacacggctgcatatttctgtgcaagatggggcccatacggtagtagcctttactatggtatggactactggggtcaa |
| | ggaacctcagtcaccgtctct (SEQ ID NO: 2; CDRH1: 133-162; CDRH2: 205-255; CDRH3: |
| | 346-393) |
| VH chain | MGWVWTLLFLMAAAQSIQAQIQLVQSGPEVKKPGETVKISCKASGYTFTKYG |
| AA | MNWVKQAPGKGLKWMGWINTNTGESTYAEDFKGRFAFSLETSANTAFLQINN |
| | LKNEDTAAYFCARWGPYGSSLYYGMDYWGQGTSVTVS (SEQ ID NO: 3; |
| | CDRH1: 45-54; CDRH2: 69-85; CDRH3: 116-131) |
| VL chain | gacatccagatgacacaatcttcatcctccttttctatatctctaggagacagagtcaccattacttgcagggcaagtgag |
| DNA | gacttatataatcgattagcctggtatcagcagaaaccaggaaatgctcctaggctcttaatatctggtgcaaccagtttgg |
| | aaactggggttccttcaagattcagtggcagtggatctgggaaggatttcactctcagcattcccagtgttcagactgaag |
| | atgttggtacttactactgtcaacagtatcggtatactccgtggacgttcggaggaggcaccacgctgaat (SEQ ID |
| | NO: 4; CDRL1: 70-96; CDRL2: 148-168; CDRL3: 265-291) |
| VL chain | DIQMTQSSSSFSISLGDRVTITCRASEDLYNRLAWYQQKPGNAPRLLISGATSLET |
| AA | GVPSRFSGSGSGKDFTLSIPSVQTEDVGTYYCQQYRYTPWTFGGGTTLN (SEQ ID |
| | NO: 5; CDRL1: 24-32; CDRL2: 50-56; CDRL3: 89-97) |

B7B23

| | |
|---|---|
| VH chain | atgggatggagctgtatcatgttctttctggtagcaacagctacaggtgtgcactcccaggtccagctgcagcagtctggg |
| DNA | gctgagctggtgaggcctggggtctcagtgaagatttcctgcaagggttctggctacacattcactgattatactatgcact |
| | gggtgaagcagagtcatgcaaagagtctagagtggattggagttattagtccttactatggtgatgctagctacaaccag |
| | aagttcaagggcaaggccacaatgactgtagacaaatcctccagcacagcctatatggaacttgccagactgacatct |
| | gaagattctgccatctattactgtgcaagagggttactacgtgggtttgcttactggggccaagggactctggtcactgtct |
| | ct (SEQ ID NO: 6; CDRH1: 133-162; CDRH2: 205-255; CDRH3: 346-375) |
| VH chain | MGWSCIMFFLVATATGVHSQVQLQQSGAELVRPGVSVKISCKGSGYTFTDYTM |
| AA | HWVKQSHAKSLEWIGVISPYYGDASYNQKFKGKATMTVDKSSSTAYMELARLTS |
| | EDSAIYYCARGLLRGFAYWGQGTLVTVS (SEQ ID NO: 7; CDRH1: 45-54; |
| | CDRH2: 69-85; CDRH3: 116-125) |
| VL chain | tgggatttgagggagtcacagactcaggtcttcatgtttcttatgttcagggtattgggtgcctgtgcagacattgtgatgaca |
| DNA | cagtctccatcctccaaggatatgtcagtaggacagaaggtcactatgaggtgcaagtccagtaagagccttttaaatag |
| | tagcaatcaaaagaaatatttggcctggtaccagcagaaaccaggacagtctcctaaacttctggtatactttgcatccatt |
| | agggaatctggggtccctgatcgcttcataggcagtggatctgggacagatttcactcttaccatcagcagtgtgcaggtt |
| | gaagacctggcagattacttctgtcagcaacattatagcactccgtggacgttcggtggaggc (SEQ ID NO: 8; |
| | CDRL1: 139-189; CDRL2: 235-255; CDRL3: 352-378) |
| VL chain | WDLRESQTQVFMFLMFRVLGACADIVMTQSPSSKDMSVGQKVTMRCKSSKSL |
| AA | LNSSNQKKYLAWYQQKPGQSPKLLVYFASIRESGVPDRFIGSGSGTDFTLTISSV |
| | QVEDLADYFCQQHYSTPWTFGGG (SEQ ID NO: 9; CDRL1: 47-63; CDRL2: 79- |
| | 85; CDRL3: 118-126) |

B11B4

| | |
|---|---|
| VH chain | atgggatggagctgtatcatgttcttcctagtggcaacagctataggtgtccactcccaggttcagcttcagcagtctgggg |
| DNA | gtgagctggtgaagcctggggcctcagtgaagatgtcctgcaaggcttttggctacaccttcacttcctttccaatagagtg |
| | gatgaagcagaatcatgggaagagcctagagtggattggaaattttcatccttataatgatgatactaagtacaatgaaaa |
| | attcaacggcaaggccaaattgactgcagaaacatcctctaaaacagtctatttggagctcagccgattaacatctgatg |
| | actgcagtgtttattactgtgcaagagggagtgattattcgtttggtttggactactggggtcaaggaacctcagtcaccgtc |
| | tcctca (SEQ ID NO: 10; CDRH1: 133-162; CDRH2: 205-261; CDRH3: 346-381) |
| VH chain | MGWSCIMFFLVATAIGVHSQVQLQQSGGELVKPGASVKMSCKAFGYTFTSFPIE |
| AA | WMKQNHGKSLEWIGNFHPYNDDTKYNEKFNGKAKLTAETSSKTVYLELSRLTSD |
| | DCSVYYCARGSDYSFGLDYWGQGTSVTVSS (SEQ ID NO: 11; CDRH1: 45-54; |
| | CDRH2: 69-87; CDRH3: 116-127) |
| VL chain | gtattcagggagacagacacactcatgctatgggtggtgctgctctgggttccaggttccacaggtggcattgtgctgaccc |
| DNA | aatctccagcttctttggttgtgtctctagggcagagggccaccatatcttgcagagccagtgaaagtgttgatagttatggc |
| | tatagttttatgcactggtaccagcagaaaccaggacagccacccaaactcctcatttatcgtgcatccaacctaaaatct |
| | gggatccctgccaggttcagtggcagtgggtgtaggacagacttcaccctcaccattaatcctgtggaggctgatgatgt |
| | tgcaacctattactgtcagcaaagtaatgaggatccttggacgttcggtggaggcaccaaagctggaaat (SEQ ID |
| | NO: 12; CDRL1: 136-180; CDRL2: 226-246; CDRL3: 343-369) |
| VL chain | VFRETDTLMLWVVLLWVPGSTGGIVLTQSPASLVVSLGQRATISCRASESVDSY |
| AA | GYSFMHWYQQKPGQPPKLLIYRASNLKSGIPARFSGSGCRTDFTLTINPVEADDV |
| | ATYYCQQSNEDPWTFGGGTKAGN (SEQ ID NO: 13; CDRL1: 46-60; CDRL2: 76- |
| | 82; CDRL3: 115-123) |

B19C11

| | |
|---|---|
| VH chain | atgagagtgctgattcttttgtggctgttcacagcctttcctggtatcctgtctgatgtgcagcttcaggagtcgggacctggc |
| DNA | ctggtgaaaccttctcagtctctgtccctcacctgcactgtcactggctactcaatcaccagtgattatgcctggaactggat |
| | ccggcagtttccaggaaacaaactggagtggatgggctacataagcaacaatggtcgcgctaggtataatccatctctca |
| | aaagtcgaatctctatcactcgagacacattcaagaaccagttcttcctgcagttgaattctgtgactactgaggacaca |
| | gccacatattactgtgcaagagaggcctcgcatgatggttccttctggtacttcgatgtctggggcgcagggaccacggtc |
| | accgtctct (SEQ ID NO: 14; CDRH1: 130-162; CDRH2: 205-252; CDRH3: 343-387) |
| VH chain | MRVLILLWLFTAFPGILSDVQLQESGPGLVKPSQSLSLTCTVTGYSITSDYAWN |
| AA | WIRQFPGNKLEWMGYISNNGRARYNPSLKSRISITRDTFKNQFFLQLNSVTTEDT |
| | ATYYCAREASHDGSFWYFDVWGAGTTVTVS (SEQ ID NO: 15; CDRH1: 44-54; |
| | CDRH2: 69-84; CDRH3: 115-129) |
| VL chain | gacagtgtgttgacccaatctccagcttgtttggttgtgtgtatagggcagagggccaccatatcttgcagagccagtgaa |
| DNA | agtgttgatagttatggctatagttttatgcattggtaccagcagaaaccaggacagccacccaaactcctcatttatcgtg |
| | catccaacctagaatgtgggatccctgccaggttcagtggcagggggtctaggacggacttcaccctcaccattactcct |
| | gtggagggtgatgatgttgcaacctattactgtcagcaaagtaatgaggatcctcggacgttcggtggaggcaccaagct |
| | ggaaatcaaa (SEQ ID NO: 16; CDRL1: 70-114; CDRL2: 160-180; CDRL3: 277-303) |
| VL chain | DSVLTQSPACLVVCIGQRATISCRASESVDSYGYSFMHWYQQKPGQPPKLLIYRA |
| AA | SNLECGIPARFSGRGSRTDFTLTITPVEGDDVATYYCQQSNEDPRTFGGGTKLEIK |
| | (SEQ ID NO: 17; CDRL1: 24-38; CDRL2: 54-60; CDRL3: 93-101) |

B33C12

| | |
|---|---|
| VH chain | atgggatggagctgtatcatgttcttcctagtggcaacagctataggtgtccactcccaggttcagcttcagcagtctgggg |
| DNA | ctgagctggtgaagcctggggcctcagtgaagatgtcctgcaaggcttttggctacaccttcacttcctttccaatagagtg |
| | gatgaagcagaatcatgggaagagcctagagtggattggaaattttcatccttctaatgatgatactaagtacaatgaaaa |
| | attcaacggcaaggccaaattgactgcagaaacatcctctaaaacagtctatttggagctcagccgattaacatctgatg |
| | actctgctgtttattactgtgcaagagggagtgattattcctttgctttggactactggggtcaaggaacctcagtcaccgtctc |
| | ctca (SEQ ID NO: 18; CDRH1: 133-162; CDRH2: 205-261; CDRH1: 346-381) |
| VH chain | MGWSCIMFFLVATAIGVHSQVQLQQSGAELVKPGASVKMSCKAFGYTFTSFPIE |
| AA | WMKQNHGKSLEWIGNFHPSNDDTKYNEKFNGKAKLTAETSSKTVYLELSRLTSD |
| | DSAVYYCARGSDYSFALDYWGQGTSVTVSS (SEQ ID NO: 19; CDRH1: 45-54; |
| | CDRH2: 69-87; CDRH3: 116-127) |
| VL chain | atggaactagtcgacatggttcttatgttgctgctgctatgggttccaggttccacaggtggcattgtgctgacccaatctcca |
| DNA | gcttctttggctgtgtctctagggcagagggccaccatatcctgcagagccagtgaaagtgttgatagttatggctatagtttt |
| | atgcactggtaccagcagaaaccaggacagccacccaaactcctcatctatcgtgcatccaacctaaaatctgggatcc |
| | ctgccaggttcagtggcagtgggtctaggacagacttcaccctcaccattaatcctgtggaggctgatgatgttgcaacc |
| | tattactgtcagcaaagtaatgaggatccttggacgttcggtggaggcaccaagctggaaatcaaa (SEQ ID |
| | NO: 20; CDRL1: 130-174; CDRL2: 220-240; CDRL3: 337-363) |
| VL chain | MELVDMVLMLLLLWVPGSTGGIVLTQSPASLAVSLGQRATISCRASESVDSYGY |
| AA | SFMHWYQQKPGQPPKLLIYRASNLKSGIPARFSGSGSRTDFTLTINPVEADDVAT |
| | YYCQQSNEDPWTFGGGTKLEIK (SEQ ID NO: 21; CDRL1: 44-58; CDRL2: 74-80; |
| | CDRL3: 113-121) |

E12B11

| | |
|---|---|
| VH chain | atgggttgggtgtggaccttgctattcctgatggcagctgcccaaagtatccaagcacagatccagttggtgcagtctgga |
| DNA | cctgagctgaagaagcctggagagacagtcaagatctcctgcaaggcttctggatataccttcacaaactatggaatga |
| | actgggtgaagcaggctccaggaaagggtttaaagtggatgggctggataaacaccaacactggagagacaacatatg |
| | ctgaagaggtcaagggacggtttgccttctctttggaaacctctgccagcactgcctatttgcagatcaacaacctcaaaa |
| | atgaggacacggctacatatttctgtgcaagatggggcccatacggtagtagcctttatttttctatggactactggggtcaa |
| | ggaacctcagtcaccgtctcctca (SEQ ID NO: 22; CDRH1: 133-162; CDRH2: 205-255; |
| | CDRH3: 346-393) |
| VH chain | MGWVWTLLFLMAAAQSIQAQIQLVQSGPELKKPGETVKISCKASGYTFTNYG |
| AA | MNWVKQAPGKGLKWMGWINTNTGETTYAEEVKGRFAFSLETSASTAYLQINNL |
| | KNEDTATYFCARWGPYGSSLYFSMDYWGQGTSVTVSS (SEQ ID NO: 23; |
| | CDRH1: 45-54; CDRH2: 69-85; CDRH3: 116-131) |
| VL chain | gatatccagatgacacaatcttcatcctccttttctgtatctctaggagacagactcaccattacttgcaaggcaagtgagg |
| DNA | acatatataatcggttagcctggtatcaacagaaaccaggaaatgctcctaggctcttaatatatggtgcaaccagtttgg |
| | aaagtggggttccttcaagattcagtggcagtggatctggaaaggattacactctcagcattcccagttttcagagagaa |
| | gatggtggtagcaacttatgtcaacagtatcggaatagagcgtggacgttcggaggagggaccaagctggaaataaa |
| | acgg (SEQ ID NO: 24; CDRL1: 70-102; CDRL2: 148-168; CDRL3: 265-291) |
| VL chain | DIQMTQSSSSFSVSLGDRLTITCKASEDIYNRLAWYQQKPGNAPRLLIYGATSLES |
| AA | GVPSRFSGSGSGKDYTLSIPSFQREDGGSNLCQQYRNRAWTFGGGTKLEIKR |
| | (SEQ ID NO: 25; CDRL1: 24-34; CDRL2: 50-56; CDRL3: 89-97) |

F8B30

| | |
|---|---|
| VH chain | atggcttgggtgtggaccttgctattcctgatggcagctgcccaaagtatccaagcacagatccagttggtgcagtctgga |
| DNA | cctgagctgaagaagcctggagagacagtcaagatctcctgcaaggcttctggttataccttcacagactattcaatgca |
| | ctgggtgaagcaggctccaggaaagggtttaaagtggatgggctggataaacactgagactggtgggccaacatatgc |
| | cgatgacttcaagggacggtttgccttctctttggaaacctctgccaccactgcctatttgcagatcaacaacctcaaaaat |
| | gaggacacggctacatatttctgtagtagagatgtcgacctctactttgactactggggccaaggcaccactctcacagtc |
| | tct (SEQ ID NO: 26; CDRH1: 133-162; CDRH2: 205-255; CDRH3: 346-375) |
| VH chain | MAWVWTLLFLMAAAQSIQAQIQLVQSGPELKKPGETVKISCKASGYTFTDYSM |
| AA | HWVKQAPGKGLKWMGWINTETGGPTYADDFKGRFAFSLETSATTAYLQINNLK |
| | NEDTATYFCSRDVDLYFDYWGQGTTLTVS (SEQ ID NO: 27; CDRH1: 45-54; |
| | CDRH2: 69-85; CDRH3: 116-125) |
| VL chain | gacatccagatgactcagtctccagcctccctatctgcatctgtgggagaaactgtcaccatcacatgtcgaacaagtga |
| DNA | gaatatttacagttatttagcatggtatcagcagaaacagggaaaatctcctcagctcctggtctataatgcaaaaaccctg |
| | gtagaaggtgtgccatcgaggttcagtggcagtggatcaggcacacagttttctgtgaagatcaacagcctgcagcctg |
| | aagattttgggaattattactgtcaacatcattatgggattccgttcacgttcggaggagggaccaaactagaaataaaa |
| | (SEQ ID NO: 28; CDRL1: 70-102; CDRL2: 148-168; CDRL3: 265-291) |
| VL chain | DIQMTQSPASLSASVGETVTITCRTSENIYSYLAWYQQKQGKSPQLLVYNAKTLV |
| AA | EGVPSRFSGSGSGTQFSVKINSLQPEDFGNYYCQHHYGIPFTFGGGTKLEIK (SEQ |
| | ID NO: 29; CDRL1: 24-34; CDRL2: 50-56; CDRL3: 89-97) |

G11B22

| | |
|---|---|
| VH chain | atgggttgggtgtggaccttgctattcctgatggcagctgcccaaagtatccaagcacagatccagttggtgcagtctgga |
| DNA | cctgaggtgaagaagcctggagagacagtcaagatctcctgcaaggcttctgggtataccttcacaaagtatggaatga |
| | actgggtgaagcaggctccaggaaagggtttaaagtggatgggctggataaacaccaacactggagagccaacatatg |
| | ctgaagagttcaagggacggtttgccttctctttggaaacctctgccagcactgcctttttgcagatcaacaacctcaaaa |
| | atgaggacacggctgcatatttctgtgcaagatggggcccatacggtagtagcctttactatgctatggactactggggtca |
| | aggaacctcagtcaccgtctct (SEQ ID NO: 30; CDRH1: 133-162; CDRH2: 205-255; |
| | CDRH3: 346-393) |
| VH chain | MGWVWTLLFLMAAAQSIQAQIQLVQSGPEVKKPGETVKISCKASGYTFTKYG |
| AA | MNWVKQAPGKGLKWMGWINTNTGEPTYAEEFKGRFAFSLETSASTAFLQINNL |
| | KNEDTAAYFCARWGPYGSSLYYAMDYWGQGTSVTVS (SEQ ID NO: 31; |
| | CDRH1: 45-54; CDRH2: 69-85; CDRH3: 116-131) |
| VL chain | gacatccagatgacacaatcttcatcctccttttctgtatctgtaggagacagagtcaccattacttgcagggcaagtgag |
| DNA | gacatatataatcggttagcctggtatcagcagaaaccaggaaatgctcctaggctcttaatatctggtgcaaccagtttg |
| | gaaactggggttccttcaagattcagtggcagtggatctgggaaggattacactctcagcattcccagtgttcagagaga |
| | agatggaggtagcaacttatgtcagccatcacggagtagaccgtgcacgttcggaggaggcaccaagctgaaatcaa |
| | agcga (SEQ ID NO: 32; CDRL1: 70-102; CDRL2: 148-168; CDRL3: 265-291) |
| VL chain | DIQMTQSSSSFSVSVGDRVTITCRASEDIYNRLAWYQQKPGNAPRLLISGATSLET |
| AA | GVPSRFSGSGSGKDYTLSIPSVQREDGGSNLCQPSRSRPCTFGGGTKLKSKR (SEQ |
| | ID NO: 33; CDRL1: 24-34; CDRL2: 50-56; CDRL3: 89-97) |

TABLE 2

| Clone | K_{assoc} (Ms)⁻¹ | K_{dissoc} (s⁻¹) | KD (nM) |
|---|---|---|---|
| B2A18, (IgG1, κ) | 1.68 × 10⁶ ± 8.80 × 10³ | 1.75 × 10⁻⁴ ± 5.09 × 10⁻⁶ | 0.104 ± 0.00307 |
| B7B23 (IgG1, κ) | 1.69 × 10⁶ ± 8.22 × 10³ | 6.96 × 10⁻⁵ ± 4.64 × 10⁻⁶ | 0.0413 ± 0.00276 |
| B11B4 (IgG2b, κ) | 8.25 × 10⁵ ± 7.51 × 10³ | 2.02 × 10⁻⁴ ± 8.60 × 10⁻⁶ | 0.245 ± 0.0107 |
| B19C11 (IgG1, κ) | 6.22 × 10⁵ ± 7.55 × 10³ | 4.93 × 10⁻⁴ ± 1.10 × 10⁻⁵ | 0.793 ± 0.0202 |
| B33C12 (IgG2a, κ) | 8.35 × 10⁵ ± 8.43 × 10³ | 2.99 × 10⁻⁴ ± 9.69 × 10⁻⁶ | 0.359 ± 0.0122 |
| E12B11 (IgG1, κ) | 1.20 × 10⁶ ± 8.69 × 10³ | 1.72 × 10⁻⁴ ± 6.94 × 10⁻⁶ | 0.143 ± 0.00585 |
| F8B30 (IgG1, κ) | 6.15 × 10⁵ ± 8.48 × 10³ | 4.44 × 10⁻⁴ ± 1.26 × 10⁻⁵ | 0.723 ± 0.0228 |
| G11B22 (IgG1, κ) | 1.37 × 10⁶ ± 1.08 × 10⁴ | 5.79 × 10⁻⁴ ± 7.54 × 10⁻⁶ | 0.423 ± 0.00644 |

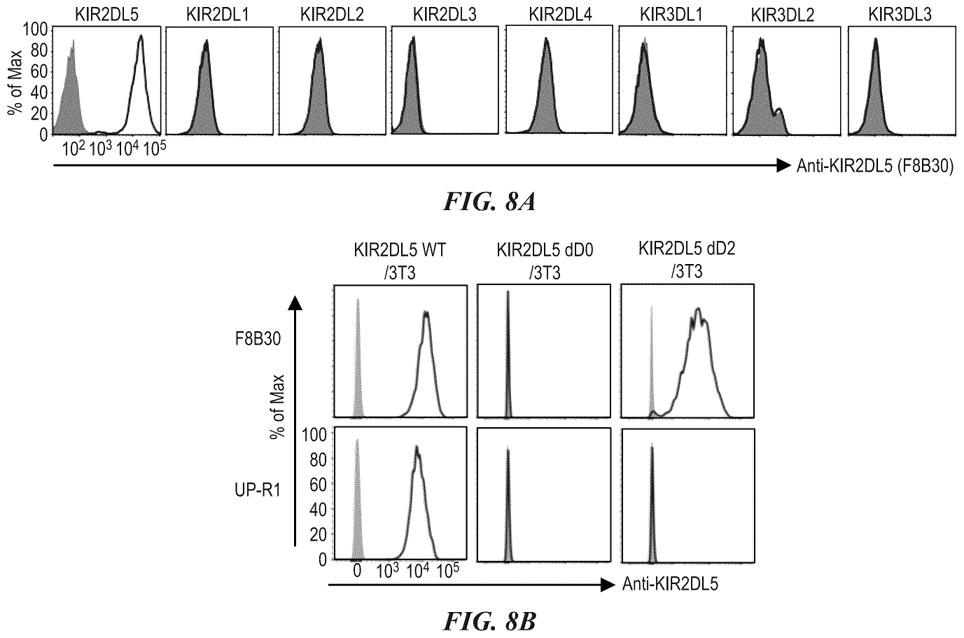

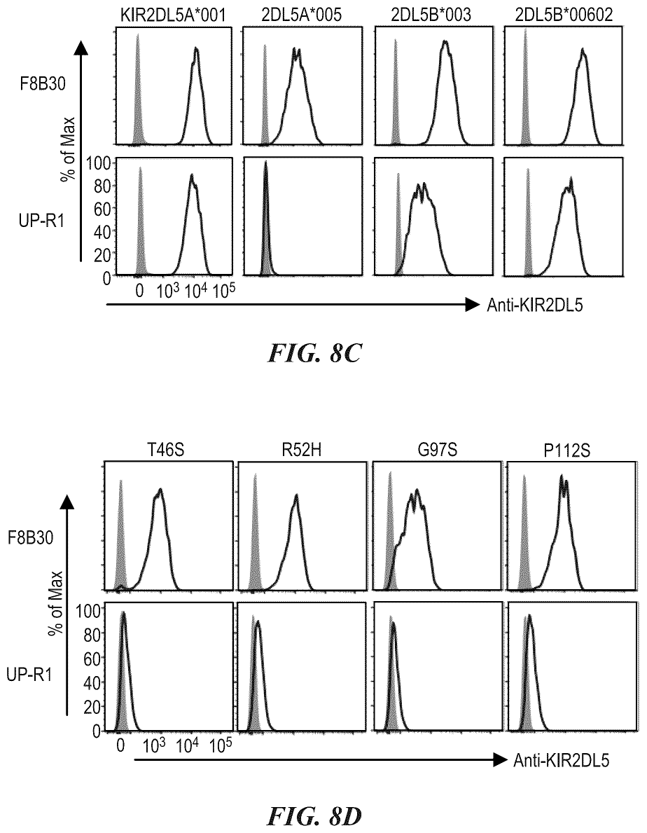

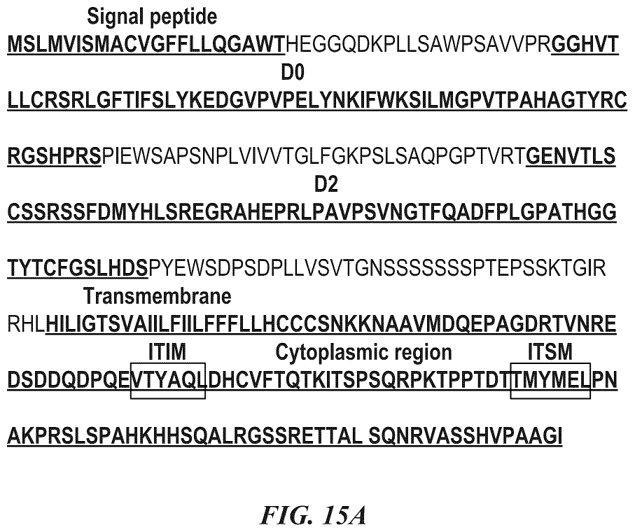

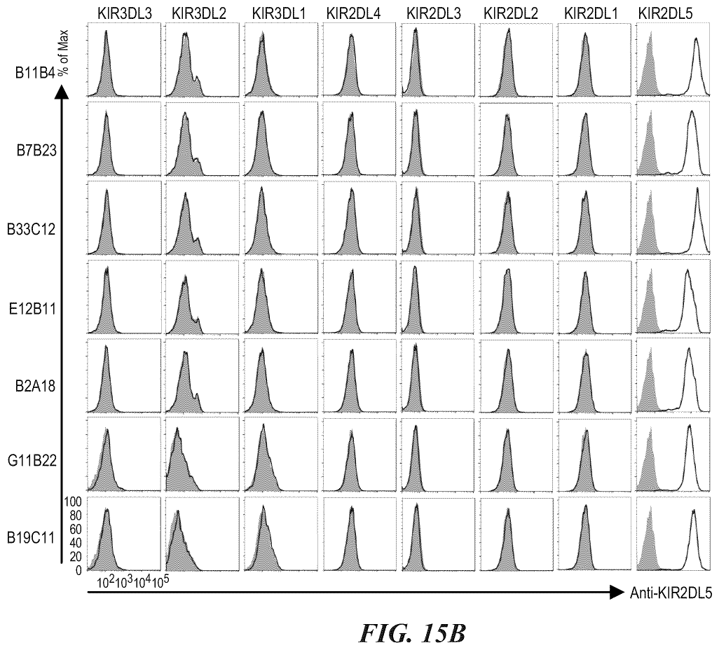

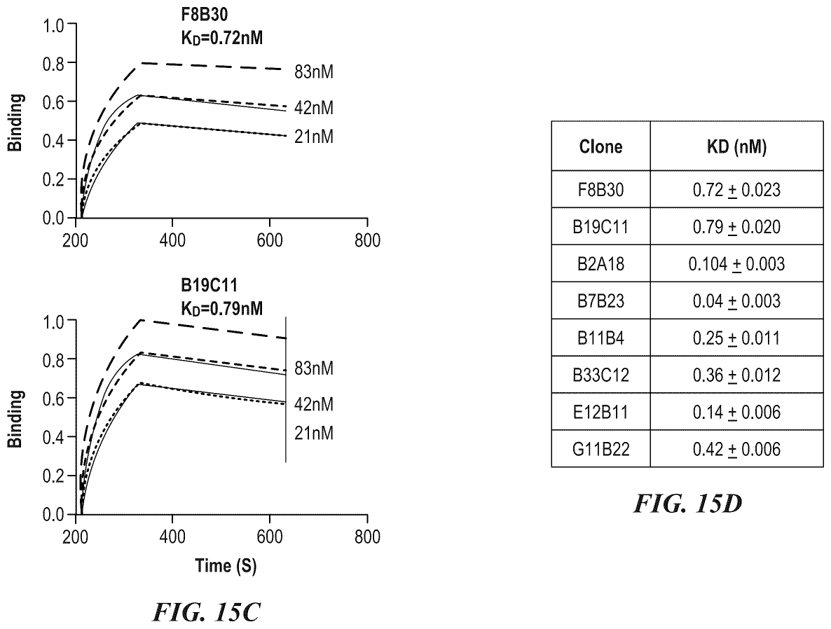

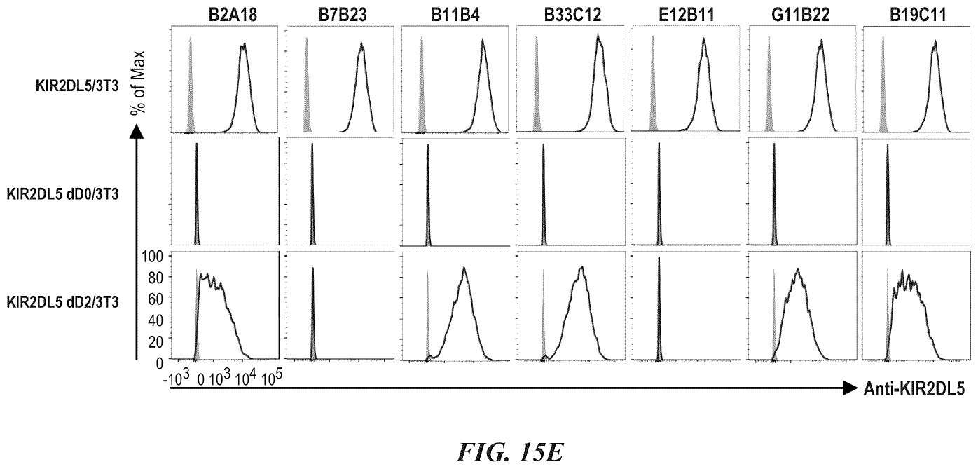

As shown in FIGS. 8A and 15B, 8 anti-KIR2DL5 specific mAbs, which had no cross-reaction with other KIRs, were generated. Clone F8B30 displayed high affinity against KIR2DL5 (K_{D}=0.72 nM) as determined by biolayer interferometry (FIGS. 15C and 15D). Two truncated KIR2DL5 proteins were expressed by removing the DO or D2 domain to determine the KIR2DL5 recognition pattern of mAbs of the present technology. As shown in FIGS. 8B and 15E, in comparison with UP-R1, which required both DO and D2 domains for KIR2DL5 recognition, several of the currently disclosed anti-KIR2DL5 mAbs, including F8B30, bound to KIR2DL5 through the DO domain.

Notably, FIG. 8C shows that F8B30, but not UP-R1, efficiently recognized cell surface-expressed 2DL5A*005. As shown by FIGS. 8C and 16A, 2DL5B*003 and 2DL5B*00602 are also bound by F8B30 and other clones.

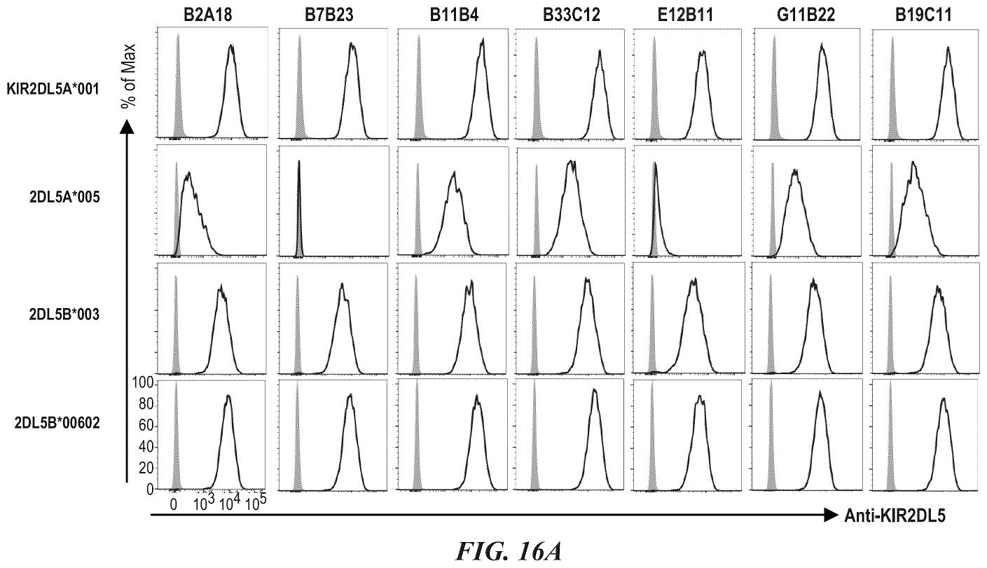

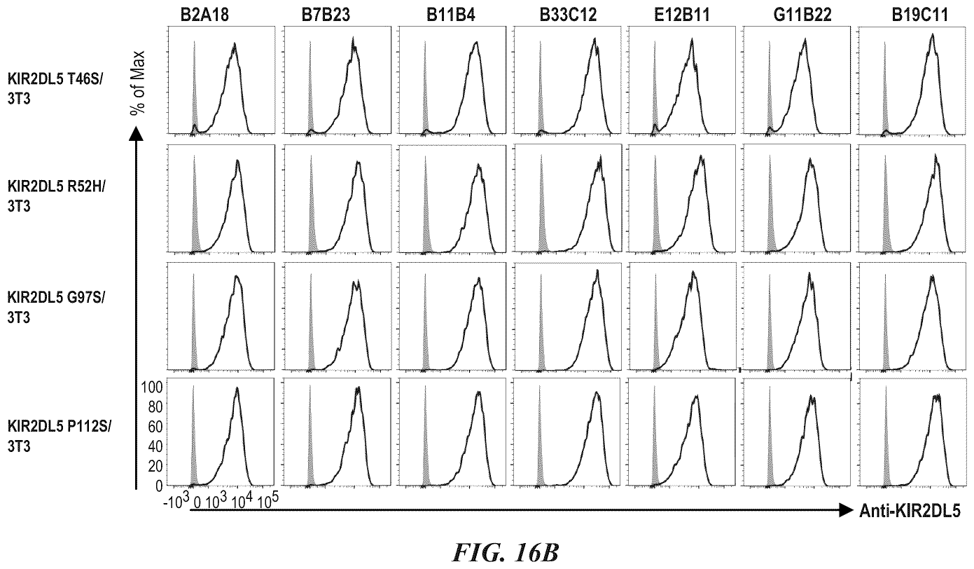

The four DO domain polymorphism variants, namely, T46S, R52H, G97S, and P112S, were generated by mutating KIR2DL5A*001. As shown in FIGS. 8D and 16B, all variants were recognized by mAbs of the present technology, including F8B30 with a much lower half maximal effective concentration (EC₅₀; ranging from 8.6 to 43.6 nM) than that of UP-R1 (ranging from 391.1 to 875.9 nM) (Table 3). Collectively, the results showed that mAbs of the present technology against the DO domain of KIR2DL5 efficiently recognized different KIR2DL5 alleles.

TABLE 3
Comparison of the EC₅₀ of F8B30 and
UP-R1 binding to the indicated
KIR2DL5 D0 domain variants

| | F8B30 | UP-R1 |
|---|---|---|
| D0 | EC50 | EC50 |
| variants | (nM) | (nM) |
| T46S | 8.6 | 610.0 |
| R52H | 9.2 | 442.4 |
| G97S | 43.6 | 875.9 |
| P112S | 9.4 | 391.1 |

### Example 3: KIR2DL5 Expression

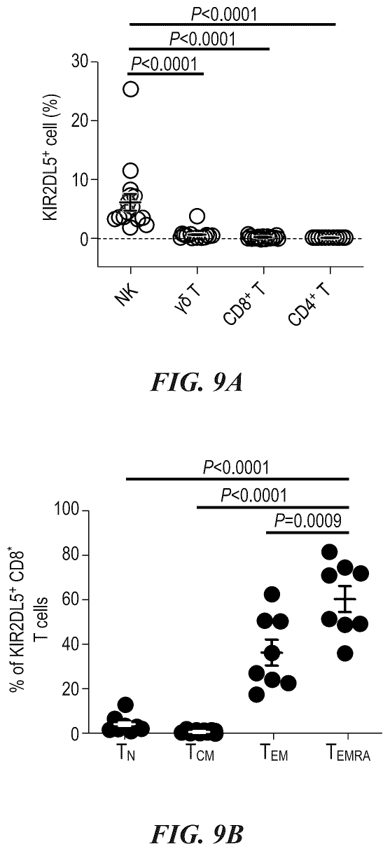

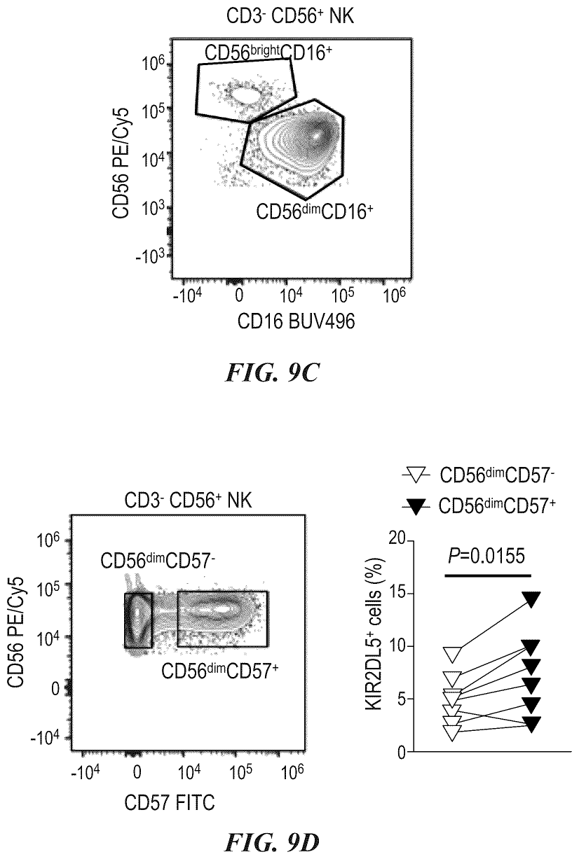

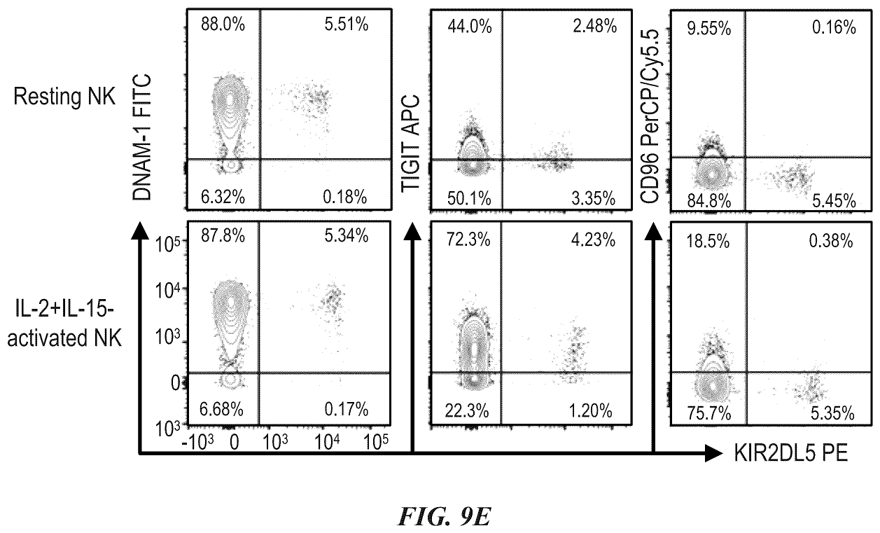

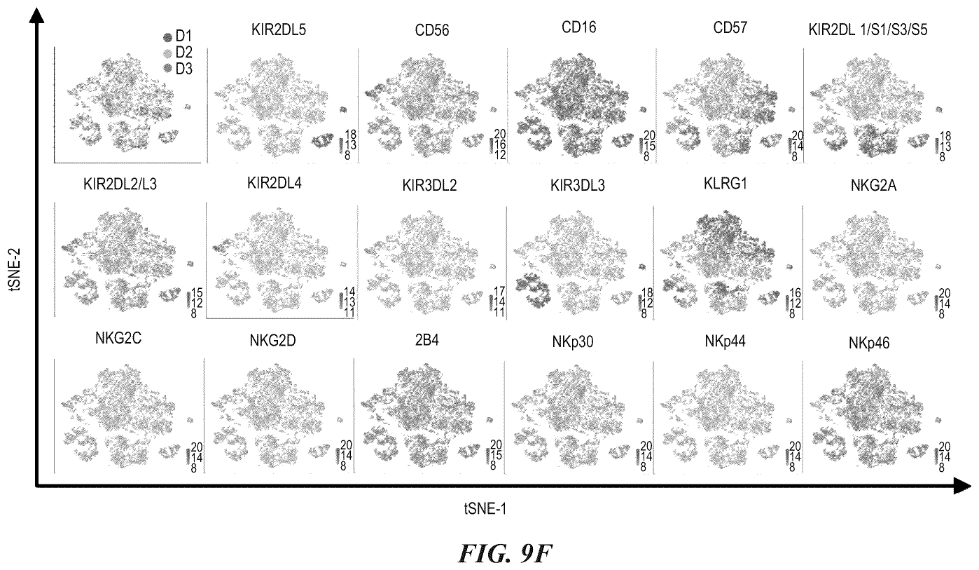

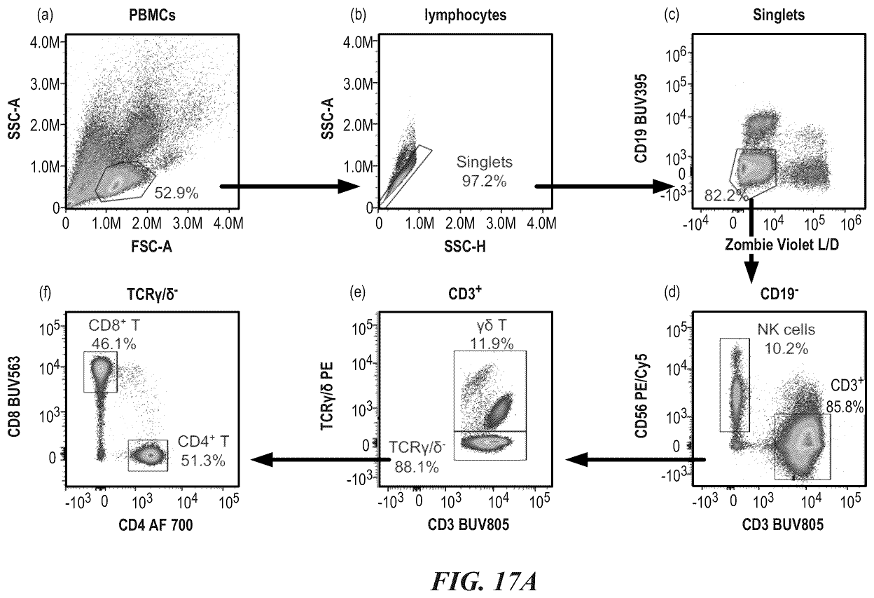

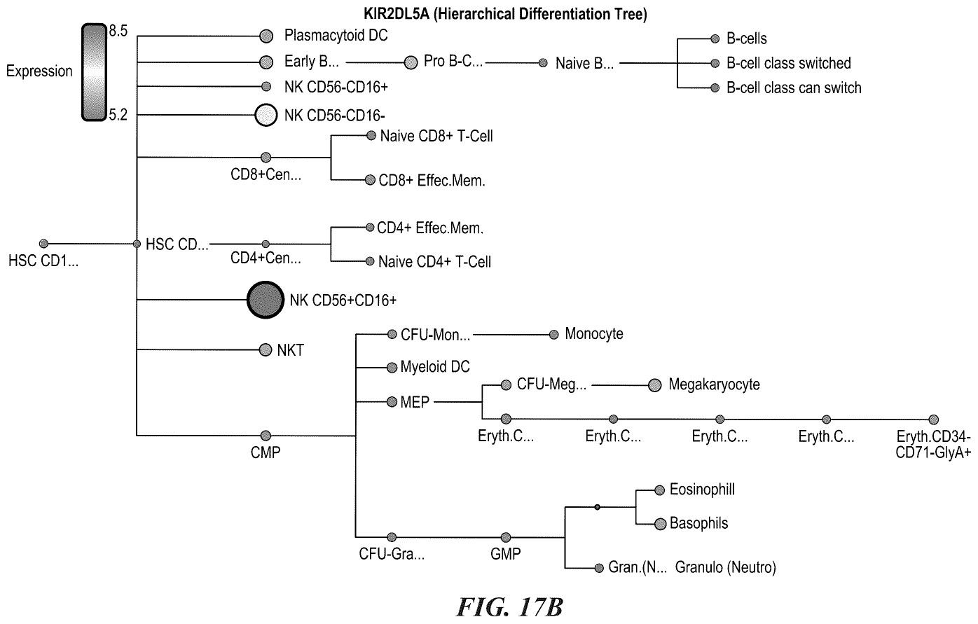

Based on the performance of the mAbs of the present technology over UP-R1 for KIR2DL5 recognition (see e.g., Example 2), F8B30 was used to redefine the KIR2DL5 expression pattern in human immune cells. KIR2DL5 protein was expressed on both innate (NK and γδ T cells) and adaptive (CD8⁺ T cells) immune cells from human peripheral blood (FIGS. 9A and 17A). Additionally, KIR2DL5⁺ CD8⁺ T cells were mainly distributed in terminally differentiated (Temra) and, to a lesser extent, effector memory cell subsets, whereas KIR2DL5 expression was very low or undetectable in naive (Tn) and central memory (Tcm) CD8⁺ T cells (FIG. 9B).

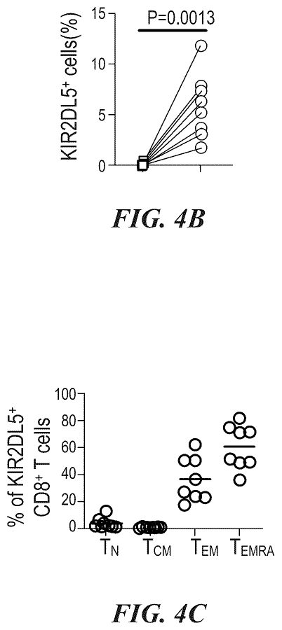

By FACS analysis of PBMC with the anti-KIR2DL5 mAbs of Example 2, it was found that KIR2DL5 is widely expressed on the cell surface of innate immune cells (NK cells, γδT cells) and adaptive immune cells (CD8 Tcells, CD4 T cells) (FIG. 4A). KIR2DL5 is mainly expressed on CD56^{dim}CD16+ NK cells (FIG. 4B) and terminally differentiated effector memory CD8 T cells (T_{EMRA}) (FIG. 4C).

According to the mRNA expression pattern (FIG. 17B), KIR2DL5 protein was predominantly expressed on NK cells, particularly on the CD56^{dim}CD16⁺ NK subset (FIG. 9C), which is more differentiated and cytolytic than the CD56^{bright}CD16⁻ subset (Moretta 2010). CD57 defines a functionally distinct NK cell population that is highly mature and terminally differentiated (Lopez-Verges 2010). A higher proportion of CD56^{dim}CD57⁺ cells expressed KIR2DL5, as compared with the CD56^{dim}CD57⁻ NK subset (FIG. 9D). Stimulatory cytokines, such as IL-2, IL-12, IL-15, and IL-18, drive NK cell activation and maturation (Wu 2017). TIGIT, DNAM-1, and CD96 are well-established receptors for PVR. TIGIT and CD96, but not KIR2DL5, were upregulated in response to exogenous stimulation with IL-2 and IL-15 (FIG. 9E). Moreover, KIR2DL5 was coexpressed with DNAM-1 and TIGIT, whereas its expression was mutually exclusive from CD96 expression on both resting and activated NK cells (FIG. 9E). Lastly, analysis of NK cell receptors by high-dimensional flow cytometry revealed that KIR2DL5 was clonally distributed in CD56^{dim}CD16⁺ NK cells and was coordinately expressed with the other NK cell receptors and KIRs (FIG. 9F).

### Example 4: Effect of KIR2DL5 on NK Cell Function

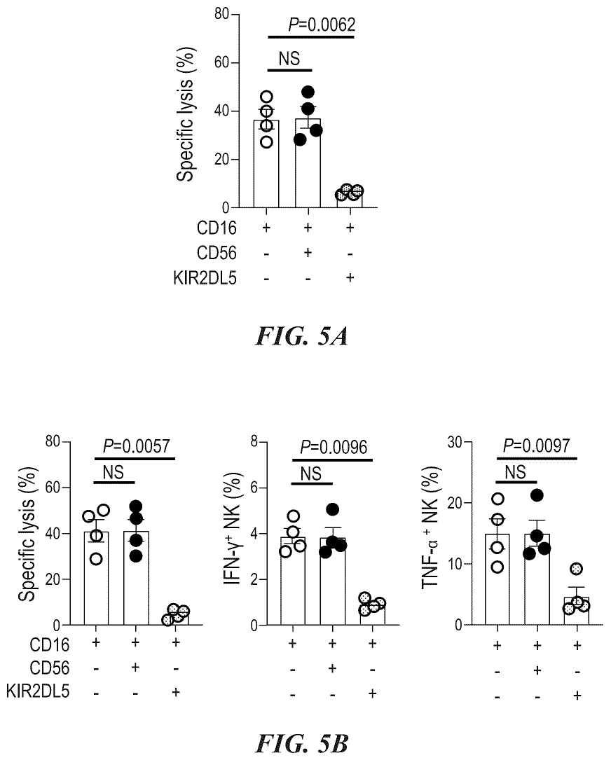

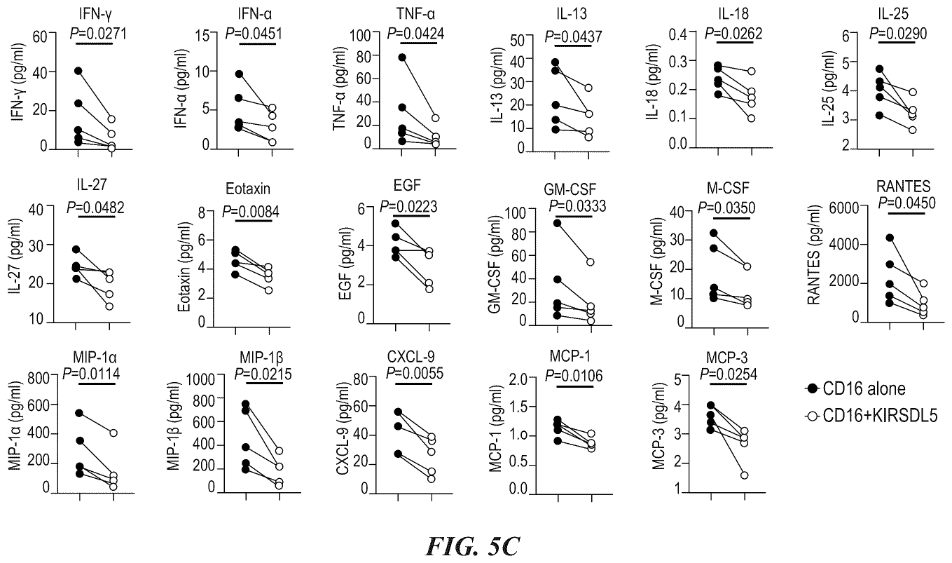

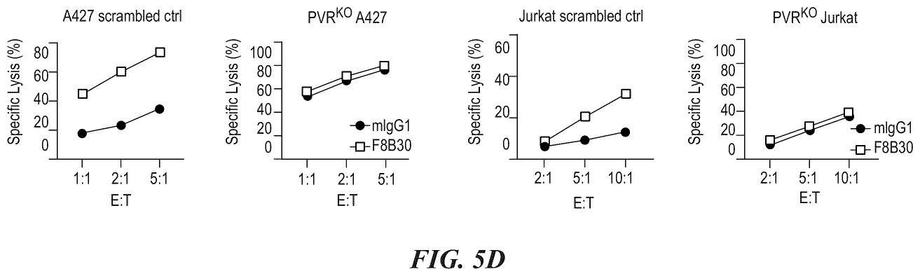

Primary KIR2DL5+ NK cells were sorted out and an NK cell-based redirected cytotoxicity assay was performed as reported previously (Wei 2021). Co-engagement of CD16 with KIR2DL5, but not with CD56, significantly inhibited lysis of P815 (FIG. 5A) and expression of CD107a, IFN-γ and TNF-α of NK cells (FIG. 5B). Consistently, KIR2DL5 markedly decreased other cytokine and chemokine production of NK cells (FIG. 5C), including IL-13, IL-18, IL-25, IL-27, Eotaxin, EGF, GM-CSF, M-CSF, RANTES, MIP-1α, MIP-1β, CXCL9, MCP-1, and MCP3.

To explore the effect of KIR2DL5-PVR engagement on NK-mediated tumor cell lysis, human tumor lines A427 (solid tumor) and Jurkat (hematologic malignancy) expressing endogenous PVR were treated with CRISPR-Cas9 to knock-out PVR (PVR^{ko} A427, PVR^{ko} Jurkat) or scramble negative control. These cells were used as targets and co-cultured with primary KIR2DL5+ NK cells and anti-KIR2DL5 blocking mAb F8B30 or control mIgG1 (FIG. 5D). Anti-KIR2DL5 blocking mAb F8B30 significantly enhanced the lysis of scramble negative control A427 and Jurkat, but this effect was lost when PVR was knocked out in A427 and Jurkat. These results demonstrate that KIR2DL5 inhibits NK cell function and that engagement of PVR on tumor cells and KIR2DL5 on immune cells mediates tumor immune evasion.

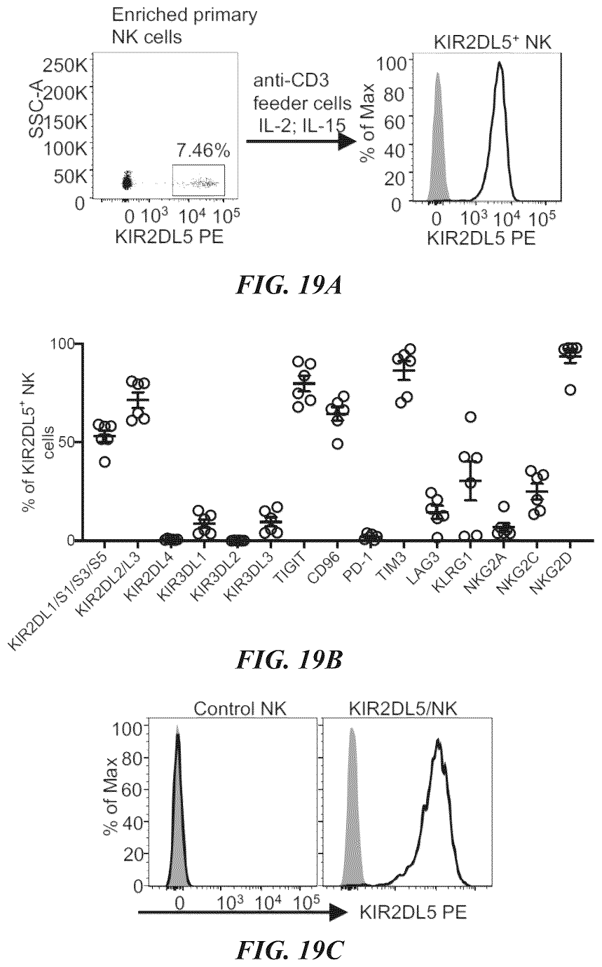

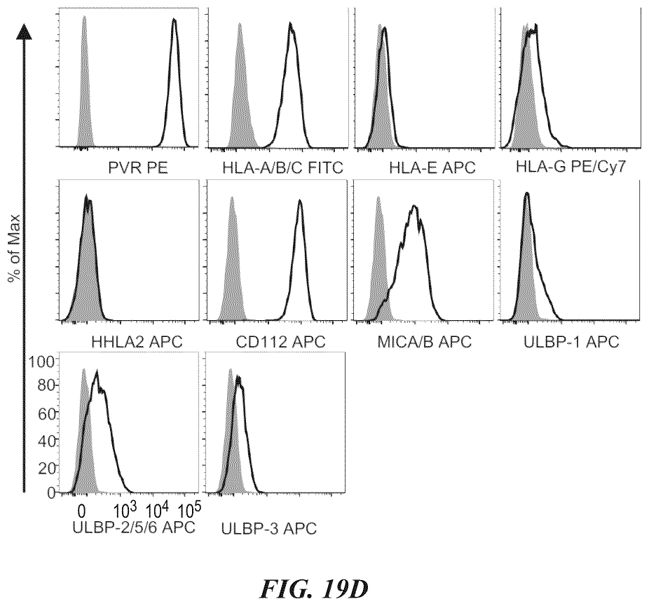

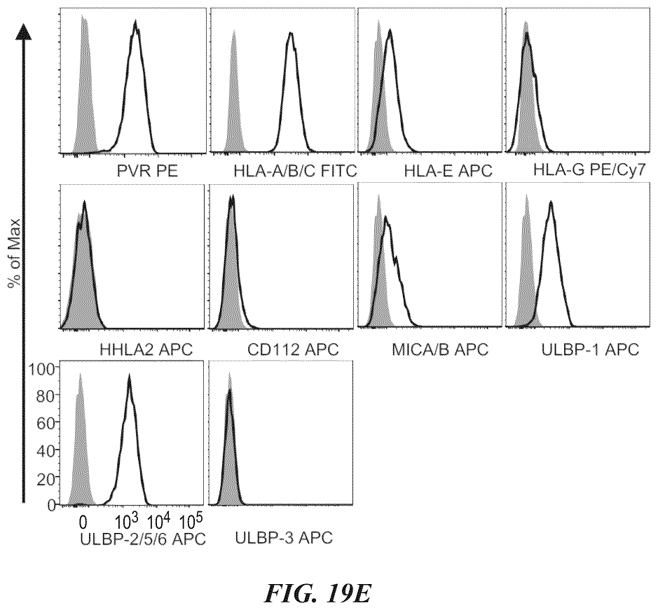

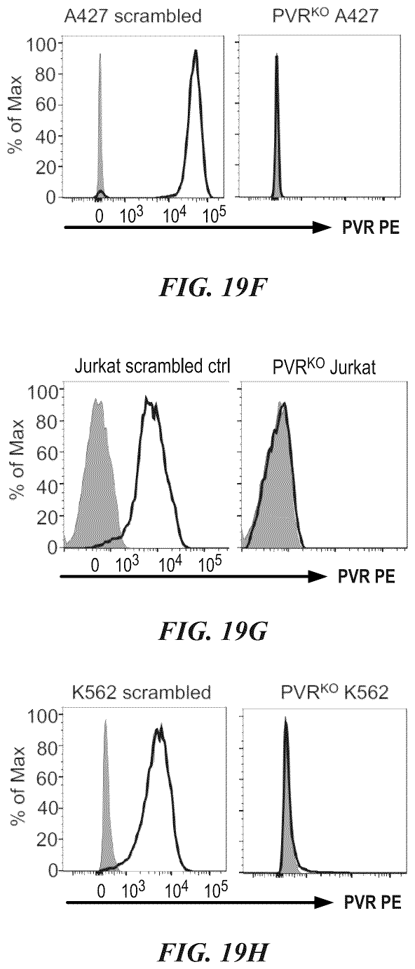

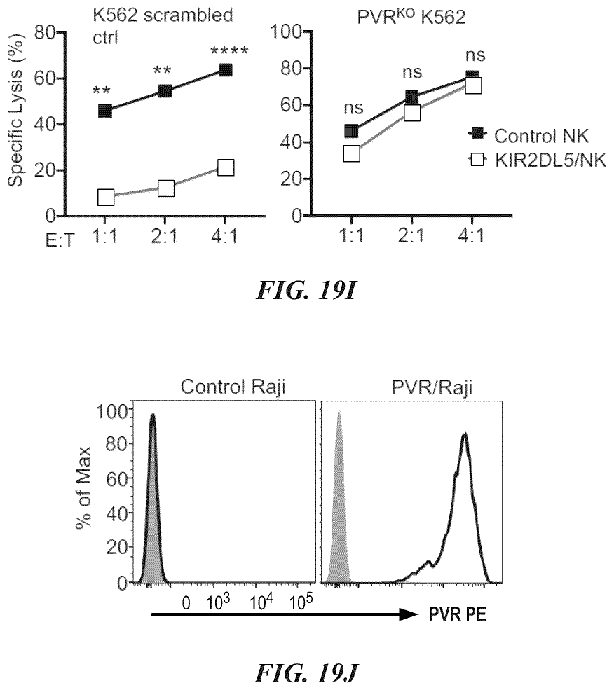

To validate whether KIR2DL5 directly inhibits primary NK cell functions, KIR2DL5⁺ NK cells from human PBMCs was sorted out, which confirmed stable KIR2DL5 expression after activation and expansion (FIG. 19A). High expression of other immune inhibitory receptors, including KIR2DL1/L2/L3, TIGIT, CD96, and TIM3, and the immune stimulatory receptor NKG2D, was also detected on those expanded KIR2DL5⁺ NK cells (FIG. 19B). An NK cell-based, CD16-induced redirected cytotoxicity assay showed that the co-engagement of CD16 with KIR2DL5, but not with CD56, significantly inhibited target cell P815 killing and NK cell degranulation (CD107a) as well as IFN-γ and TNF-α production (FIGS. 5A and 5B). By performing a 65-plex human cytokine/chemokine array experiment, it was observed that KIR2DL5 markedly decreased the production of a broad spectrum of cytokines/chemokines, including IL-13, IL-18, IL-25, IL-27, eotaxin, EGF, GM-CSF, M-CSF, RANTES, MIP-1α, MIP-1β, CXCL-9, and others (FIG. 5C).

The effect of the KIR2DL5-PVR engagement on NK-mediated tumor cell lysis was examined. Primary NK cells with KIR2DL5 (FIG. 19C) were transduced and cocultured with human lung cancer A427 and leukemic Jurkat tumor cells that expressed endogenous PVR. A427 and Jurkat cells displayed a distinct expression profile of ligands for NK cell receptors (FIGS. 19D and 19E) and were susceptible to NK cell killing. While the presence of KIR2DL5 dramatically suppressed NK cytolytic activity against PVR⁺ tumor cells (scrambled control), this effect was eliminated upon the deletion of PVR in tumor cells by CRISPR/Cas9 (PVR^{KO}) (FIGS. 5D, 19F, and 19G). A similar observation was obtained with another leukemic K562 tumor cells (FIGS. 19H and 19I).

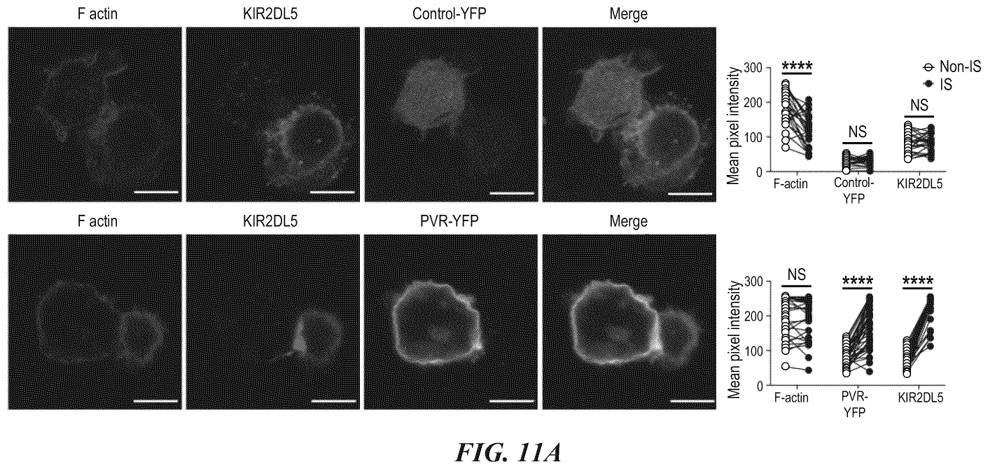

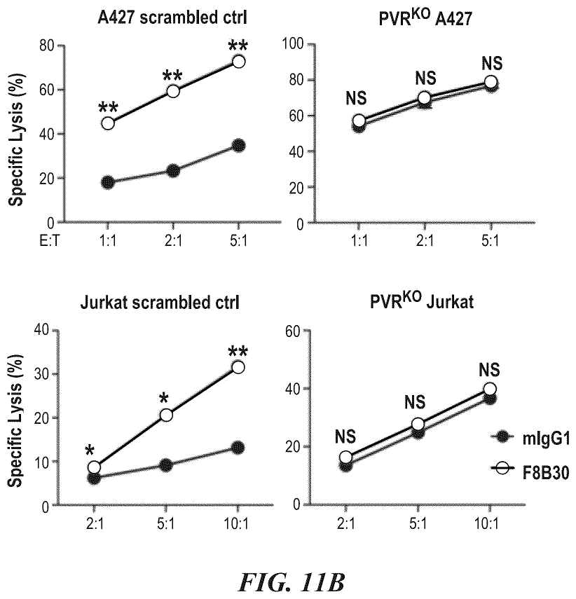

To investigate whether KIR2DL5-PVR interaction mediated inhibitory synapse formation, primary KIR2DL5⁺ NK cells was incubated with Raji cells expressing PVR-YFP (PVR/Raji) or control-YFP (Control Raji) fusion protein (FIG. 19J). In the absence of PVR on the target cells, it was observed that KIR2DL5 distributed evenly on the NK cell surface while F-actin accumulated at the interface, indicating the formation of a lytic synapse (FIG. 11A, top). By contrast, in the presence of PVR on the target cells, KIR2DL5 clustering with PVR, but no F-actin polarization, was observed at the NK-Raji interface (FIG. 11A, bottom), indicating the impairment of actin reorganization and the formation of an inhibitory synapse.

The impact of direct blockade of KIR2DL5 on NK cell functions against PVR⁺ human tumors was examined. As shown in FIG. 11B (scrambled control), the currently disclosed anti-KIR2DL5 mAb F8B30, which was able to effectively block KIR2DL5-PVR interaction, significantly enhanced the tumor lysis by KIR2DL5⁺ primary NK cells. The effect of F8B30 was also dependent on PVR, as this mAb lost the enhanced effect on NK functions in the absence of PVR (FIG. 11B, PVR^{KO}) Example 5: Therapeutic efficacy of KIR2DL5 blockade

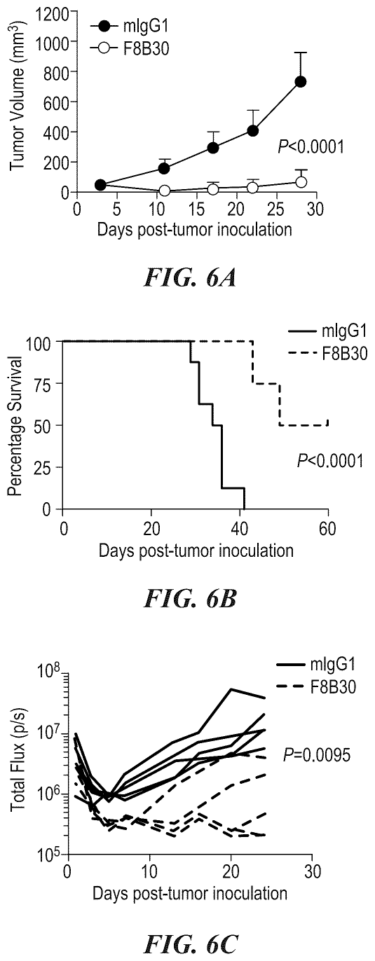

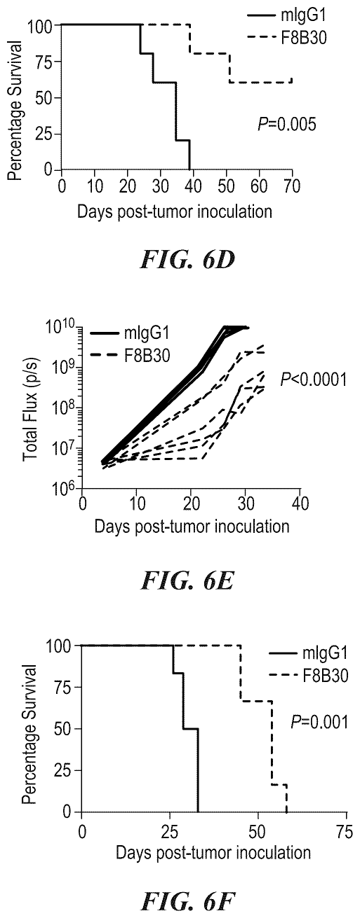

Therapeutic efficacy of KIR2DL5 blockade was evaluated with primary NK cells in vivo in three humanized mouse models. NSG mice were subcutaneously engrafted with A427 and then treated with expanded primary KIR2DL5+ NK cells intratumorally, as well as anti-KIR2DL5 blocking mAb F8B30 or mIgG1. F8B30 significantly reduced tumor growth (FIG. 6A) and increased mice survival (FIG. 6B) when compared with mIgG1.

Using a more physiologically relevant human lung cancer model, mice were inoculated with A427 cells intravenously and then reconstituted with primary KIR2DL5+ NK cells and treated with F8B30 or mIgG1. Tumor growth in the lung was significantly inhibited (FIG. 6C) and survival was significantly prolonged (FIG. 6D).

Next, efficacy was evaluated in the hematologic malignancy Jurkat xenograft tumor model. NSG mice were inoculated with Jurkat cells intravenously and then reconstituted with primary KIR2DL5+ NK cells and treated with F8B30 or mIgG1. Tumor growth in vivo was significantly inhibited (FIG. 6E) and survival was significantly prolonged (FIG. 6F).

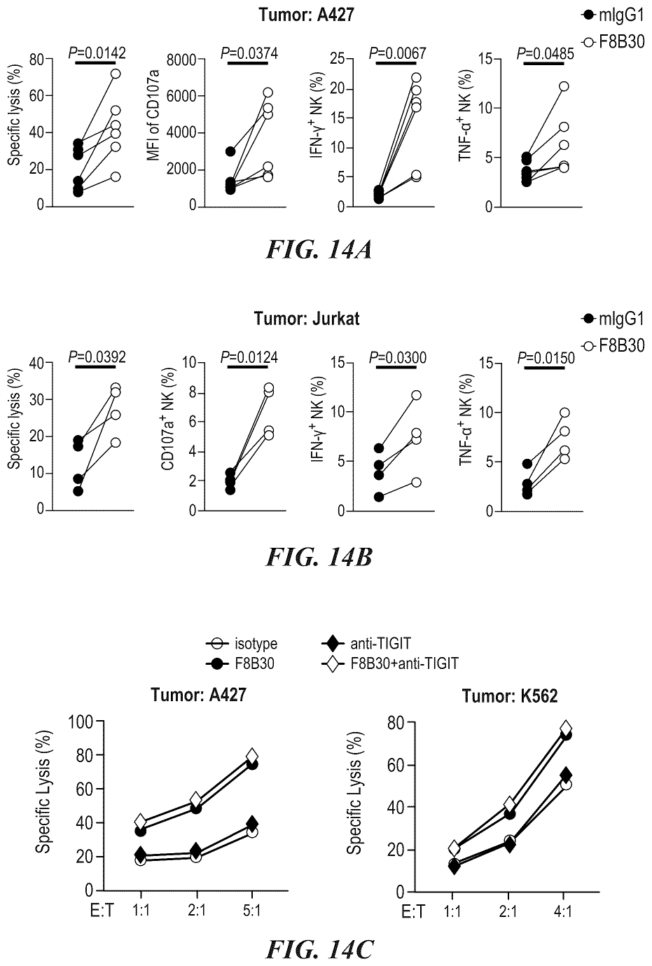

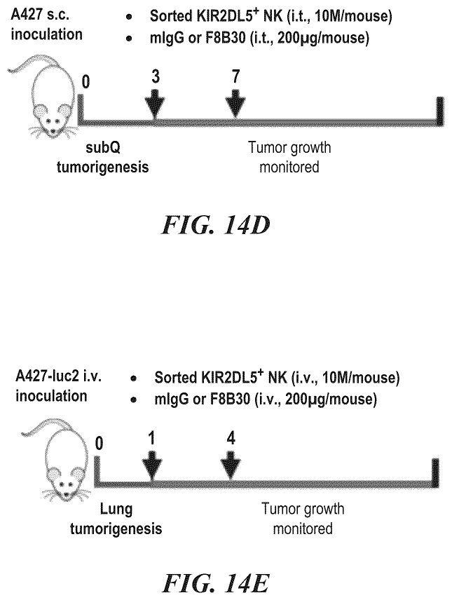

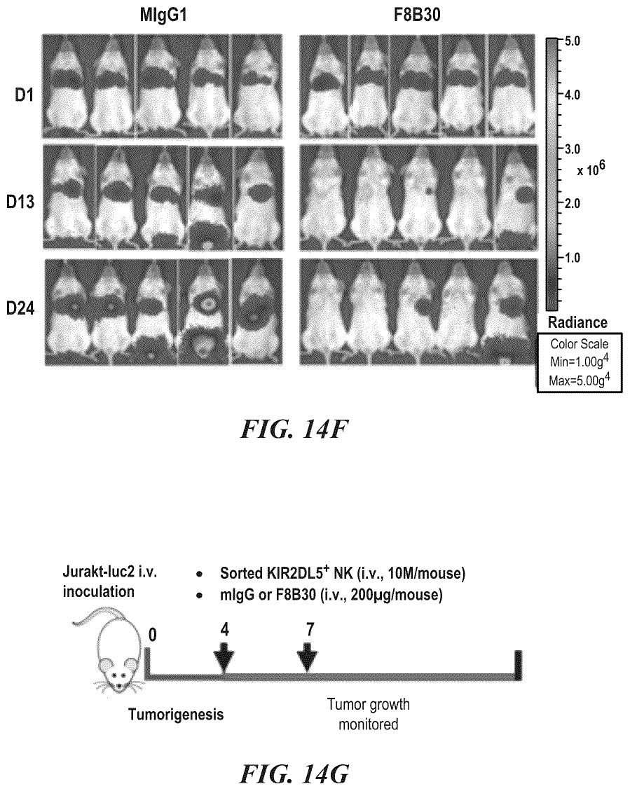

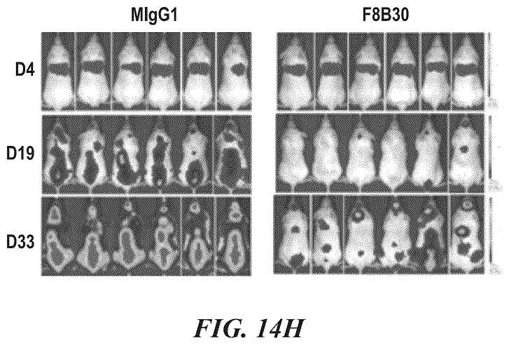

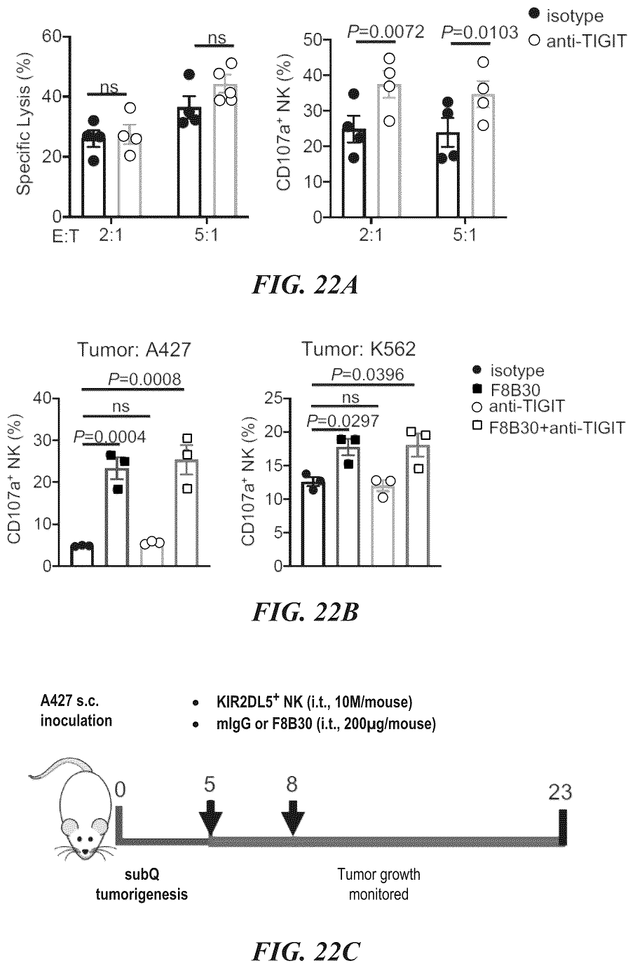

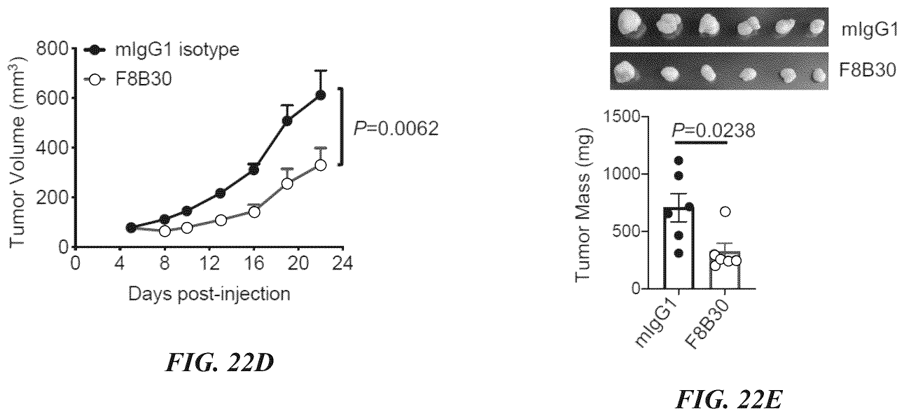

Specifically, upon incubation with anti-KIR2DL5 blocking mAb F8B30, KIR2DL5⁺ NK cells manifested more potent cytotoxicity, degranulation (CD107a), and functional cytokine (IFN-γ and TNF-α) production after coculturing with PVR⁺ A427 (FIG. 14A) or Jurkat tumor cells (FIG. 14B). TIGIT expression was low in resting NK cells but elevated upon activation with IL-2 and IL-15 (FIG. 9E). Blockade of TIGIT on activated NK cells promotes NK degranulation (FIG. 22A), confirming its inhibitory role in regulating NK cell functions. Despite the high expression of TIGIT on KIR2DL5⁺ NK cells (FIG. 19B), it was demonstrated no change in NK cytotoxicity when TIGIT alone was blocked. Enhanced tumor lysis and NK degranulation were only observed when KIR2DL5 was blocked, either alone or with TIGIT blockade (FIGS. 14C and 22B), suggesting that KIR2DL5 has a dominant role over TIGIT in inhibiting KIR2DL5⁺TIGIT⁺ NK cell cytotoxicity.

The enhancement of NK cell function by KIR2DL5 blockade recapitulated in vivo was also investigated. Humanized nonobese diabetic (NOD) was used since murine does not express a KIR2DL5 homolog. Cg-Prkdc^{scid}Il2rg^{tm1Wjl}/SzJ (NSG) mouse models. A subcutaneous tumor model was initially used, in which NSG mice were engrafted with A427 cells and then reconstituted with KIR2DL5⁺ primary NK cells intratumorally, followed by F8B30 or isotype control treatment (FIG. 14D). Compared with mIgG1 treatment, blockade of KIR2DL5 significantly inhibited tumor growth, as shown by significantly lower tumor volume (FIG. 6A) and improved overall mouse survival (FIG. 6B). Similar results were obtained using NSG-hIL-15 mice, which express human IL-15 and better support human NK cell survival after cell transfer (FIGS. 22C-22E).

Next, the antitumor efficacy of F8B30 in a more physiologically relevant lung tumor model was tested. NSG mice were inoculated i.v. with luciferase⁺ A427 tumor cells (A427-luc2) and treated with KIR2DL5⁺ primary NK cells and F8B30 or mIgG1 (FIG. 14E). Tumor growth in the lungs was monitored by bioluminescence. Compared with mIgG1-treated mice, F8B30-treated mice showed significantly slower tumor growth (FIGS. 14F and 6C). Whereas all mIgG1-treated mice reached an endpoint within 40 days, 2 of 5 F8B30-treated mice were tumor free beyond 70 days upon tumor inoculation (FIG. 6D). In the Jurkat-luc2 tumor model, F8B30 significantly reduced tumor dissemination and prolonged overall mouse survival after tumor inoculation and adoptive KIR2DL5⁺ NK cell transfer (FIGS. 14G-14H, and 6E-6F).

Taken together, these results demonstrate that KIR2DL5-PVR blockade restores the effector function of immune cells and promotes anti-tumor immunity in vitro and in vivo.

### Example 6: KIR2DL5-Induced Inhibitory Signaling in NK Cells

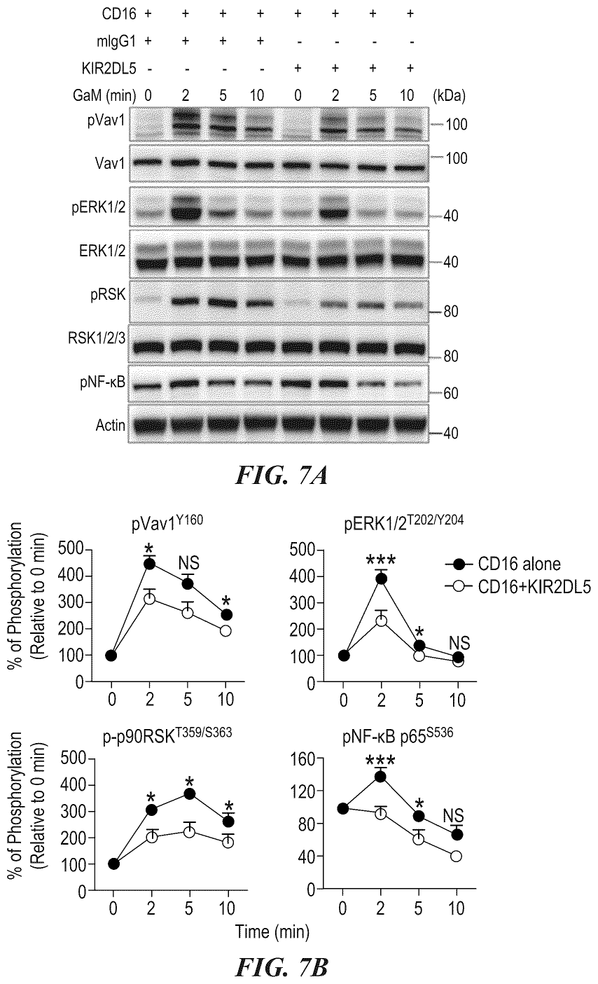

KIR2DL5-induced downstream signaling pathways were investigated in human NK cells. Upon KIR2DL5 signaling initiation, a substantially reduced activation of Vav1, ERK1/2, RSK, and NF-kB was observed in CD16-stimulated KIR2DL5+ primary NK cells (FIGS. 7A, 7B), indicating that KIR2DL5 induces inhibitory signaling in human NK cells.

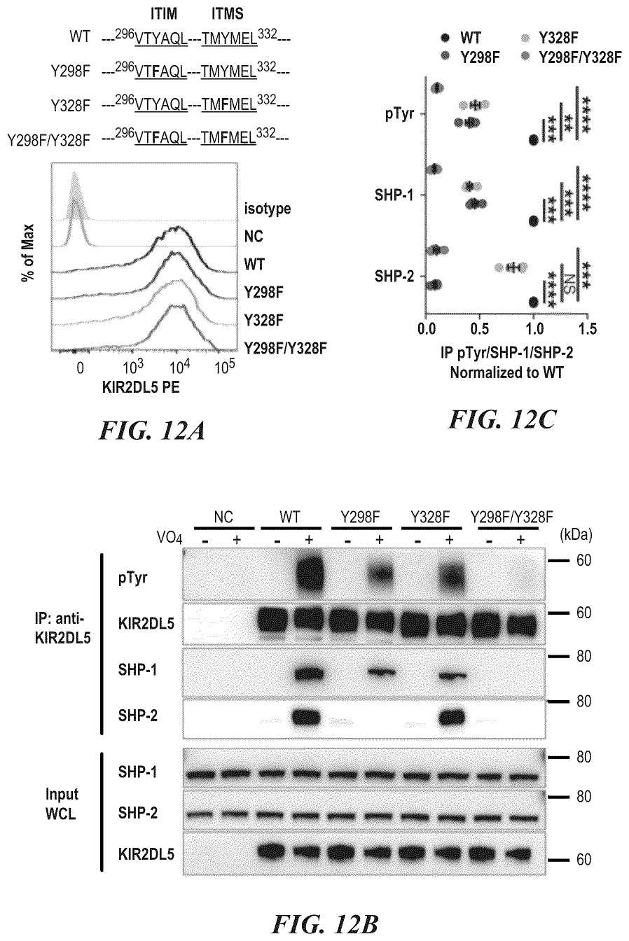

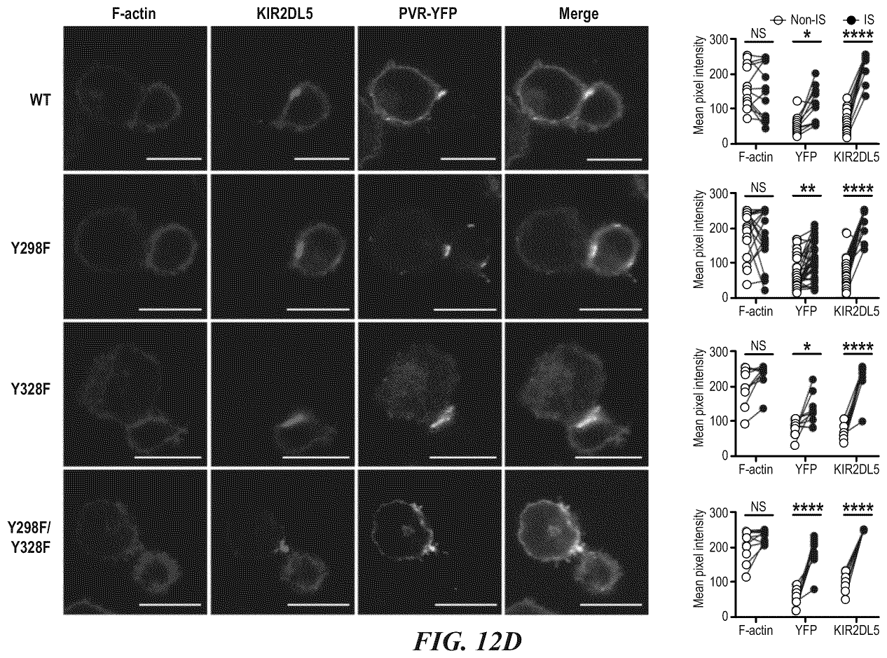

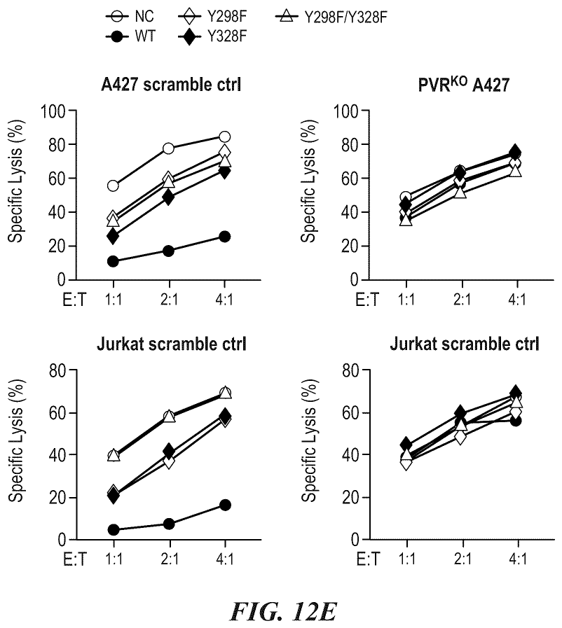

As shown in FIG. 15, the cytoplasmic tail of KIR2DL5 possesses a classical ITIM and an ITSM. The tyrosine residues were mutated into phenylalanine in the ITIM (Y298F) or ITSM (Y328F) or both (Y298F/Y328F) (FIG. 12A). WT KIR2DL5 and mutated KIR2DL5 were transduced into KIR2DL5⁻ primary NK cells, and their expression levels were similar after cell sorting (FIG. 12A). Upon treatment with the tyrosine phosphatase inhibitor pervanadate (Huyer 1997), WT KIR2DL5 exhibited tyrosine phosphorylation, whereas these mutants displayed diminished or even abrogated tyrosine phosphorylation (FIGS. 12B and 12C). It was validated that both SHP-1 and SHP-2 were recruited by WT KIR2DL5 in primary NK cells (FIG. 12B) (Estefania 2007; Yusa 2004). Notably, it was found that the KIR2DL5 association with SHP-1 was impaired by the tyrosine mutation in either ITIM or ITSM (FIGS. 12B and 12C). SHP-2 recruitment by KIR2DL5 was completely abolished by ITIM tyrosine mutation, whereas it was not altered by ITSM tyrosine mutation (FIGS. 12B and 12C). As shown in FIG. 12D, these mutations did not affect the clustering of KIR2DL5 with PVR at the interface of immunological synapses. However, PVR-KIR2DL5 interaction-mediated inhibition of NK cytotoxicity was impaired when ITIM or ITSM alone, or both, were mutated (FIG. 12E).

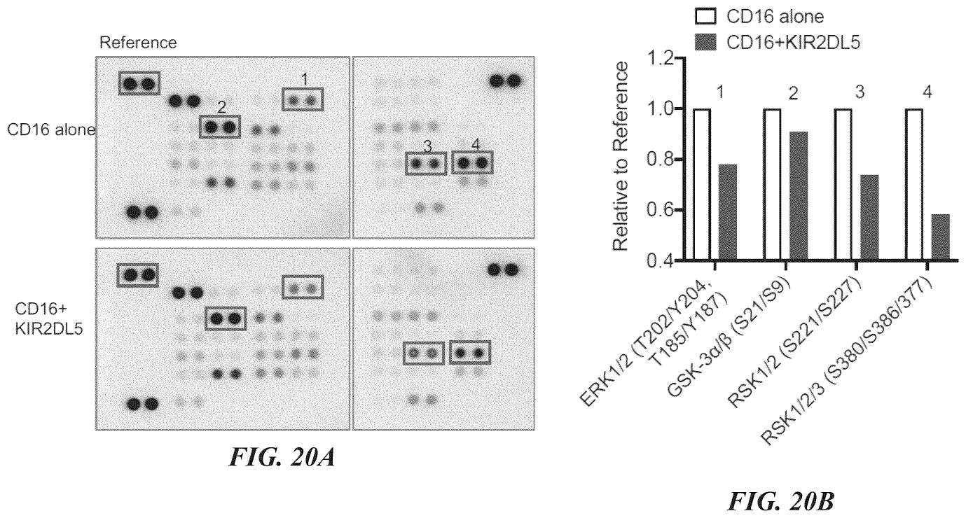

A receptor cross-linking assay was conducted to initiate KIR2DL5 signaling in CD16-stimulated primary NK cells and then subjected them to a human phospho-kinase array. Compared with CD16 alone, coengagement of KIR2DL5 with CD16 displayed a reduced phosphorylation level of multiple kinases, including ERK1/2 and p90RSK (FIGS. 20A and 20B). Further immunoblot analysis showed decreased activation of Vav1, ERK1/2, p90RSK, and the downstream transcription factor NF-1B upon KIR2DL5 signaling initiation (FIGS. 7A and 7B).

### Example 7: KIR2DL5⁺ Immune Cells Infiltrated in Various PVR⁺ Human Cancers

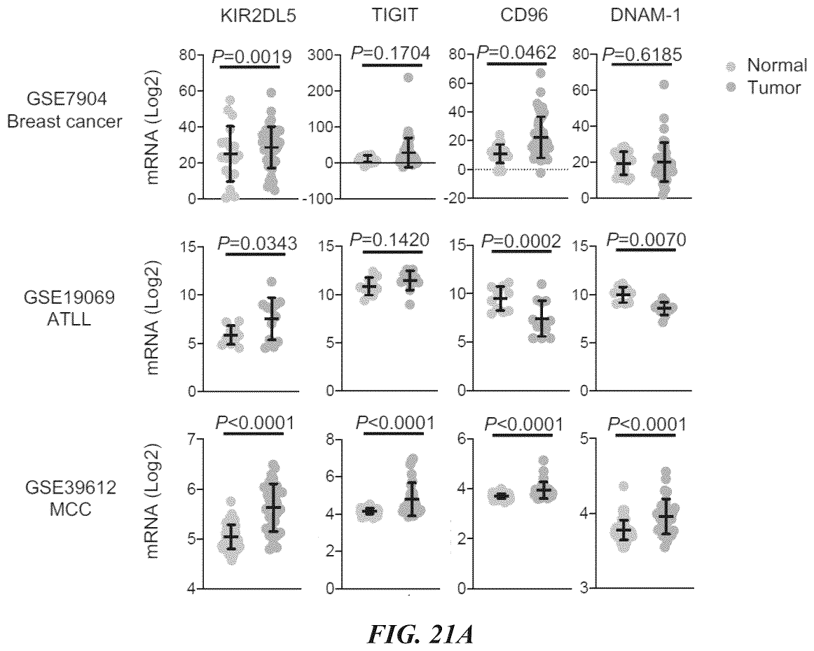

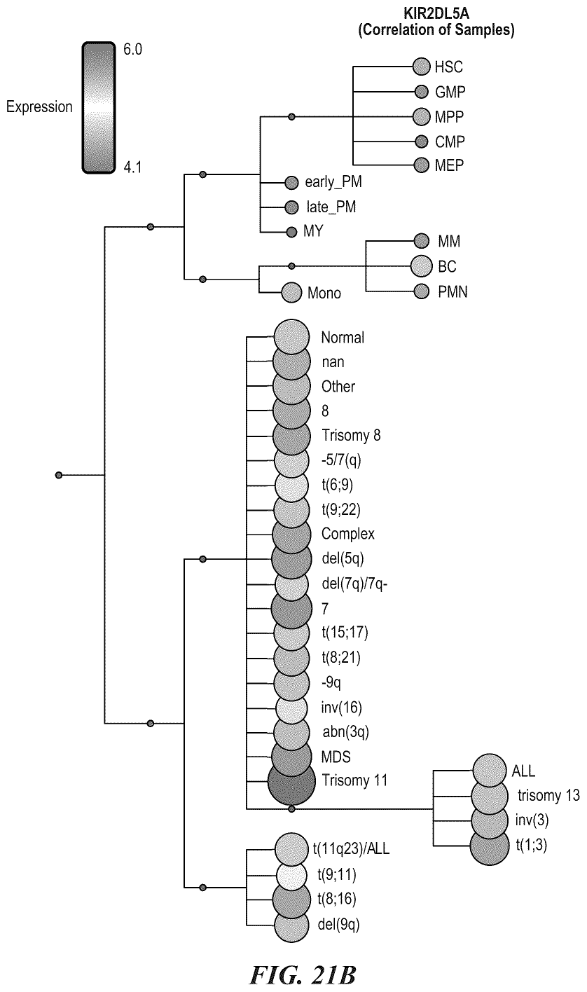

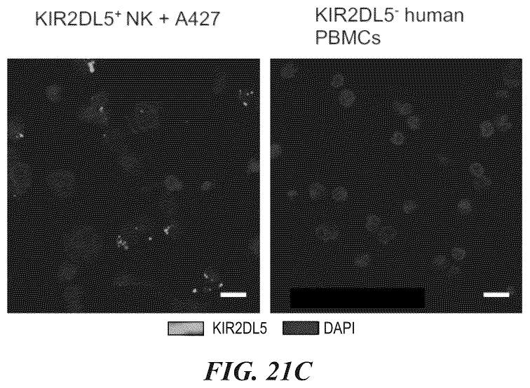

To further understand the KIR2DL5/PVR pathway within the human tumor microenvironment, data sets from the Gene Expression Omnibus database and BloodSpot databases were analyzed. It was found that KIR2DL5A mRNA was upregulated in several human solid tumors and hematopoietic malignancies by comparison with respective normal tissues (FIGS. 21A and 21B), whereas other receptors, TIGIT, CD96, and DNAM-1, were higher, lower, or showed no difference, respectively, in these tumors compared with respective normal tissues (FIG. 21A).

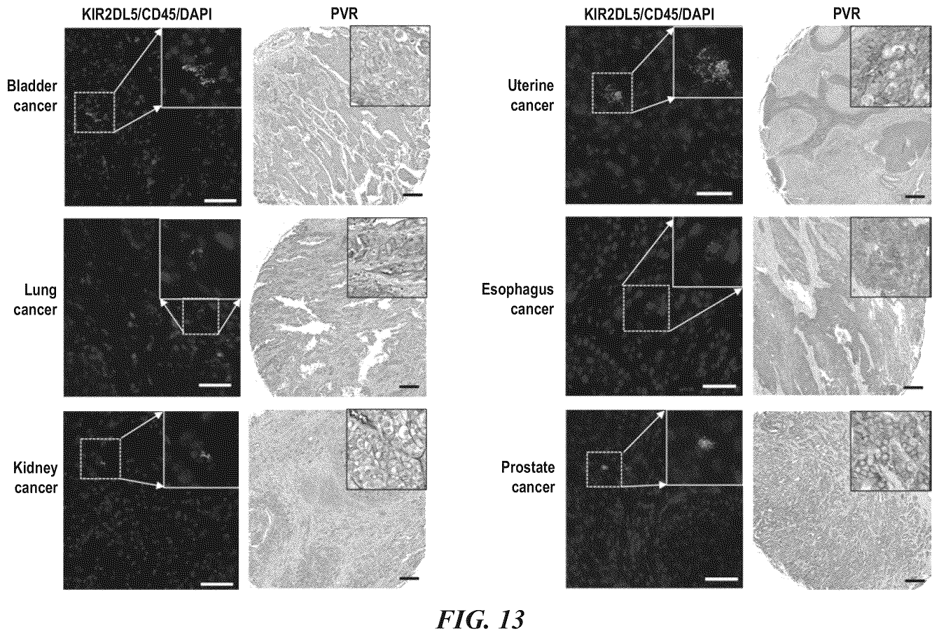

To further explore the KIR2DL5/PVR pathway in various human cancers, immunohistochemistry (IHC) staining for KIR2DL5 was initially tried, but none of the antibodies worked. RNAScope in situ hybridization was used (Niu, 2022) to examine KIR2DL5 mRNA expression on human tumor tissue microarrays (TMAs) with KIR2DL5-specific probes. The probe set for KIR2DL5A specifically stained KIR2DL5⁺ NK cells, but not KIR2DL5⁻ PBMCs (FIG. 21C). KIR2DL5⁺ CD45⁺ tumor-infiltrating immune cells were observed in a broad spectrum of human cancers (FIG. 13 and Table 4). PVR protein expression in these tumors was examined. IHC staining showed that PVR protein was widely expressed in those cancers (FIG. 13 and Table 4). These results demonstrate the presence of the immunosuppressive KIR2DL5/PVR pathway within the TME of various human cancers of bladder, kidney, breast, lung, liver, cerebrum, prostate, colon, esophagus, pancreas, uterus, and stomach, which tumors may exploit as an immune evasion mechanism.

TABLE 4
KIR2DL5 mRNA expression and PVR protein expression in human
tumor TMAs assessed by RNAScope and IHC, respectively

| KIR2DL5 mRNA expression | | | | | |
|---|---|---|---|---|---|
| in human cancers | | | PVR expression in human cancers | | |
| (number positive/total cores) | | | (number positive/total cores) | | |
| Bladder | Kidney | Breast | Bladder | Kidney | Breast |
| (17/40) | (8/19) | (4/11) | (32/40) | (19/19) | (9/11) |
| Lung | Liver | Cerebrum | Lung | Liver | Cerebrum |
| (8/20) | (3/8) | (4/11) | (17/20) | (8/8) | (9/11) |
| Prostate | Colon | Esophagus | Prostate | Colon | Esophagus |
| (6/17) | (6/19) | (5/11) | (15/17) | (17/19) | (10/11) |
| Pancreas | Uterus | Stomach | Pancreas | Uterus | Stomach |
| (4/11) | (8/19) | (3/11) | (9/11) | (18/19) | (8/11) |

Mice. BALB/c mice were purchased from Charles River Laboratory. NOD.Cg-Prkdc^{SCID}Il2rg^{tm1Wjl}/SzJ (NSG) and NSG-IL-15 mice were purchased from The Jackson Laboratory. Mice were used between 6 and 8 weeks of age. All mice were bred and maintained in a specific pathogen-free facility with a 12-hour light/12-hour dark cycle at Albert Einstein College of Medicine (Bronx, New York, USA).

Cell lines. Human cell lines used herein include Phoenix-ampho, retrovirus producer line (ATCC, CRL-3213); HEK293T, lentivirus producer line (a gift from Wenjun Guo, Department of Cell Biology, Albert Einstein College of Medicine); K562, human chronic myelogenous leukemia (ATCC, CCL-243); Jurkat, a human T lymphoblastic leukemia cell line (ATCC, TIB-152); Raji, human B cell lymphoma (ATCC, CCL-86); and A427, human lung adenocarcinoma (a gift from Haiying Cheng, Department of Cell Biology, Albert Einstein College of Medicine). These cell lines were cultured in either EMEM, DMEM, or RPMI 1640 (Gibco) medium supplemented with 10% FBS, 100 U/mL penicillin, and 100 μg/mL streptomycin. Mouse cell lines used herein were mouse fibroblast line NIH 3T3 (ATCC, CRL-1658), mouse mast cell line P815 (ATCC, TIB-64), and mouse myeloma cell line NSO (a gift from Matthew D. Scharff, Department of Cell Biology, Albert Einstein College of Medicine). Cells were cultured in DMEM supplemented with 10% FBS, 100 U/mL penicillin, and 100 μg/mL streptomycin. All cell lines were cultured at 37° C. in a humidified atmosphere containing 5% CO₂.

Human phospho-kinase arrays. The phosphorylation profiles of downstream kinases of the PVR/KIR2DL5 pathway were determined by use of a human phospho-kinase array (R&D Systems). Briefly, KIR2DL5⁺ primary NK cells (5×10⁶) were preincubated with 10 μg/mL isotype control mIgG1 or anti-KIR2DL5 mAbs (clone F8B10) in the presence of anti-CD16 (5 μg/mL) for 30 minutes on ice. After washing with medium, primary NK cells were cross-linked with 25 μg/mL goat anti-mouse IgG (minimal x-reactivity) (BioLegend) at 37° C. water bath for 2 minutes. Cells were immediately transferred to ice to stop the reaction and then lysed with cell lysis buffer, followed by analysis of the relative levels of protein phosphorylation according to the manufacturer's instructions.

Production and purification of human fusion proteins. KIR2DL5-Ig was generated in an inducible secreted serum-free Drosophila expression system as described previously (Wei 2021; Zhao 2013). Briefly, the coding region of the extracellular domain without signal peptide of KIR2DL5 was fused to a human IgG1 Fc tag in a pMT/BiP vector. Construct was cotransfected with a blasticidin-resistant plasmid into Drosophila Schneider 2 (S2) cells by the calcium phosphate transfection kit (Invitrogen). The stably transfected S2 cells were selected and expanded in Schneider's Drosophila Medium (Gibco) supplemented with 10% FBS, 100 U/mL penicillin, 100 μg/mL streptomycin, and 25 μg/mL blasticidin (Gold Biotechnology). The S2 cells were induced to secrete fusion proteins in Express Five serum-free medium (Life Technologies) in the presence of 0.75 mM CuSO₄. Proteins were purified using Protein G resin (GenScript) columns.

Generation of stable cell lines. Molecules expressed in NIH 3T3 and Raji cells were introduced by retrovirus transduction. Retrovirus was produced in Phoenix-ampho cells transfected with pCMV-VSV-G and MSCV-YFP containing the gene of interest using jetPRIME reagents (Polyplus Transfection).

Molecules expressed in A427, Jurkat, and K562 were introduced by lentiviral transduction. Lentivirus was produced in HEK293T cells transfected with pCMVR8.74, pCMV-VSV-G, and a lentiviral back-bone vector containing the gene of interest using jetPRIME reagents (Polyplus Transfection). Virus-containing supernatant was harvested 48-72 hours after transfection and filtered through a 0.45 μm filter. Cells were spin-infected at 2000 g for 120 minutes at 37° C. in the presence of 5 μg/mL Polybrene (Merck Millipore) and 1-2 mL virus supernatant. Transduced cells were sorted using a BD FACSAria Fusion Cell Sorter (BD Biosciences).

Fusion protein cell binding assays. PVR-Ig, CD112-Ig, or hIgG (R&D Systems) was incubated with corresponding 3T3 cells on ice for 45 minutes, followed by incubation with APC- or PE-conjugated anti-human IgG Fc antibody (1:100; clone HP6017, BioLegend) on ice for 30 minutes. Cells were then acquired on an LSR II flow cytometer (BD Biosciences). In the anti-KIR2DL5 mAb blocking assay, KIR2DL5/3T3 cells were preincubated with a serial concentration of anti-KIR2DL5 mAb F8B30 or mIgG1 on ice for 30 minutes. After washing, the cells were then incubated with 20 μg/mL PVR-Ig or hIgG on ice for 45 minutes, followed by incubation with APC anti-human IgG Fc antibody on ice for 30 minutes. In the PVR receptor competition binding assay, PVR-YFP/3T3 cells were preincubated with recombinant human DNAM-1-His (R&D Systems), TIGIT-His (R&D Systems), or CD96-His (Thermo Fisher Scientific) tag proteins at indicated concentrations at room temperature (RT) for 40 minutes. KIR2DL5-Ig (20 μg/mL) protein was then incubated with PVR-YFP/3T3 cells on ice for 45 minutes, followed by PE anti-human IgG Fc (1:200; BioLegend) on ice for 30 minutes. In the reverse direction, PVR-Ig protein (20 μg/mL) was preincubated with indicated concentrations of His-tagged protein and then stained KIR2DL5/3T3 cells on ice for 45 minutes, followed by PE anti-human IgG Fc on ice for 30 minutes. Cells were then acquired on an LSR II (BD Biosciences).

Intercellular conjugation assay. PVR/3T3 and HHLA2/3T3 cells were prelabeled with eFluor 450 (eBioscience) while KIR2DL5/3T3 and KIR3DL3/3T3 were prelabeled with PKH26 (Sigma-Aldrich) to distinguish from each other. PVR/3T3 or HHLA2/3T3 cells (2×10⁵) were then incubated with KIR2DL5/3T3 or KIR3DL3/3T3 (2×10⁵) at 37° C. for 45 minutes. In the mAb blocking assay, PVR/3T3 cells were coincubated with KIR2DL5/3T3 or KIR3DL3/3T3 cells in the presence of the indicated anti-KIR2DL5 mAbs or mIgG1 (10 μg/mL). After washing, cells were acquired on an LSR II (BD Biosciences) to analyze intercellular conjugation.

Generation of mAbs against KIR2DL5. Mouse anti-KIR2DL5 mAbs were generated by hybridoma techniques as described previously (Wei 2021; Zhao 2013). Briefly, splenocytes from KIR2DL5-Ig-immunized BALB/c mice were fused with NSO myeloma cells. Eight clones that specifically recognized KIR2DL5 were selected by high-throughput flow cytometry. Hybridoma cells were cultured in CELLine 350 Bioreactor Flask (DWK Life Sciences). Antibodies were purified from hybridoma supernatant by Protein G resin (GenScript) columns. The purity and integrity of antibodies were determined by SDS-PAGE and FACS. Clone F8B30 was conjugated with PE by SiteClick R-PE Antibody Labeling Kit (Invitrogen) for the following analysis.

Biolayer interferometry. The affinities of anti-KIR2DL5 mAbs were analyzed by biolayer interferometry using an Octet RED96 system (ForteBio, Pall LLC). Briefly, anti-human Fc capture biosensors (ForteBio, Pall LLC) were preloaded with KIR2DL5-Ig and then dipped into a solution containing mAb at 2-fold serial dilutions (from 200 to 1.5 μg/mL). Data were analyzed using Forte Pall (Port Washington, New York, USA) software 9.0. The global data fitting to a 1:1 binding model was used to estimate values for the Kₒₙ (association rate constant), K_{off} (dissociation rate constant), and K_{D} (equilibrium dissociation constant).

Immunophenotyping by flow cytometry. Monoclonal antibodies (clone 26E10) against KIR3DL3 were purified in-house (Wei 2021). The following fluorophore-conjugated antibodies were used (all antibodies from BioLegend unless otherwise indicated) (see Table 5): CD3 (clone UCHT1, BD Biosciences), CD4 (clone RPA-T4), CD8 (clone RPA-T8, BD Biosciences), CD16 (clone 3G8, BD Biosciences), CD19 (clone SJ25C1), CD56 (clone 5.1H11), anti-human CD57 (clone QA17A04), TCR γδ (clone B1), CCR7 (clone G043H7), CD45RA (clone HI100), CD155 (clone SKII.4), DNAM-1 (clone 11A8), TIGIT (clone A15153G), CD96 (clone NK92.39), CD107a (clone H4A3), IFN-γ (clone B27), TNF-α (clone MAb11), CD57 (clone HNK-1), KLRG1 (clone SA231A2), KIR3DL2 (clone 539304, R&D), KIR2DL1/S1/S3/S5 (clone HP-MA4), KIR2DL2/3 (clone DX27), KIR2DL4 (clone mAb 33), KIR2DL5 (clone UP-R1), NKG2D (clone 1D11), NKG2C (clone 134591, R&D), NKG2A (clone 131411, BD Biosciences), 2B4 (clone C1.7), NKp46 (clone 9E2), NKp44 (clone p44-8, BD Biosciences), NKp30 (clone p30-15, BD Biosciences).

Human PBMCs were stained with Zombie Violet Fixable Viability Kit (BioLegend) and then incubated with FcR blocking reagents (Miltenyi Biotec). For surface marker staining, cells were incubated with specific antibodies for 30-45 minutes at 4° C. For CD107a and intracellular cytokine staining, cells were incubated with anti-CD107a in the presence of 5 μg/mL brefeldin A and 2.5 μg/mL monensin (BioLegend) for 5 hours. Cells were then fixed and permeabilized using the Fixation/Permeabilization Solution Kit (BD Biosciences) according to the manufacturer's instructions, followed by staining with intracellular antibodies for 30-45 minutes at 4° C. All samples were acquired on an LSR II (BD Biosciences) or Aurora (Cytek) and were analyzed using FlowJo software (BD Biosciences). DownSample and t-distributed stochastic neighbor embedding (t-SNE) plugins in FlowJo and ggplot2 package in R were used to generate t-SNE plots.

Isolation and culture of human NK cells. Human PBMCs were isolated from the buffy coats of healthy donors purchased from New York Blood Center, using Ficoll-Hypaque (GE Healthcare) density gradient separation. Human KIR2DL5⁺ primary NK cell were sorted by FACS and then expanded by culturing with autologous PBMCs as feeder cells (irradiated at 30 Gy, feeder cells: NK cells=20:1) in OpTimizer (Invitrogen) supplemented with 5% human AB serum (Sigma-Aldrich), 1% L-glutamine, 100 U/mL penicillin, 100 μg/mL streptomycin, anti-CD3 OKT3 (10 ng/mL; BioLegend), recombinant human IL-2 (40 ng/mL; BioLegend), and IL-15 (10 ng/mL; BioLegend). Five or six days later, NK cells were further expanded in the same medium without anti-CD3 and feeder cells.

Primary NK cell transduction. KIR2DL5 wild type and variants of ITIM/ITSM expressed on the surface of KIR2DL5⁻ primary NK cells were introduced by lentiviral transduction. Lentivirus was produced in HEK293T cells cotransfected with psPAX, pMD2.G, and a lentiviral backbone pSin vector (a gift from the Alec Zhang laboratory, Department of Physiology, University of Texas Southwestern Medical Center, Dallas, Texas, USA) containing the full-length gene sequence of KIR2DL5A*001 using jetPRIME reagents (Polyplus Transfection). Virus-containing supernatant was harvested 48-72 hours after transfection and filtered through a 0.45 m filter. Non-tissue-culture-treated plates were coated with retronectin, and virus supernatant was then incubated on the surface of plates at 2,000 g for 120 minutes at 37° C. NK cells were subsequently spun down at 1000 g for 10 minutes at 37° C. Transduced NK cells were sorted using a BD FACSAria Fusion Cell Sorter (BD Biosciences).

Cytotoxicity assay. Cytotoxicity assays were performed through a flow-based assay. Briefly, target cells were labeled with PKH26 (Sigma-Aldrich) for 2 minutes at 37° C. For mAb blocking assay, primary NK cells were preincubated with 20 μg/mL of mIgG1, anti-KIR2DL5 mAb (clone F8B30), anti-TIGIT mAbs (clone MBSA43, eBioscience), or indicated combination for 30 minutes before coculture with target cells.

In CD16-induced redirected cytotoxicity assays, anti-human CD16 mAbs (clone 3G8) were used to activate NK cells through cross-linking CD16. Briefly, P815 cells were preincubated with 0.5 μg/mL of anti-human CD16 and 2 μg/mL of mIgG1, anti-KIR2DL5 mAb (clone F8B30), or anti-CD56 (clone 5.1H11) for 15 minutes at RT. Target cells were coincubated with effector NK cells in 96-well round-bottom plates at indicated E/T ratios for 4-6 hours at 37° C. Supernatants from redirected cytotoxicity assays were collected after 24 hours of coculture for Human Cytokine 65-Plex Assay (Eve Technologies). 7-AAD was used to differentiate dead cells from live cells. The standard formula of 100×PKH26⁺7-AAD⁺ cells/PKH26⁺ cells % was used to calculate specific lysis percentages.

NK-Raji conjugation assay. KIR2DL5⁺ primary NK or transduced NK cells (5×10⁵) with KIR2DL5 WT, Y298F, Y328F, or Y298/328F mutants were coincubated with 5×10⁵ PVR-YFP/Raji or YFP/Raji cells in a 50 mL tube at 37° C. for 40 minutes. Cell mixtures were then loaded onto poly-L-lysine-precoated slides and fixed with 4% formaldehyde at RT for 15 minutes. After blocking with 5% normal goat serum at RT for 1 hour, cells were stained with 20 g/mL anti-KIR2DL5 antibodies (a mixture of 8 homemade clones) at 4° C. overnight and then with goat anti-mIgG (H+L) Alexa Flour 647 (Invitrogen) at RT for 2 hours. The cells were permeabilized by 0.1% Triton X-100 at RT for 15 minutes and stained with Alexa Flour Plus 405 Phalloidin (Life Technologies) for 1 hour at RT. The slides were then mounted by Gold Antifade Mountant without DAPI (Life Technologies). The mean pixel intensity of synapse and non-synapse was respectively measured and statistically analyzed. Images were acquired by Leica SP8 confocal microscope and processed by ImageJ (NIH).

Plasmid construction and site-directed mutagenesis. The plasmid encoding KIR2DL5 was purchased from Molecular Cytogenetics Core of Albert Einstein College of Medicine, and the fragment of KIR2DL5 was inserted into MSCV-YFP vector. The mutagenesis was carried out using New England Biolabs Q5 Site Directed Mutagenesis Kit.

The mutants of KIR2DL5 were constructed using the following primers: deleted DO forward, GGTCTATTTGGGAAACCTTCACTCTCAG (SEQ ID NO:34); deleted DO reverse, TGTCCAGGCCCCCTGCAG (SEQ ID NO:35); deleted D2 forward, GGAAACTCTTCAAGTAGTTCATC (SEQ ID NO:36); deleted D2 reverse, TGTGACCACGATCACCAG (SEQ ID NO:37); N173D forward, GCCCAGCGTCGATGGAACATTCC (SEQ ID NO:38); N173D reverse, ACTGCAGGGAGCCTAGGTT (SEQ ID NO:39); N173D/G195S for 2DL5A*005 forward, CACATGCTTCAGCTCTCTCCATGAC (SEQ ID NO:40); N173D/G195S for 2DL5A*005 reverse, TAGGTCCCTCCGTGGGTG (SEQ ID NO:41); I6V forward, GCTCATGGTCGTCAGCATGGCGT (SEQ ID NO:42); I6V reverse, GACATAGATCTAATCCGGCGC (SEQ ID NO:43); I6V/T21P for 2DL5B*00602 forward, GGGGGCCTGGCCACATGAGGGTG (SEQ ID NO:44); I6V/T21P for 2DL5B*00602 reverse, TGCAGCAAGAAGAACCCAACACAC (SEQ ID NO:45); I6V/T21P/V116M for 2DL5B*003 forward, CCTGGTGATCATGGTCACAGGTC ((SEQ ID NO:46); I6V/T21P/V116M for 2DL5B*003 reverse, GGGTTGCTGGGTGCTGAC (SEQ ID NO:47); T46S forward, GGACATGTGAGTCTTCTGTGTCGC (SEQ ID NO:48); T46S reverse, TCCTCGAGGCACCACAGC (SEQ ID NO:49); R52H forward, TGTCGCTCTCATCTTGGGTTTAC (SEQ ID NO:50); R52H reverse, CAGAAGAGTCACATGTCC (SEQ ID NO:51); G97S forward, CAGATGTCGGAGTTCACACCCAC (SEQ ID NO:52); G97S reverse, TAGGTCCCTGCGTGTGCA (SEQ ID NO:53); P112S forward, ACCCAGCAACTCCCTGGTGAT (SEQ ID NO:54); P112S reverse, GCTGACCACTCAATGGGG (SEQ ID NO:55); Y298F forward primer, GGAGGTGACATTTGCACAGTTGG (SEQ ID NO:56); Y298F reverse primer, TGAGGGTCTTGATCATCAG (SEQ ID NO:57); Y328F forward primer, TACCACCATGTTCATGGAACTTC (SEQ ID NO:58); Y328F reverse primer, TCTGTTGGAGGTGTCTTG (SEQ ID NO:59).

The following primers were used to construct pSin-KIR2DL5 WT vector and mutants:

pSin forward,
(SEQ ID NO: 60)
TGTCGTGAGGAATTGATCCTTCGAACTAGTATGTCGCTCATGGTCATCA
G;
pSin reverse primer,
(SEQ ID NO: 61)
TGTAAGTCATTGGTCTTAAAGGTACCTGAGGTCAGATTCCAGCTGCTGG
T.

The restriction enzyme sites were Bsu36I and SpeI.

Lentiviral CRIPR/Cas9-induced deletion of PVR. The scramble control sgRNA and PVR-targeting sgRNA were designed using GPP sgRNA Designer (Doench 2016) (https://portals.broadinstitute.org/gpp/public/analysis-tools/sgrna-design). Oligonucleotides were annealed in T4 DNA-ligase buffer (New England Biolabs), cloned into lentiCRISPR version 2 (Addgene, 52961).

The sgRNA sequences were as follows: scrambled control sgRNA: 5′-GCACTACCAGAGCTAACTCA-3′ (SEQ ID NO:62); PVR targeting sgRNA no. 1: 5′-GATGTTCGGGTTGCGCGTAG-3′ (SEQ ID NO:63); PVR targeting sgRNA no. 2: 5′-TTGAGGGCACCAATATCCAG-3′ (SEQ ID NO:64).

All these constructs are not predicted to target any known sequences in the human genome. The lentiviruses were produced as described above. A427 and K562 were transduced with viral supernatant and then selected by puromycin (2 μg/mL) for 3 days. Stable knockout of PVR (PVR KO) was confirmed by flow cytometry analysis.

Coimmunoprecipitation and immunoblotting. NK92 cells or primary NK cells pretreated with or without 1 mM pervanadate (New England BioLabs) were lysed in Pierce immunoprecipitation lysis buffer supplemented with protease and phosphatase inhibitor cocktail (Thermo Fisher Scientific). Proteins from whole-cell lysis were further incubated with anti-KIR2DL5 antibodies and Dynabeads protein G (Thermo Fisher Scientific) for further immunoprecipitation. To analyze phosphorylation status, after receptor cross-linking, the cells were lysed in radioimmunoprecipitation lysis buffer (50 mM Tris-HCl [pH 7.5], 0.15 M NaCl, 1% NP-40, 0.5% sodium deoxycholate, and 0.1% SDS) supplemented with protease and phosphatase inhibitor cocktail. Samples were separated on SDS-PAGE gels (GenScript) and transferred onto nitrocellulose membranes (Bio-Rad) for protein detection.

The following antibodies were used: anti-phospho-tyrosine 4G10 (1:1,000; Sigma-Aldrich), anti-SHP-1 (1:500; Cell Signaling Technology [CST]), anti-SHP-2 (1:500; CST), anti-Vav1 (1:2,000; CST), anti-phospho-Vav1 Tyr160 (1:2,000; Invitrogen), anti-ERK1/2 (1:2,000; CST), anti-phospho-ERK1/2 Thr202/Tyr204 (1:1000; BioLegend), anti-p90RSK (1:1,000; CST), anti-phospho-p90RSK Thr359/Ser363 (1:1000; CST), anti-phospho-NF-κB p65 Ser536 (1:1,000; CST), anti-3-actin (1:2,000; Santa Cruz Biotechnology), HRP-conjugated goat anti-mouse (1:1,0000; Jackson ImmunoResearch), rabbit anti-goat (1:1,0000; Jackson ImmunoResearch), and goat anti-rabbit (1:2,000; CST) secondary antibodies and enhanced chemiluminescent substrate (ECL; Bio-Rad).

RNAScope in ISH and imaging. RNAScope ISH for KIR2DL5 and CD45 mRNA expression in FFPE human tumor tissue microarrays (TMAs; US Biomax) was performed with RNAScope 2.5 HD Reagent kit (Advanced Cell Diagnostics) per the manufacturer's instructions (Wang 2012). Briefly, TMA slides were deparaffinized, subjected to antigen retrieval using citrate buffer for 15 minutes at a boiling temperature, and then treated with 10 μg/mL protease at 40° C. for 30 minutes. Probes were hybridized for 2 hours at 42° C. followed by signal amplification. For fluorescent detection, the label probe sets for KIR2DL5 and CD45 were conjugated to Opal 570 and 690 nm (Akoyo Biosciences), respectively. Assays were typically performed in parallel with positive (UBC) and negative (bacterial gene dapB) controls to assess both tissue RNA integrity and background signals. The slides were scanned by a 3DHistech P250 high-capacity slide scanner by 3 channels with filter settings for DAPI, FITC, and Cy7. Staining was analyzed with Volocity software by a trained researcher.

IHC staining and imaging. The same cohorts of TMAs used in RNAScope ISH were deparaffinized, followed by antigen retrieval with citrate unmasking buffer (CST) in a steamer for 20 minutes at a sub-boiling temperature (95° C.-98° C.). Slides were then blocked by 3% hydrogen peroxidase solution at RT for 10 minutes and subsequently by 10% normal goat serum at RT for 1 hour. A rabbit anti-PVR (clone D8A5G, CST) mAb was used at a dilution of 1:200 for overnight incubation at 4° C. The slides were then incubated with boost detection reagent (HRP, CST) at RT for 30 minutes, followed by SignalStain DAB (CST) and hematoxylin nuclear counterstaining. Positive and negative controls (FFPE cell blocks) were included in each staining.

Xenograft models of human cancers. For the subcutaneous A427 tumor model, 6- to 8-week-old NSG or NSG-hIL-15 mice were inoculated s.c. with 3×10⁶ A427 cells on the hind flanks. Three or five days later, mice were randomized into 2 groups (n=6 or 8) and treated with KIR2DL5⁺ primary NK cells (1×10⁷) and 200 μg anti-KIR2DL5 mAb (clone F8B30) or isotype control (mIgG1) intratumorally twice (once every 3 days). Tumors were measured by caliper, and tumor volume was calculated as (width²×length)/2.

For the intravenous A427 tumor model, NSG mice were injected intravenously (i.v.) with 1×10⁶ luciferase-expressing A427 cells (A427-luc2). One day later, mice underwent bioluminescence imaging (BLI) and were allocated to 2 groups (n=5) based on similar average photon flux (photons/second). Mice were then treated i.v. with KIR2DL5⁺ primary NK cells (1×10⁷) and 200 μg F8B30 or mIgG1 twice (once every 3 days). Lung tumor growth was monitored by BLI weekly, and mice were euthanized when the total flux reached to 1×10⁸ photons/second.

For the intravenous Jurkat tumor model, NSG mice were injected i.v. with 5×10⁵ luciferase-expressing Jurkat cells (Jurkat-luc2). Four days later, mice were allocated to 2 groups (n=4 or 6) based on similar average photon flux (photons/second), and treated i.v. with KIR2DL5⁺ primary NK cells (1×10⁷) and 200 μg F8B30 or mIgG1 twice (once every 3 days).

Tumor growth was monitored by BLI, and mice were euthanized when the total flux reached to 1×10¹⁰ photons/second. For all BLI, D-luciferin (150 mg/kg; Gold Biotechnology) was administered by intraperitoneal injection to mice for 10 minutes before imaging. The data were analyzed with Living Image 3.0 software.

Data availability and statistical analysis. Previously published Gene Expression Omnibus (GEO) data that were reanalyzed here are available under accession codes GSE7904, GSE19069, and GSE39612.

Statistical analyses were performed in GraphPad Prism, version 9.0 (GraphPad Software) using appropriate tests as indicated in the figure legends (unpaired 2-tailed t test, paired 2-tailed t test, 1-way ANOVA followed by Tukey's or Dunnett's multiple-comparison test, 2-way ANOVA followed by Šidák's multiple-comparison test, multiple t test, and log-rank test for Kaplan-Meier survival curves). The data are expressed as mean±SEM of n=3 or more determinations. A P value of less than 0.05 was considered statistically significant.

TABLE 5
Antibodies used in the Examples

| Antibodies | Source | Identifier |
|---|---|---|
| Anti-human IgG Fc APC (clone | Biolegend | Cat# 409306; |
| HP6017) | | RRID:AB_11150591 |
| Anti-human IgG Fc PE (clone HP6017) | Biolegend | Cat# 409303 |
| | | RRID:AB 10900424 |
| F(ab′)2-goat anti-mouse IgG APC | eBiocience | Cat# 17-4010-82; |
| (polyclonal antibody) | | RRID:AB_2573203 |
| Goat anti-mouse IgG PE (polyclonal | Biolegend | Cat# 405307; |
| antibody) | | RRID:AB_315010 |
| Mouse IgG1 isotype (clone HKSP) | Leinco | Cat# I-536; |
| | Technologies | RRID:AB_2737545 |
| Anti-human CD3-BUV805 (clone | BD | Cat# 612895; |
| UCHT1) | | RRID:AB_2739277 |
| Anti-human CD4-Alexa 700 (clone | Biolegend | Cat# 300526; |
| RPA-T4) | | RRID:AB_493743 |
| Anti-human CD8-BUV563 (clone | BD | Cat# 612914; |
| RPA-T8) | | RRID:AB_2744461 |
| Anti-human CD56-PE/Cy5 (clone | Biolegend | Cat# 362516; |
| 5.1H11) | | RRID:AB_2564089 |
| Anti-human CD57-BV510 (clone | Biolegend | Cat# 393313; |
| QA17A04) | | RRID:AB_2750341 |
| Anti-human TCR γδ-PE (clone B1) | Biolegend | Cat# 331210; |
| | | RRID:AB_1089218 |
| Anti-human CCR7-BV750 (clone | Biolegend | Cat# 353253; |
| G043H7) | | RRID:AB_2800944 |
| Anti-human CD45RA-BV570 (clone | Biolegend | Cat# 304131; |
| HI100) | | RRID:AB_10897946 |
| Anti-human PVR-PE (clone SKII.4) | Biolegend | Cat# 337609; |
| | | RRID:AB_2253258 |
| Anti-human DNAM-1-FITC (clone | Biolegend | Cat# 338303; |
| 11A8) | | RRID:AB_1279145 |
| Anti-human TIGIT-APC (clone | Biolegend | Cat# 372705; |
| A15153G) | | RRID:AB_2632731 |
| Anti-human CD96-PerCP/Cy5.5 | Biolegend | Cat# 338411; |
| (clone NK92.39) | | RRID:AB_2566143 |
| Anti-human CD16-BUV496 (clone | BD | Cat# 612945; |
| 3G8) | | RRID: AB_2744294 |
| Anti-human CD19-BUV395 (clone | BD | Cat# 563551; |
| SJ25C1) | | RRID:AB_2738272 |
| Anti-human KLRG1-APC (clone | Biolegend | Cat# 367716; |
| SA231A2) | | RRID:AB_2572161 |
| Anti-human NKp46-Alexa 647 (clone | Biolegend | Cat# 331909; |
| 9E2) | | RRID:AB_1027674 |
| Anti-human KIR3DL2-Alexa 700 | R&D | Cat# FAB2878N025 |
| (clone 539304) | | |
| Anti-human KIR3DL3-PE (clone | Zang lab | Wei, et al. 2021 |
| 26E10) | | |
| Anti-human NKG2D-APC/Cy7 (clone | Biolegend | Cat# 320824; |
| 1D11) | | RRID:AB_2566660 |
| Anti-human KIR2DL1/S1/S3/S5- | Biolegend | Cat# 339511; |
| PE/Cy7 (clone HP-MA4) | | RRID:AB_2565578 |
| Anti-human NKG2C-Alexa 488 (clone | R&D | Cat# FAB138G025; |
| 134591) | | RRID:AB_10890779 |
| Anti-human KIR2DL2/L3- | Biolegend | Cat# 312613; |
| PerCP/Cy5.5 (clone DX27) | | RRID:AB_2564334 |
| Anti-human KIR2DL4-APC (clone | Biolegend | Cat# 347007; |
| mAb 33) | | RRID: AB_2249479 |
| Anti-human 2B4-BV605 (clone C1.7) | Biolegend | Cat# 329535; |
| | | RRID:AB_2814197 |
| Anti-human NKG2A-BV650 (clone | BD | Cat# 747920; |
| 131411) | | RRID:AB_2872381 |
| Anti-human NKp44-BV711 (clone | BD | Cat# 744303; |
| p44-8) | | RRID:AB_2742133 |
| Anti-human NKp30-BV786 (clone | BD | Cat# 743172; |
| p30-15) | | RRID:AB_2741323 |
| Anti-human CD107a-Alexa 488 (clone | Biolegend | Cat# 328610; |
| H4A3) | | RRID:AB_1227504 |
| Anti-human IFN-γ-PerCP/Cy5.5 | Biolegend | Cat# 506528; |
| (clone B27) | | RRID:AB_2566187 |
| Anti-human TNF-α-PE/Cy7 (clone | Biolegend | Cat# 502930; |
| mab11) | | RRID:AB_2204079 |
| Anti-human KIR2DL5-PE (clone UP- | Biolegend | Cat# 341303; |
| R1) | | RRID:AB_1595545 |
| Anti-human KIR2DL5-PE (clone | Disclosed herein | N/A |
| F8B10) | | |
| Anti-human KIR2DL5 (clone F8B30) | Disclosed herein | N/A |
| Anti-human KIR2DL5 (clone B7B23) | Disclosed herein | N/A |
| Anti-human KIR2DL5 (clone B33C12) | Disclosed herein | N/A |
| Anti-human KIR2DL5 (clone E12B11) | Disclosed herein | N/A |
| Anti-human KIR2DL5 (clone B2A18) | Disclosed herein | N/A |
| Anti-human KIR2DL5 (clone G11B22) | Disclosed herein | N/A |
| Anti-human KIR2DL5 (clone B19C11) | Disclosed herein | N/A |
| Anti-human KIR2DL5 (clone B11B4) | Disclosed herein | N/A |
| Purified anti-human CD3 antibody | Biolegend | Cat# 317326; |
| (clone OKT3) | | RRID:AB_11150592 |
| Purified anti-human CD56 (clone | Biolegend | Cat# 362502; |
| 5.1H11) | | RRID:AB_2563558 |
| Purified anti-human CD16 antibody | Biolegend | Cat# 302014; |
| (clone 3G8) | | RRID:AB_314214 |
| Purified anti-human DNAM-1 antibody | BD | Cat# 559787; |
| (clone DX11) | | RRID:AB_397328 |
| Purified anti-human TIGIT antibody | eBiocience | Cat# 16-9500-82; |
| (clone MBSA43) | | RRID:AB_10718831 |
| Anti-PVR (clone D8A5G) | Cell Signaling | Cat# 81254S; |
| | Technology | RRID: AB_2799970 |
| Anti-phosphotyrosine antibody (clone | Merck Millipore | Cat# 05321; |
| 4G10) | | RRID:AB_309678 |
| Anti-β-actin (clone C11) | Santa Cruz | Cat# sc-1615; |
| | | RRID:AB_630835 |
| Anti-phospho ERK1/2 | Biolegend | Cat# 369502; |
| (Thr202/Tyr204) (clone 6B8B69) | | RRID:AB_2721735 |
| Anti-total ERK1/2 (clone 137F5) | Cell Signaling | Cat# 4695T; |
| | Technology | RRID:AB_2339400 |
| Anti-phospho Vav1 (Tyr160) | Invitrogen | Cat# 44-482; |
| (polyclonal antibody) | | RRID:AB_2533661 |
| Anti-total Vav1 (clone D45G3) | Cell Signaling | Cat# 4657S; |
| | Technology | RRID:AB_10624865 |
| Anti-phospho-p90RSK | Cell Signaling | Cat# 9344S; |
| (Thr359/Ser363) (polyclonal antibody) | Technology | RRID:AB_915783 |
| Anti-total p90RSK (clone 32D7) | Cell Signaling | Cat# 9355S; |
| | Technology | |
| Anti-SHP-1 (clone C14H6) | Cell Signaling | Cat# 3759; |
| | Technology | RRID:AB_2173694 |
| Anti-SHP-2 (clone D50F2) | Cell Signaling | Cat# 3397; |
| | Technology | RRID:AB_2174959 |
| Anti-phospho NF-κB p65 (Ser536) | Cell Signaling | Cat# 3033S; |
| (clone 93H1) | Technology | RRID:AB_331284 |
| Goat anti-rabbit IgG-HRP | Cell Signaling | Cat# 7074S; |
| | Technology | RRID:AB_2099233 |
| Goat anti-mouse IgG-HRP | Jackson | Cat# 115-035-003; |
| | ImmunoResearch | RRID:AB_10015289 |
| Rabbit anti-goat IgG-HRP | Jackson | Cat# 305-035-003; |
| | ImmunoResearch | RRID:AB_2339400 |

Discussion Human KIRs are critical regulators of NK cell function and are important for immunological tolerance and tumor surveillance (Pende 2019). KIR2DL5 is the most recently identified KIR molecule (Estefania 2007). A nectin/nectin-like family protein, PVR, was recently identified as a binding partner for KIR2DL5 (Verschueren 2020; Husain 2019).

The present examples demonstrate that KIR2DL5 suppresses primary NK cell cytotoxicity against multiple solid and hematopoietic tumor cells in a PVR-dependent manner. KIR2DL5-induced inhibitory signaling in primary NK cells. Blockade of KIR2DL5 with blocking mAbs of the present technology significantly enhanced NK-mediated antitumor immunity both in vitro and in vivo, demonstrating blockade of the KIR2DL5/PVR pathway as an immunotherapy for treating human cancers.

The identification of KIR2DL5 as an inhibitory receptor of PVR adds KIR2DL5 into a complex regulatory network composed of the other 2 inhibitory receptors, TIGIT and CD96, and 1 activating receptor, DNAM-1, for PVR. Unlike TIGIT and CD96, which share a common binding site with DNAM-1 on PVR (Yu 2009), KIR2DL5 bound to a non-identical site on PVR and did not compete with those 3 receptors for PVR binding, suggesting a distinct mechanism by which KIR2DL5 exerts an inhibitory effect through engagement with PVR. KIR2DL5 mediated PVR⁺ tumor immune resistance to NK cell killing. Furthermore, KIR2DL5-mediated inhibition on NK cytotoxicity was abolished upon depletion of PVR on tumor cells. These findings support PVR as a primary ligand for KIR2DL5 to induce NK cell suppression and tumor immune evasion.

Allelic polymorphism significantly influences cell surface expression, antibody recognition, and ligand avidity of KIRs (Carr 2005; Campbell 2011). Distinct from UP-R1, which required both DO and D2 domain for KIR2DL5 recognition, anti-KIR2DL5 mAb F8B30 of the present technology bound to KIR2DL5 through the DO domain, suggesting that they recognize different epitopes on KIR2DL5. Besides 2DL5A*001 and DO variants, F8B30 also detected surface-expressed 2DL5A*005, the second most common 2DL5A allele in the human population, while UP-R1 failed to do so. Furthermore, PVR displayed a different binding capacity to different KIR2DL5 alleles. In comparison with 2DL5A*001, 2DL5B*00602 was moderately bound by PVR, while surface-expressed 2D5A*005 and 2DL5B*003 were not bound by PVR.

Crosstalk between NK cells and dendritic cells (DCs) via cytokines or direct cell-contact stimuli results in activation and cytokine production by both cell types, contributing to the coordination of innate and adaptive immune responses (Cooper 2004; Walzer 2005). The present technology demonstrates that KIR2DL5 significantly decreases production of a broad spectrum of cytokines and chemokines by NK cells, such as IFN-γ, TNF-α, and GM-CSF, which might subsequently impair NK cell-induced DC maturation and activation. PVR is highly expressed not only by tumor cells but also by some immune cell subsets, including DCs. TIGIT induces PVR phosphorylation and signaling in DCs, resulting in increased IL-10 and decreased IL-12 production by DCs (Yu 2009). DC-released IL-12 induces IFN-γ production and potentiate the cytotoxicity of NK cells (Biron 1999).

ITIM and ITSM sequences found in many inhibitory receptors are critical in transducing negative signaling through recruiting phosphatases, such as SHP-1 or SHP-2, upon tyrosine phosphorylation (Daeron 2008; Long 2008). The present tyrosine mutation study showed that both ITIM and ITSM were essential for KIR2DL5-mediated NK cell inhibition. KIR2DL5 recruited both SHP-1 and SHP-2 in primary human NK cells. Notably, both phosphorylated ITIM and ITSM contributed to KIR2DL5 association with SHP-1. KIR2DL5 association with SHP-2 completely relied on phosphorylated ITIM, but not ITSM. ITIM/SHP-1/SHP-2 and ITSM/SHP-1 inhibited the Vav1/ERK1/2/p90RSK and downstream NF-1B signaling pathway. These findings revealed the molecular basis for KIR2DL5-mediated suppression on NK cells.

Preclinical studies have demonstrated that the TIGIT/PVR axis is an attractive cancer immunotherapy target owing to its roles in modulating CD8⁺ T cell and NK cell responses (Andrews 2019; Yu 2009; Stanietsky 2009). However, TIGIT blockade monotherapy shows minimal effects on controlling tumor growth. Whereas dual blockade of TIGIT and PD-1/PD-L1 shows promising results in some experimental tumor models (Hung 2018; Johnston 2014; Dixon 2018) and in multiple trials (Bendell 2020; Niu 2022; Cohen 2021; Wainberg 2021; Rodriguez-Abreu 2020), combination of the anti-TIGIT antibody tiragolumab and the PD-L1 inhibitor atezolizumab failed to improve progression-free survival in a phase III extensive-stage small cell lung cancer trial (ClinicalTrials.gov NCT04256421).

As disclosed herein, the noncompetitive binding of KIR2DL5 and TIGIT to PVR suggested that both receptors can function simultaneously and independently and that blockade of the TIGIT/PVR axis would still leave the KIR2DL5/PVR pathway intact. TIGIT blockade had a minimal effect on NK cell cytotoxicity, whereas KIR2DL5 blockade markedly restored the cytolytic activity of NK cells. Thus, the existence of KIR2DL5-mediated inhibition on NK cells in the TME represents a substantial obstacle to the success of the blockade of TIGIT. KIR2DL5⁺ immune cells infiltrated in various human cancers that highly expressed PVR. Blockade of KIR2DL5 effectively inhibited tumor growth and improved mouse survival across multiple humanized mouse models.

In summary, the findings disclosed herein unraveled the cellular and molecular mechanisms underlying the inhibitory function of the KIR2DL5/PVR pathway, supporting that blockade of the immunosuppressive KIR2DL5/PVR axis alone or in combination with other therapies is a new therapeutic strategy.

### REFERENCES

1. Sharma P, Allison J P. Immune checkpoint targeting in cancer therapy: toward combination strategies with curative potential. Cell. 2015; 161(2):205-214.
2. Topalian S L, et al. Immune checkpoint blockade: a common denominator approach to cancer therapy. Cancer Cell. 2015; 27(4):450-461.
3. Sharma P, et al. Primary, adaptive, and acquired resistance to cancer immunotherapy. Cell. 2017; 168(4):707-723.
4. Jenkins R W, et al. Mechanisms of resistance to immune checkpoint inhibitors. Br J Cancer. 2018; 118(1):9-16.
5. Vivier E, et al. Functions of natural killer cells. Nat Immunol. 2008; 9(5):503-510.
6. Carlsten M, Childs R W. Genetic manipulation of NK cells for cancer immunotherapy: techniques and clinical implications. Front Immunol. 2015; 6:266.
7. Romee R, et al. Cytokine-induced memory-like natural killer cells exhibit enhanced responses against myeloid leukemia. Sci Transl Med. 2016; 8(357):357ra123.
8. Liu E, et al. Use of CAR-transduced natural killer cells in CD19-positive lymphoid tumors. N Engl J Med. 2020; 382(6):545-553.
9. Raulet D H, et al. Regulation of the natural killer cell receptor repertoire. Annu Rev Immunol. 2001; 19:291-330.
10. Long E O, et al. Controlling natural killer cell responses: integration of signals for activation and inhibition. Annu Rev Immunol. 2013; 31:227-258.
11. Lanier L L. Up on the tightrope: natural killer cell activation and inhibition. Nat Immunol. 2008; 9(5):495-502.
12. Cozar B, et al. Tumor-infiltrating natural killer cells. Cancer Discov. 2021; 11(1):34-44.
13. Andrews L P, et al. Inhibitory receptors and ligands beyond PD-1, PD-L1 and CTLA-4: breakthroughs or backups. Nat Immunol. 2019; 20(11):1425-1434.
14. Myers J A, Miller J S. Exploring the NK cell platform for cancer immunotherapy. Nat Rev Clin Oncol. 2021; 18(2):85-100.
15. Wagtmann N, et al. Molecular clones of the p58 NK cell receptor reveal immunoglobulin-related molecules with diversity in both the extra- and intracellular domains. Immunity. 1995; 2(5):439-449.
16. Vilches C, Parham P. KIR: diverse, rapidly evolving receptors of innate and adaptive immunity. Annu Rev Immunol. 2002; 20:217-251.
17. Winter C C, et al. Direct binding and functional transfer of NK cell inhibitory receptors reveal novel patterns of HLA-C allotype recognition. J Immunol. 1998; 161(2):571-577.
18. Frazier W R, et al. Allelic variation in KIR2DL3 generates a KIR2DL2-like receptor with increased binding to its HLA-C ligand. J Immunol. 2013; 190(12):6198-6208.
19. Vilches C, et al. KIR2DL5, a novel killer-cell receptor with a DO-D2 configuration of Ig-like domains. J Immunol. 2000; 164(11):5797-5804.
20. Cisneros E, et al. Allelic polymorphism determines surface expression or intracellular retention of the human NK cell receptor KIR2DL5A (CD158f). Front Immunol. 2016; 7:698.
21. Gomez-Lozano N, et al. Some human KIR haplotypes contain two KIR2DL5 genes: KIR2DL5A and KIR2DL5B. Immunogenetics. 2002; 54(5):314-319.
22. Du Z, et al. KIR2DL5 alleles mark certain combination of activating KIR genes. Genes Immun. 2008; 9(5):470-480.
23. Verschueren E, et al. The immunoglobulin superfamily receptome defines cancer-relevant networks associated with clinical outcome. Cell. 2020; 182(2):329-344.
24. Husain B, et al. A platform for extracellular interactome discovery identifies novel functional binding partners for the immune receptors B7-H3/CD276 and PVR/CD155. Mol Cell Proteomics. 2019; 18(11):2310-2323.
25. Shilts J, et al. A physical wiring diagram for the human immune system. Nature. 2022; 608(7922):397-404.
26. Takai Y, et al. Nectins and nectin-like molecules: roles in contact inhibition of cell movement and proliferation. Nat Rev Mol Cell Biol. 2008; 9(8):603-615.
27. Kucan Brlic P, et al. Targeting PVR (CD155) and its receptors in anti-tumor therapy. Cell Mol Immunol. 2019; 16(1):40-52.
28. Triki H, et al. CD155 expression in human breast cancer: Clinical significance and relevance to natural killer cell infiltration. Life Sci. 2019; 231:116543.
29. Carlsten M, et al. DNAX accessory molecule-1 mediated recognition of freshly isolated ovarian carcinoma by resting natural killer cells. Cancer Res. 2007; 67(3):1317-1325.
30. Castriconi R, et al. Natural killer cell-mediated killing of freshly isolated neuroblastoma cells: critical role of DNAX accessory molecule-1-poliovirus receptor interaction. Cancer Res. 2004; 64(24):9180-9184.
31. Masson D, et al. Overexpression of the CD155 gene in human colorectal carcinoma. Gut. 2001; 49(2):236-240.
32. Bottino C, et al. Identification of PVR (CD155) and Nectin-2 (CD112) as cell surface ligands for the human DNAM-1 (CD226) activating molecule. J Exp Med. 2003; 198(4):557-567.
33. Yu X, et al. The surface protein TIGIT suppresses T cell activation by promoting the generation of mature immunoregulatory dendritic cells. Nat Immunol. 2009; 10(1):48-57.
34. Chan C J, et al. The receptors CD96 and CD226 oppose each other in the regulation of natural killer cell functions. Nat Immunol. 2014; 15(5):431-438.
35. Bendell J C, et al. Phase Ia/Ib dose-escalation study of the anti-TIGIT antibody tiragolumab as a single agent and in combination with atezolizumab in patients with advanced solid tumors. Cancer Res. 2020; 80(16):CT302.
36. Niu J, et al. First-in-human phase 1 study of the anti-TIGIT antibody vibostolimab as monotherapy or with pembrolizumab for advanced solid tumors, including non-small-cell lung cancer (NSCLC). Ann Oncol. 2022; 33(2):169-180.
37. Cohen E, et al. SKYSCRAPER-09: a phase II, randomised, double-blinded study of atezolizumab (Atezo) plus tiragolumab (Tira) and atezo plus placebo as first-line (1L) therapy for recurrent/metastatic (R/M) PD-L1+squamous cell carcinoma of the head and neck (SCCHN). Ann Oncol. 2021; 32:814-815.
38. Wainberg Z, et al. Phase Ib study of the anti-TIGIT antibody tiragolumab in combination with atezolizumab in patients with metastatic esophageal cancer. Ann Oncol. 2021; 32:227-228.
39. Rodriguez-Abreu D, et al. Primary analysis of a randomized, double-blind, phase II study of the anti-TIGIT antibody tiragolumab (tira) plus atezolizumab (atezo) versus placebo plus atezo as first-line (1L) treatment in patients with PD-L1-selected NSCLC (CITYSCAPE). J Clin Oncol. 2020; 38(15):9503.
40. Ge Z, et al. TIGIT, the next step towards successful combination immune checkpoint therapy in cancer. Front Immunol. 2021; 12:699895.
41. Estefania E, et al. Human KIR2DL5 is an inhibitory receptor expressed on the surface of NK and T lymphocyte subsets. J Immunol. 2007; 178(7):4402-4410.
42. Cisneros E, et al. KIR2DL5: an orphan inhibitory receptor displaying complex patterns of polymorphism and expression. Front Immunol. 2012; 3:289.
43. Robinson J, et al. IPD—the Immuno Polymorphism Database. Nucleic Acids Res. 2010; 38(D1):863-869.
44. Moretta L. Dissecting CD56dim human NK cells. Blood. 2010; 116(19):3689-3691.
45. Lopez-Verges S, et al. CD57 defines a functionally distinct population of mature NK cells in the human CD56dimCD16+ NK-cell subset. Blood. 2010; 116(19):3865-3874.
46. Wu Y, et al. Developmental and functional control of natural killer cells by cytokines. Front Immunol. 2017; 8:930.
47. Zang X. New immune checkpoint pathways: HHLA2 and its receptors including TMIGD2. Paper presented at: Cold Spring Harbor Asia Conference on Precision Cancer Biology: From Targeted to Immune Therapies; Sep. 18-22, 2022; Suzhou, China. http://cshl.csh-asia.org/2017meetings/CANCER.html. Accessed Sep. 26, 2022.
48. Wei Y, et al. KIR3DL3-HHLA2 is a human immunosuppressive pathway and a therapeutic target. Sci Immunol. 2021; 6(61):eabf9792.
49. Bhatt R S, et al. KIR3DL3 is an inhibitory receptor for HHLA2 that mediates an alternative immunoinhibitory pathway to PD1. Cancer Immunol Res. 2021; 9(2):156-169.
50. Orange J S. Formation and function of the lytic NK-cell immunological synapse. Nat Rev Immunol. 2008; 8(9):713-725.
51. Treanor B, et al. Microclusters of inhibitory killer immunoglobulin-like receptor signaling at natural killer cell immunological synapses. J Cell Biol. 2006; 174(1):153-161.
52. Daeron M, et al. Immunoreceptor tyrosine-based inhibition motifs: a quest in the past and future. Immunol Rev. 2008; 224:11-43.
53. Huyer G, et al. Mechanism of inhibition of protein-tyrosine phosphatases by vanadate and pervanadate. J Biol Chem. 1997; 272(2):843-851.
54. Yusa S, et al. KIR2DL5 can inhibit human NK cell activation via recruitment of Src homology region 2-containing protein tyrosine phosphatase-2 (SHP-2). J Immunol. 2004; 172(12):7385-7392.
55. Pende D, et al. Killer Ig-like receptors (KIRs): their role in NK cell modulation and developments leading to their clinical exploitation. Front Immunol. 2019; 10:1179.
56. Carr W H, et al. KIR3DL1 polymorphisms that affect NK cell inhibition by HLA-Bw4 ligand. J Immunol. 2005; 175(8):5222-5229.
57. Campbell K S, Purdy A K. Structure/function of human killer cell immunoglobulin-like receptors: lessons from polymorphisms, evolution, crystal structures and mutations. Immunology. 2011; 132(3):315-325.
58. Cooper M A, et al. NK cell and DC interactions. Trends Immunol. 2004; 25(1):47-52.
59. Walzer T, et al. Natural-killer cells and dendritic cells: “l'union fait la force”. Blood. 2005; 106(7):2252-2258.
60. Biron C A, et al. Natural killer cells in antiviral defense: function and regulation by innate cytokines. Annu Rev Immunol. 1999; 17:189-220.
61. Long E O. Negative signaling by inhibitory receptors: the NK cell paradigm. Immunol Rev. 2008; 224:70-84.
62. Stanietsky N, et al. The interaction of TIGIT with PVR and PVRL2 inhibits human NK cell cytotoxicity. Proc Natl Acad Sci USA. 2009; 106(42):17858-17863.
63. Hung A L, et al. TIGIT and PD-1 dual checkpoint blockade enhances antitumor immunity and survival in GBM. Oncoimmunology. 2018; 7(8):e1466769.
64. Johnston R J, et al. The immunoreceptor TIGIT regulates antitumor and antiviral CD8(+) T cell effector function. Cancer Cell. 2014; 26(6):923-937.
65. Dixon K O, et al. Functional anti-TIGIT antibodies regulate development of autoimmunity and antitumor immunity. J Immunol. 2018; 200(8):3000-3007.
66. Zhao R H, et al. HHLA2 is a member of the B7 family and inhibits human CD4 and CD8 T-cell function. Proc Natl Acad Sci USA. 2013; 110(24):9879-9884.
67. Doench J G, et al. Optimized sgRNA design to maximize activity and minimize off-target effects of CRISPR-Cas9. Nat Biotechnol. 2016; 34(2):184-191.
68. Wang F, et al. RNAscope: a novel in situ RNA analysis platform for formalin-fixed, paraffin-embedded tissues. J Mol Diagn. 2012; 14(1):22-2 9.
69. Beziat et al. Deciphering the killer-cell immunoglobulin-like receptor system at super-resolution for natural killer and T-cell biology. Immunology 150(3):248-264 (2016).
70. Wojtowicz et al. A human IgF cell-surface interactome reveals a complex network of protein-protein interactions. Cell 182(4):1027-1043 (2020).
71. Yu et al. The surface protein TIGIT suppresses T cell activation by promoting the generation of mature immunoregulatory dendritic cells. Nat Immunol 10(1):48-57 (2009).

1. A method of increasing immune cell function in a subject comprising administering to the subject one or more agents that decrease KIR2DL5 expression and/or activity.

2. A method of treating an infectious disease in a subject in need thereof comprising administering to the subject one or more agents that decrease KIR2DL5 expression and/or activity.

3. A method of treating cancer in a subject in need thereof comprising administering one or more agents that decrease KIR2DL5 expression and/or activity.

4. The method of claim 1, wherein the one or more agents prevent or reduce KIR2DL5 binding to PVR poliovirus receptor (PVR).

5. The method of claim 4, wherein the one or more agents binds KIR2DL5 at or near its binding site for PVR.

6. The method of claim 4, wherein the one or more agents bind PVR at or near its binding site for KIR2DL5.

7. The method of claim 6, wherein binding of the one or more agents to PVR does not block PVR binding to TIGIT, DNAM-1, and CD96.

8. The method of claim 1, wherein the one or more agents is selected from a peptide, polypeptide, or small molecule.

9. The method of claim 8, wherein the polypeptide is an antibody or a fusion protein comprising said antibody.

10. The method of claim 8, wherein the antibody is a monoclonal antibody.

11. The method of claim 9, wherein the antibody is an antagonist antibody.

12. The method of claim 9, wherein the antibody or fusion protein comprising said antibody comprises a high chain variable region (VH) comprising an amino acid sequence encoded by SEQ ID NO:2, SEQ ID NO:6, SEQ ID NO:10, SEQ ID NO:14, SEQ ID NO:18, SEQ ID NO:22, SEQ ID NO:26, or SEQ ID NO:30.

13. The method of claim 9, wherein the antibody or fusion protein comprising said antibody comprises a VH region comprising an amino acid sequence encoded by a nucleotide sequence that is at least 80% identical to SEQ ID NO:2, SEQ ID NO:6, SEQ ID NO:10, SEQ ID NO:14, SEQ ID NO:18, SEQ ID NO:22, SEQ ID NO:26, or SEQ ID NO:30.

14. The method of claim 9, wherein the antibody or fusion protein comprising said antibody comprises a VH region comprising an amino acid of SEQ ID NO:3, SEQ ID NO:7, SEQ ID NO:11, SEQ ID NO:15, SEQ ID NO:19, SEQ ID NO:23, SEQ ID NO:27, or SEQ ID NO:31.

15. The method of claim 9, wherein the antibody or fusion protein comprising said antibody comprises a VH region comprising an amino acid sequence that is at least 80% identical to SEQ ID NO:3, SEQ ID NO:7, SEQ ID NO:11, SEQ ID NO:15, SEQ ID NO:19, SEQ ID NO:23, SEQ ID NO:27, or SEQ ID NO:31.

16. The method of claim 9, wherein the antibody or fusion protein comprising said antibody comprises a light chain variable region (LH) comprising an amino acid sequence encoded by SEQ ID NO:4, SEQ ID NO:8, SEQ ID NO:12, SEQ ID NO:16, SEQ ID NO:20, SEQ ID NO:24, SEQ ID NO:28, or SEQ ID NO:32.

17. The method of claim 9, wherein the antibody or fusion protein comprising said antibody comprises a LH region comprising an amino acid sequence encoded by a nucleotide sequence that is at least 80% identical to SEQ ID NO:4, SEQ ID NO:8, SEQ ID NO:12, SEQ ID NO:16, SEQ ID NO:20, SEQ ID NO:24, SEQ ID NO:28, or SEQ ID NO:32.

18. The method of claim 9, wherein the antibody or fusion protein comprising said antibody comprises a LH region comprising an amino acid of SEQ ID NO:5, SEQ ID NO:9, SEQ ID NO:13, SEQ ID NO:17, SEQ ID NO:21, SEQ ID NO:25, SEQ ID NO:29, or SEQ ID NO:33.

19. The method of claim 9, wherein the antibody or fusion protein comprising said antibody comprises a LH region comprising an amino acid sequence that is at least 80% identical to SEQ ID NO:5, SEQ ID NO:9, SEQ ID NO:13, SEQ ID NO:17, SEQ ID NO:21, SEQ ID NO:25, SEQ ID NO:29, or SEQ ID NO:33.

20. The method of claim 9, wherein the antibody is a chimeric antibody, a human antibody, or a humanized antibody.

21. The method of claim 2, wherein the infectious disease is caused by a pathogen.

22. The method of claim 21, wherein the pathogen is selected from a virus, bacterium, prion, fungus, parasite, or combination thereof.

23. The method of claim 22, wherein the virus is selected the group consisting of human immunodeficiency viruses, influenza viruses, papillomaviruses, coronaviruses, hepatitis viruses, and herpesviruses.

24. The method of claim 22, wherein the bacterium is Mycobacterium tuberculosis.

25. The method of claim 22, wherein the fungus is Pneumocystis jirovecii (PJP).

26. The method of claim 3, wherein the cancer is selected from the group consisting of chronic lymphocytic leukemia (CLL), acute leukemia, acute lymphoid leukemia (ALL), B-cell acute lymphoid leukemia (B-ALL), T-cell lymphoma, B-cell lymphoma, T-cell acute lymphoid leukemia (T-ALL), chronic myelogenous leukemia (CML), B-cell prolymphocytic leukemia, T-cell lymphoma, Hodgkin's disease, B-cell non-Hodgkin's lymphoma, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, mucosa-associated lymphoid tissue (MALT) lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenström macroglobulinemia, or preleukemia.

27. The method of claim 3, wherein the cancer is selected from the group consisting of colon cancer, rectal cancer, renal-cell carcinoma, liver cancer, lung cancer, kidney cancer, gastric cancer, gallbladder cancer, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers, combinations of the cancers, and metastatic lesions of the cancers.

28. The method of claim 3, wherein the cancer is a human hematologic malignancy.

29. The method of claim 28, wherein the human hematologic malignancy is selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt's lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

30. The method of claim 3, wherein the cancer is selected from the group consisting of bladder cancer, kidney cancer, breast cancer, lung cancer, liver cancer, brain cancer, prostate cancer, colon cancer, esophageal cancer, pancreatic cancer, uterine cancer, and stomach cancer.

31. The method of claim 3, wherein the cancer is a metastatic cancer.

32. The method of claim 3, further comprising administering the subject to one or more additional cancer therapies selected from chemotherapy, radiation therapy, immunotherapy, surgery, and a combination thereof.

33. A method of decreasing immune cell function in a subject comprising administering to the subject one or more agents that increase KIR2DL5 expression and/or activity.

34. A method of treating an autoimmune disease in a subject comprising administering to the subject one or more agents that increase KIR2DL5 expression and/or activity to the subject.

35. A method of decreasing transplant rejection in a subject comprising administering to the subject one or more agents that increase KIR2DL5 expression and/or activity to the subject.

36. The method of claim 33, wherein the one or more agents is selected from the group consisting of a peptide, polypeptide, and a small molecule.

37. The method of claim 36, wherein the polypeptide is a fusion protein or an antibody.

38. The method of claim 37, wherein the antibody is a monoclonal antibody.

39. The method of claim 37, wherein the antibody is an agonist antibody.

40. The method of claim 39, wherein the antibody increases activity of KIR2DL5.

41. The method of claim 37, wherein the antibody is a chimeric antibody, a human antibody, or a humanized antibody.

42. The method of claim 34, wherein the autoimmune disease is selected from the group consisting of acute disseminated encephalomyelitis (ADEM), alopecia areata, antiphospholipid syndrome, autoimmune cardiomyopathy, autoimmune hemolytic anemia, autoimmune hepatitis, autoimmune inner ear disease, autoimmune lipoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Behçet's disease, celiac disease, Chagas disease, cold agglutinin disease, Crohn's disease, dermatomyositis, diabetes mellitus type 1, eosinophilic fasciitis, gastrointestinal pemphigoid, Goodpasture's syndrome, Grave's syndrome, Guillain-Barré syndrome, Hashimoto's encephalopathy, Hashimoto's thyroiditis, lupus erythematosus, Miller-Fisher syndrome, mixed connective tissue disease, myasthenia gravis, pemphigus vulgaris, pernicious anemia, polymyositis, psoriasis, psoriatic arthritis, relapsing polychondritis, rheumatoid arthritis, rheumatic fever, Sjögren's syndrome, temporal arteritis, transverse myelitis, ulcerative colitis, undifferentiated connective tissue disease, vasculitis, Wegener's granulomatosis, and adult rheumatoid arthritis.

43. The method of claim 35, wherein the transplant is a stem cell transplant, bone marrow transplant, or combination thereof.

44. The method of claim 35, wherein the transplant is selected from the group consisting of a kidney transplant, a lung transplant, a heart transplant, a pancreas transplant, a cornea transplant, or a liver transplant.
